(19) United States
(12) Patent Application Publication
(10) Pub. No.: US 2016/0324897 A1
(43) Pub. Date: Nov. 10, 2016

(54) PLATELET DECOYS AND USE THEREOF

(71) Applicant: PRESIDENT AND FELLOWS OF HARVARD COLLEGE, Cambridge, MA (US)

(72) Inventors: Donald E. INGBER, Boston, MA (US); Anne-Laure PAPA, Boston, MA (US)

(73) Assignee: PRESIDENT AND FELLOWS OF HARVARD COLLEGE, Cambridge, MA (US)

(21) Appl. No.: 15/111,999

(22) PCT Filed: Jan. 16, 2015
(86) PCT No.: PCT/US15/11805

§ 371 Date: Jul. 15, 2016

Related U.S. Application Data
(60) Provisional application No. 61/928,458, filed on Jan. 17, 2014.
(60) Provisional application No. 61/938,329, filed on Feb. 11, 2014.

(51) Int. Cl. A61K 35/19; C12N 5/078

(52) U.S. Cl. CPC A61K 35/19; C12N 5/0644

(57) ABSTRACT

The invention provides platelet decoys and mimics that can bind to platelet receptor substrate but do not undergo platelet activation. The invention also provides methods of using the platelet decoys for treating, preventing or inhibiting a disease or disorder in subject when platelet activation, aggregation and/or adhesion contributes to the pathology or symptomology of the disease.

### RELATED APPLICATIONS

This application claims benefit under 35 U.S.C. §119(e) of the U.S. Provisional Application No. 61/928,458, filed Jan. 17, 2014, and No. 61/938,329, filed Feb. 11, 2014, the content of both of which is incorporated herein by reference in their entirety.

### TECHNICAL FIELD

The present disclosure relates generally to platelet decoys and mimics and methods of making and using same.

### BACKGROUND

Increased number of platelets is seen in cancer, myeloproliferative diseases and in chronic inflammatory conditions (Schafer A I. Thrombocytosis. N Engl J Med. 2004; 350(12):1211-9. Epub 2004 Mar. 19. doi: 10.1056/NEJMra035363. PubMed PMID: 15028825). This culminates with a strong possibility for these patients to develop thrombosis. An increased activation of platelets plays an important role in the pathogenesis of several diseases; this includes disseminated intravascular coagulation (DIC) (Levi M, van der Poll T. Disseminated intravascular coagulation: a review for the internist. Intern Emerg Med. 2013; 8(1):23-32. Epub 2012 Sep. 28. doi: 10.1007/s11739-012-0859-9. PubMed PMID: 23015284), sepsis (Li Z, Yang F, Dunn S, Gross A K, Smyth S S. Platelets as immune mediators: their role in host defense responses and sepsis. Thromb Res. 2011; 127(3):184-8. Epub 2010 Nov. 16. doi: 10.1016/j.thromres.2010.10.010. PubMed PMID: 21075430; PubMed Central PMCID: PMC3042496) and heparin-induced thrombocytopenia (HIT) (Warkentin T E. Heparin-induced thrombocytopenia: pathogenesis and management. Br J Haematol. 2003; 121(4):535-55. Epub 2003 May 20. PubMed PMID: 12752095). DIC is a pathology leading to generalized activation of platelets and the coagulation cascade. This results in the formation of microvascular clots and a resultant excessive consumption of platelets and coagulation proteins. It is often a complication of preexisting indications such as sepsis (Semeraro N, Ammollo C T, Semeraro F, Colucci M. Sepsis, thrombosis and organ dysfunction. Thromb Res. 2012; 129(3):290-5. Epub 2011 Nov. 9. doi: 10.1016/j.thromres.0.2011.10.013. PubMed PMID: 22061311), pulmonary embolism (Levi M. Disseminated intravascular coagulation or extended intravascular coagulation in massive pulmonary embolism. J Thromb Haemost. 2010; 8(7):1475-6. Epub 2010 Apr. 24. doi: 10.1111/j.1538-7836.2010.03891.x. PubMed PMID: 20412432; PubMed Central PMCID: PMC2905612), cancer (Hyman D M, Soff G A, Kampel L J. Disseminated intravascular coagulation with excessive fibrinolysis in prostate cancer: a case series and review of the literature. Oncology. 2011; 81(2):119-25. Epub 2011 Oct. 12. doi: 10.1159/000331705. PubMed PMID: 21986538) or amniotic fluid embolism (Moore J, Baldisseri M R. Amniotic fluid embolism. Crit Care Med. 2005; 33(10 Suppl):S279-85. Epub 2005 Oct. 11. PubMed PMID: 16215348). Platelet functions are involved in all these scenarios, often as facilitators of the disease progression or event. While DIC is a common pathway seen late in the pathogenesis of these diseases, activated platelets appear to play a very important driving role much earlier as well. They have been particularly shown as important players in sepsis and ARDS (Bachofen M, Weibel E R. Structural alterations of lung parenchyma in the adult respiratory distress syndrome. Clin Chest Med. 1982; 3(1):35-56. Epub 1982 Jan. 1. PubMed PMID: 7075161). New evidence also suggests that aspirin has a role in preventing recurrent DVT (Becattini C, Agnelli G, Schenone A, Eichinger S, Bucherini E, Silingardi M, et al. Aspirin for preventing the recurrence of venous thromboembolism. N Engl J Med. 2012; 366(21):1959-67. Epub 2012 May 25. doi: 10.1056/NEJMoa1114238. PubMed PMID: 22621626), this could be due to its inhibition of platelets.

Metastatic dissemination of the primary tumor is one of the main causes of death in cancer patients (H. B. P. Pearson, N., In Metastatic Cancer: Integrated Organ System and Biological Approach, Landes Bioscience ed.; Jandial, R. H., K., Ed. 2012). Since several decades, evidence has been accumulating that platelets play a crucial role in the metastatic cascade. Indeed, their support in regard to Circulating Tumor Cells (CTCs) is multifold: (i) shielding CTCs from shear stress in the bloodstream, (ii) protecting CTCs from the immune system, (iii) adhering to the endothelial wall so as to arrest CTCs within the vasculature, thus promoting new sites of metastasis and (iv) providing CTCs with pro-oncogenic and angiogenic factors that they release (N. M. Bambace, C. E. Holmes, The platelet contribution to cancer progression. J Thromb Haemost 9, 237-249 (2011); L. J. Gay, B. Felding-Habermann, Contribution of platelets to tumour metastasis. Nat Rev Cancer 11, 123-134 (2011); and G. L. Klement, T. T. Yip, F. Cassiola, L. Kikuchi, D. Cervi, V. Podust, J. E. Italiano, E. Wheatley, A. Abou-Slaybi, E. Bender, N. Almog, M. W. Kieran, J. Folkman, Platelets actively sequester angiogenesis regulators. Blood 113, 2835-2842 (2009)). Additionally, significantly high platelet counts (thrombocytosis) are found in 10 to 57% of cancer patients (E. Sierko, M. Z. Wojtukiewicz, Inhibition of platelet function: does it offer a chance of better cancer progression control? Semin Thromb Hemost 33, 712-721 (2007)), and are associated with poor prognosis (R. L. Stone, A. M. Nick, I. A. McNeish, F. Balkwill, H. D. Han, J. Bottsford-Miller, R. Rupairmoole, G. N. Armaiz-Pena, C. V. Pecot, J. Coward, M. T. Deavers, H. G. Vasquez, D. Urbauer, C. N. Landen, W. Hu, H. Gershenson, K. Matsuo, M. M. Shahzad, E. R. King, I. Tekedereli, B. Ozpolat, E. H. Ahn, V. K. Bond, R. Wang, A. F. Drew, F. Gushiken, D. Lamkin, K. Collins, K. DeGeest, S. K. Lutgendorf, W. Chiu, G. Lopez-Berestein, V. Afshar-Kharghan, A. K. Sood, Paraneoplastic thrombocytosis in ovarian cancer. N Engl J Med 366, 610-618 (2012) and P. Jurasz, D. Alonso-Escolano, M. W. Radomski, Platelet-cancer interactions: mechanisms and pharmacology of tumour cell-induced platelet aggregation. Br J Pharmacol 143, 819-826 (2004)). It has also been shown that in animal models, an absence of platelets causes a massive decrease in metastasis (L. A. Coupland, B. H. Chong, C. R. Parish, Platelets and P-selectin control tumor cell metastasis in an organ-specific manner and independently of NK cells. Cancer Res 72, 4662-4671 (2012)). This led to pursuit of three general approaches for anti-metastatic treatment based on platelet targeting in the past: reduction of cancer-associated thrombocytosis (H. V. Naina, S. Harris, Paraneoplastic thrombocytosis in ovarian cancer. N Engl J Med 366, 1840; author reply 1840 (2012)), elimination of platelets from bloodstream (L. A. Coupland, B. H. Chong, C. R. Parish, Platelets and P-selectin control tumor cell metastasis in an organ-specific manner and independently of NK cells. Cancer Res 72, 4662-4671 (2012) and G. J. Gasic, T. B. Gasic, C. C. Stewart, Antimetastatic effects associated with platelet reduction. Proc Natl Acad Sci USA 61, 46-52 (1968)) and inhibition of platelet function (M. Hejna, M. Raderer, C. C. Zielinski, Inhibition of metastases by anticoagulants. J Natl Cancer Inst 91, 22-36 (1999) and M. S. Cho, J. Bottsford-Miller, H. G. Vasquez, R. Stone, B. Zand, M. H. Kroll, A. K. Sood, V. Afshar-Kharghan, Platelets increase the proliferation of ovarian cancer cells. Blood 120, 4869-4872 (2012)). Numerous studies have been conducted in vitro and in vivo; however, there has been virtually no clinical translation of the basic findings that emerged from these studies.

There is need in the art for compositions and methods for producing and using platelet decoys that retain the ability to bind with platelet receptor substrates but do not aggregate or activate blood coagulation pathway.

### SUMMARY

The inventors have discovered that platelet membrane components are involved in platelet activation, aggregation, and/or adhesion and can be removed from the platelet without substantially affecting its ability to bind with ligands, including ligands on the surfaces of other platelets or on tumor cells that promote their activation and aggregation. Thus, aspects of the various embodiments disclosed herein are based, in part, on inventors' discovery of modified platelet cells or ‘decoys’ which retain the ability to bind with one or more cell ligands but do not undergo substantial activation when subjected conditions or agents used for platelet activation. Accordingly, in one aspect, the disclosure provides a platelet cell (e.g., a modified platelet cell or platelet decoy), which is capable of binding to a cell ligand or a platelet receptor substrate but does not undergo substantial aggregation or activates substantial blood coagulation when exposed or contacted with a condition or agent that activates platelets. In general, the modified platelet cell disclosed herein is substantially free of one or more membrane lipids or molecular components present in an unmodified platelet cell.

In some embodiments, the modified platelet cell has a lower or decreased amount of glycoprotein (GP) IIb/IIIa relative to an unmodified platelet cell. In some embodiments, the modified platelet cell has a lower or decreased amount of glycoprotein GPIb/IX/V relative to an unmodified platelet cell.

In some embodiments, the modified platelet cell comprises glycoprotein (GP) IIb/IIIa. In some embodiments, the GPIIb/IIIa does not become activated when the modified platelet is exposed or contacted with a condition or agent that activates platelets. In some embodiments, the modified platelet cell is capable of binding with a tumor cell. In some embodiments, the modified platelet cell is capable of binding (via a substrate, e.g. von Willebrand Factor) with a platelet, a modified platelet cell, an endothelial cell, or extracellular matrix.

In some embodiments, the modified platelet cell further comprises an antiplatelet agent. In some embodiments, the modified platelet cell further comprises an imaging agent or a drug to concentrate platelet modifying drugs to sites of platelet activation.

The disclosure also provides a method of preparing the platelet decoys disclosed herein. Generally, the method comprises inactivating one or more platelet receptors by removing or modifying membranes and other cytosolic or membrane components from a platelet, e.g., unmodified platelet. For example, removing or extracting or modifying one or more soluble lipids or molecular components from insoluble cellular and extracellular scaffolds of a platelet, e.g., unmodified platelet. In some embodiments, the platelets can be treated with a detergent to prepare the platelet decoy. Other exemplary methods for preparing the platelet decoys include, but are not limited to, treating the platelets with high salt, ammonium hydroxide, or a fixative. Without wishing to be bound by a theory, treatment conditions and time can be varied to obtain the desired degree of reduction in platelet function.

It is to be noted that the method of preparing the platelet decoys comprises extracting/removing/modifying membranes from the platelets. This is different from the art known ‘extraction’ or ‘washing’ methods for platelets. The art known ‘extraction’ or ‘washing’ methods for platelets inherently entail maintaining functional membranes. Thus, the art known methods will not provide a modified platelet cell which is capable of binding to a cell ligand or a platelet receptor substrate but does not undergo substantial aggregation or activates substantial blood coagulation when exposed or contacted with a condition or agent that activates platelets.

In some embodiments, the method for preparing the platelet decoys comprises increasing or enhancing expression of anti-apoptotic proteins in the platelets before the extraction/removing step.

In some embodiments, the method for preparing the platelet decoys comprises inhibiting, decreasing or repressing expression of molecules which play a part in identifying platelets for clearance before the extraction/removing step.

In another aspect, the disclosure provides a method of comprising administering to a subject a composition comprising a modified platelet cell disclosed herein. The platelet decoys can be administered to a subject in need of treating, preventing or inhibiting a disease or disorder when platelet activation, aggregation and/or adhesion contributes to the pathology or symptomology of the disease. In some embodiments, the platelet decoys can be administered to a subject in need of: (i) treating, preventing or inhibiting tumor cell metastasis or tumor cell interactions with platelets in the circulation; (ii) enhancing, increasing, or stimulating fibrinolysis; (iii) treating, preventing or suppressing excessive blood coagulation or clotting disorders; or (iv) enhancing, increasing, or stimulating clot formation at a platelet binding site, e.g., by delivering one or more agents that enhance, increase or stimulate clot formation; (v) imaging platelet adhesion, aggregation or blood clot formation; (vi) treating inflammation or inflammation associated disorders, as well as infections. Without wishing to be bound by a theory, the platelets and methods disclosed herein can prevent tumor cell aggregation that leads to tumor aggregate implantation and survival in small vessels

### BRIEF DESCRIPTION OF THE DRAWINGS

FIGS. 1A-1H show the effect of 50 μM TRAP (thrombin receptor activating peptide) (FIGS. 1B and 1F), 50 μM ADP (adenosine diphosphate) (FIGS. 1C and 1G) and 5 μg/mL collagen (FIGS. 1D and 1H) on platelet GP IIb/IIIa activation (also called CD41/CD61) in platelets (FIGS. 1B-1D) and platelet decoys (FIGS. 1F-1H). Platelets and platelet decoys were incubated with the different agonists before immunostaining with APC Mouse Anti-Human CD41a and PAC-1 FITC. PAC-1 selectively binds an epitope of activated GP IIb/IIIa complex. Flow cytometry dot plots show that platelets are significantly activated when they are incubated with agonists (FIGS. 1A-1D) while platelet decoys are not (FIGS. 1E-1H).

FIGS. 2A-2C show platelets and decoys activation assay based on PAC-1-FITC detection (FIG. 2A), CD41a (also called GPIIb) detection and evaluation of its agonist-induced surface recruitment (FIG. 2B) and CD42b (also called GPIb) surface detection (FIG. 2C) by flow cytometry.

FIGS. 3A-3L shows interaction of platelets and Decoys with breast cancer cell lines. Prior to flow cytometric analysis, platelets or Decoys were stained with a CD9-PE antibody (detection in y-axis) while cancer cells were stained with Hoechst (detection in x-axis) before being incubated together in plasma for 1 hour at 37° C. under agitation. Flow cytometry plots show: platelets (FIG. 3A), Decoys (FIG. 3B), MDA-MB-231 (FIG. 3C), MCF-7 (FIG. 3D) (in FIGS. 3A-3D, left panel (x₁, where x=a, b, c, d) is unstained and right panel (x₂, where x=a, b, c, d) is stained. The majority of cancer cells stained double positive for all conditions (FIGS. 3E-3H) as seen in the red highlighted upper right quadrant: platelets+MDA-MB-231 (FIG. 3E), Decoys+MDA-MB-231 (FIG. 3F), platelets+MCF-7 (FIG. 3G) and Decoys+MCF-7 (FIG. 3H). The interaction is impaired when platelets or decoys are pre-incubated with a GPIIb antibody (FIGS. 3I-3L).

FIGS. 4A-4E show aggregation response of platelets (P), Decoys (D) and mixtures of both at various P:D ratios, with (FIGS. 4A, 4B, 4D and 4E) or without (FIG. 4C) agonists: ADP (FIGS. 4A and 4D) and collagen (FIGS. 4B and 4E).

FIGS. 5A-5D show fluorescence imaging at 5 min (FIGS. 5A and 5C)) and intensity quantification (FIGS. 5B and 5D) of platelets and Decoys on incubation with collagen (FIGS. 5A and 5B) and fibrinogen (FIGS. 5C and 5D)-coated surfaces. Both platelets and Decoys were stained with CD9-FITC antibody and incubated 5, 10 and 15 min under agitation. Platelets were observed to aggregate onto these surfaces and this effect was amplified by the addition of 50 μM ADP. However, Decoys failed to attach to the collagen and fibrinogen-coated surfaces under the same conditions (with or without ADP).

FIG. 6 shows tPA immobilization at the surface of platelet Decoys evaluated by flow cytometry. Texas red-tPA was incubated with Decoys for 1 hour at room temperature and 4° C. Texas red was detected on the overall Decoy population even at 4° C. (blockade of energy dependent internalization pathways such as endocytosis), indicating that tPA has been immobilized onto their surface.

FIG. 7 shows Scanning Electron Micrographs (SEM) of resting platelet and platelet decoys.

FIG. 8 is a line graph showing adhesion of platelets, decoys and P:D and Abciximab-treated platelets on a collagen-coated microfluidic device (blood perfusion at 6.25 dyne/cm²).

### DETAILED DESCRIPTION

Aspects of the various embodiments disclosed herein are based on inventors' discovery that at least some platelets can be modified to inactivate platelet receptors by removing/extracting/modifying membranes and other cytosolic or membrane components without substantially affecting the platelet's ability to bind with ligands. Accordingly, the inventors have developed platelet decoys from platelets extracted from a subject that retain their ability to bind normal platelet (via ligand substrates), but fail to activate or trigger blood coagulation cascades.

In some embodiments, the platelet decoy retains at least one platelet receptor expressed in an unmodified platelet cell. In some embodiments, the platelet decoy comprises a functionally inactive form of the at least one platelet receptor expressed in the unmodified platelet. Exemplary receptors that are expressed in platelets include, without limitation, α5β1, α6β1, αvβ3, A2a adenosine receptor, Ax1/Tyro3/Mer, C1q receptor, CD100, CD110, CD111, CD112, CD114, CD114, CD147, CD148, CD151, CD154, CD165, CD17, CD29, CD31, CD36, CD40L, CD41 (GPIIb/IIIa complex), CD46, CD47, CD49, CD49b, CD51, CD55, CD58, CD60b, CD62P, CD63, CD72, CD82, CD84, CD9, CD93, CD98, CD99, CDw17, CDw32, CDw60, chemokine receptors, CLEC-2, c-mpl, C-type lectine receptors, dopamine receptor, Eph kinases, Ephr, ESAM, G6B, G6b-B, galectin receptors, Gas6 receptors, glutamate receptors, glycosaminoglycan-carrying receptors, GPI anchored proteins, GPIa-IIa, GPIb-IX-V complex, GPVI, ICAm-2, insulin receptor, JAMs, LAMP-1, LAMP-2, leptin receptor, liver x receptors, Lysophosphatidic acid receptors, P2 receptors, P2X₁, P2Y₁, P2Y₁₂, PAF receptors, protease activation receptors, PAR1, PAR4, PDGF receptor, PEAR1, PECAM-1, PGD₂, PGE₂, PGE₂ receptor (EP3), PGE₂ receptor (EP4), PGI₂ receptor (IP), PPARγ, P-selectin, PSGL-1, Semaphorin 3A receptors, serotonin receptor, Serotonin Reuptake receptor, Sphingosine-1-phosphate receptors, Tie-1 receptor, tight junction receptors, TLT-1, TNF receptor, toll-like receptors, TPα, TSSC6, TxA₂, V1a vasopressin receptor, VPAC1, α2B1, α5B1, α6B1, αvB3, and β2 adrenergic receptor.

In some embodiments, the platelet decoy disclosed herein comprises a functionally inactive form of glycoprotein (GP) complex IIb/IIIa thereof. Some exemplary platelet decoys disclosed herein comprise a functionally inactive GPIIb/IIIa complex that is capable of binding with an anti-CD41a. Glycoprotein IIb/IIIa (GPIIb/IIIa, also known as integrin αIIbβ3) is an integrin complex found on platelets. It is a receptor for fibrinogen and multivalent von Willebrand Factor (vWF) and aids in platelet activation and aggregation. The complex is formed via calcium-dependent association of GPIIb and GPIIIa, a required step in normal platelet aggregation and endothelial adherence. Platelet activation by ADP (blocked by clopidogrel) leads to a conformational change in platelet GPIIb/IIIa receptors that induces binding to fibrin. The GPIIb/IIIa receptor is a target of several drugs including abciximab, eptifibatide, and tirofiban.

In some embodiments, the platelet decoy disclosed herein comprises GPIIb/IIIa in an amount that is lower than relative to the amount of GPIIb/IIIa in an unmodified or naturally occurring platelet cell. For example, the amount of GPIIb/IIIa is at least 5% (e.g., 5%, 10%, 15%, 20%, 25%, 30%, 35%, 40%, 45%, 50%, 55%, 60%, 65%, 70%, 75%, 80%, 85%, 90%, 95% or more) lower than relative to the amount of GPIIb/IIIa in an unmodified or naturally occurring platelet cell. The unmodified or naturally occurring platelet cell can be the platelet cell from which the platelet decoy described herein is obtained.

In some embodiments, the GPIIb/IIIa is functionally inactive and does not activate when the platelet decoys is subjected to a condition or agent that activates platelets. For example, the platelet decoy fails to recruit additional GPIIb/IIIa receptors (from intracellular pools) and change the conformation of GPIIb/IIIa from its inactive to active form when subjected to platelet activation conditions. In some embodiments, the platelet decoys fail to express at least one epitope recognized by PAC-1, which is present in activated GPIIb/IIIa when subjected to platelet activation conditions.

In some embodiments, the platelet decoy disclosed herein comprises CD9 or a functionally inactive form thereof.

In some embodiments, the platelet decoy disclosed herein comprises GP Ib-IX-V or a functionally inactive form thereof. In some embodiments, the platelet decoy disclosed herein comprises GP Ib/IX/V in an amount that is lower than relative to the amount of GP Ib/IX/V in an unmodified or naturally occurring platelet cell. For example, the amount of GP Ib/IX/V is at least 5% (e.g., 5%, 10%, 15%, 20%, 25%, 30%, 35%, 40%, 45%, 50%, 55%, 60%, 65%, 70%, 75%, 80%, 85%, 90%, 95% or more) lower than relative to the amount of GP Ib/IX/V in an unmodified or naturally occurring platelet cell. The unmodified or naturally occurring platelet cell can be the platelet cell from which the platelet decoy described herein is obtained.

In some embodiments, the platelet decoy is substantially free of at least one component or at least one component functionality required or involved in platelet aggregation pathway and/or blood coagulation. In some embodiments, the at least one component or one component functionality required for platelet aggregation pathway and/or blood coagulation is a platelet surface receptor involved in platelet activation, aggregation and/or adhesion. As used herein, the term “free of” in relation to a platelet receptor or component means that the specified molecule is functionally absent. This can be due to the molecule being absent or is present but is functionally inactive. In some embodiments, a platelet receptor can be inactivated using an inhibitor.

Accordingly, in some embodiments of the various aspects disclosed herein, the platelet decoys are substantially free of one or more membrane lipids or molecular components present in an unmodified platelet cell. However, the platelets decoys disclosed herein retain one or more platelet components for binding with a cell ligand or a platelet receptor substrate. As used here, the term “cell ligand” or “platelet receptor substrate” or “platelet receptor ligand” refers to a ligand that binds to an epitope, antigen or receptor that is expressed on platelets. Exemplary platelet ligands include, but are not limited to, vWF, thrombin, FXI, FXII, P-selectin, HK, Mac-1, TSP-1, collagen, laminin, fibronectin, vitronectin, fibrinogen, fibrin, podoplanin, platelets CLEC-2, tumor cells, endothelial cells, and unmodified or modified platelet cells, B cell receptors, T cell receptors, lymphocyte receptors, neutrophil receptors, antigen-presenting cell receptors.

In some embodiments, the platelet decoy disclosed herein can bind extracellular matrix. In some embodiments, the platelet decoy disclosed herein can bind another platelet receptor substrate, e.g., a platelet decoy or an unmodified platelet cell. In some embodiments, the platelet decoy disclosed herein can bind an endothelial cell or white blood cells.

In some embodiments, the platelet decoy is substantially free of at least one platelet component selected from the group consisting of α5β1, α6β1, αvβ3, A2a adenosine receptor, Ax1/Tyro3/Mer, C1q receptor, CD100, CD110, CD111, CD112, CD114, CD114, CD147, CD148, CD151, CD154, CD165, CD17, CD29, CD31, CD36, CD40L, CD41 (GPIIb/IIIa complex), CD46, CD47, CD49, CD49b, CD51, CD55, CD58, CD60b, CD62P, CD63, CD72, CD82, CD84, CD9, CD93, CD98, CD99, CDw17, CDw32, CDw60, chemokine receptors, CLEC-2, c-mpl, C-type lectine receptors, dopamine receptor, Eph kinases, Ephr, ESAM, G6B, G6b-B, galectin receptors, Gas6 receptors, glutamate receptors, glycosaminoglycan-carrying receptors, GPI anchored proteins, GPIa-IIa, GPIb-IX-V complex, GPVI, ICAm-2, insulin receptor, JAMs, LAMP-1, LAMP-2, leptin receptor, liver x receptors, lysophosphatidic acid receptor, P2 receptors, P2X₁, P2Y₁, P2Y₁₂, PAF receptors, protease activation receptors, PAR1, PAR4, PDGF receptor, PDGF receptor, PEAR1, PECAM-1, PGD₂, PGE₂, PGE₂ receptor (EP3), PGE₂ receptor (EP4), PGI₂ receptor (IP), PPARγ, P-selectin, PSGL-1, Semaphorin 3A receptors, serotonin receptor, Serotonin Reuptake receptor, Sphingosine-1-phosphate receptors, Tie-1 receptor, tight junction receptors, TLT-1, TNF receptor, toll-like receptors, TPα, TSSC6, TxA₂, V1a vasopressin receptor, VPAC1, α2B1, α5B1, α6B1, αvB3, β2 adrenergic receptor, and any combinations thereof.

In some embodiments, the platelet decoy is substantially free of at least one platelet receptor or component that is required or needed for platelet recruitment, adhesion, activation or aggregation. Exemplary platelet receptors or components involved in recruitment, adhesion, activation or aggregation include, but are not limited to, GPIb-IX-V complex, GPVI, α2B1, α5B1, α6B1, αvB3, α2B1, α2B1, CD148, and CLEC-2.

In some embodiments, the platelet decoy is substantially free of at least one platelet receptor or component that is required or needed in amplification phase of the platelets. Exemplary platelet receptors or components involved in amplification phase of the platelets include, but are not limited to, P2Y₁, P2Y₁₂, PAR1, PAR4, TPα, TxA₂, PGE₂ receptor (EP3), PAF receptors, lysophosphatidic acid receptor, chemokine receptors, V1a vasopressin receptor, A2a adenosine receptor, β2 adrenergic receptor, serotonin receptor, dopamine receptor, P2X₁, c-mpl, leptin receptor, insulin receptor, and PDGF receptor.

In some embodiments, the platelet decoy is substantially free of one or more platelet receptors required or needed for the stabilization phase of platelet activation. Exemplary such receptors or components include, but are not limited to, Ephr, Ax1/Tyro3/Mer, P-selectin, TSSC6, CD151, CD36, TLT-1, and PEAR1.

In some embodiments, the platelet decoy is substantially free of at least one platelet receptor or component comprised in the negative regulation of platelet activation. Exemplary platelet receptors or components involved in negative regulation of platelet activation include but are not limited to VPAC1, PECAM-1, G6b-B, PGI₂ receptor (IP), PGD₂ receptor, PGE₂ receptor (EP4).

The balance between pro-apoptotic and anti-apoptotic molecules along with hepatic clearance are the major factors that govern the life span of platelets. These regulatory mechanisms can be delayed from identifying and clearing platelet decoys so as to enhance their life span thus, reducing the needed frequency of doses. Without wishing to be bound by a theory, this can be accomplished by increasing expression of anti-apoptotic proteins and/or repressing expression of molecules which play a part in identifying platelets for clearance.

Accordingly, in some embodiments, the platelet decoy comprises at least one anti-apoptotic protein or molecule in a higher or increased or enhanced amount or expression level relative to an unmodified or naturally occurring platelet. Exemplary anti-apoptotic proteins include, but are not limited to anti-apoptotic Bcl-2 proteins (e.g., Bcl-2, Bcl-xL, and ced-9), Bik, Hrk, Bad, Blk, stanniocalcin-1 (STC-1), stanniocalcin-2 (STC-2), FADD-like anti-apoptotic molecules (described in U.S. Pat. No. 6,207,801),

In some embodiments, the platelet decoy expresses an exogeneous anti-apoptotic protein gene. Methods for expressing exogenous genes in cells, such as in platelets, are well known in the art and one of ordinary skill in the art.

In some embodiments, amount or expression level of a molecule, which plays a part in identifying platelets for clearance. For example, Bak−/− platelets have been shown to have an extended life span (Josefsson et al. Megakaryocytes possess a functional intrinsic apoptosis pathway that must be restrained to survive and produce platelets. The Journal of experimental medicine. 2011; 208(10):2017-31) and consequently reduced levels in decoys could be very helpful in maintaining a significantly longer life span than platelets. In some embodiments, nucleic acid based technologies can be used for inhibiting or knocking down expression of nucleic acids encoding a molecule which plays a part in identifying platelets for clearance. Exemplary nucleic acid based technologies amenable for repressing or inhibiting expression of nucleic acids encoding such molecules include, but are not limited to, RNAi based nucleic acid (e.g., siRNAs, shRNAs, and miRNAs), anti-microRNA (e.g., antagomirs), antisense oligonucleotide, aptamers, ribozymes, triplex forming oligonucleotides. Exemplary molecules that may play a role in identifying platelets for clearance can include, but are not limited to, molecules that promote cell death (e.g., Bax, Bak, Bad, Bim, Bid and BCL-Xs).

In some embodiments, the platelet decoy can comprise an inhibitor of a pro-apoptotic proteins or molecules that promote cell death.

In some embodiments the platelet decoy further comprises an antithrombotic or thrombolytic agent or fibrinolytic agent. The antithrombotic or thrombolytic agent or fibrinolytic agent can be encapsulated in or coated on the platelet decoy. The antithrombotic or thrombolytic agent or fibrinolytic agent can be selected from the group consisting of anticoagulants, anticoagulant antagonists, antiplatelet agents, thrombolytic agents, thrombolytic agent antagonists, and any combinations thereof. Without limitations, the thrombogenic agent can be selected from the group consisting of thrombolytic agent antagonists, anticoagulant antagonists, pro-coagulant enzymes, pro-coagulant proteins, and any combinations thereof. Some exemplary thrombogenic agents include, but are not limited to, protamines, vitamin K1, aminocaproic acid (amicar), tranexamic acid (amstat), anagrelide, argatroban, cilstazol, daltroban, defibrotide, enoxaparin, fraxiparine, indobufen, lamoparan, ozagrel, picotamide, plafibride, tedelparin, ticlopidine, triflusal, collagen, collagen-coated particles, and any combinations thereof.

In some embodiments, the thrombogenic agent is thrombolytic agent. The thrombolytic agent can be encapsulated in or coated on the platelet decoy. As used herein, the term “thrombolytic agent” refers to any agent capable of inducing reperfusion by dissolving, dislodging or otherwise breaking up a clot, e.g., by either dissolving a fibrin-platelet clot, or inhibiting the formation of such a clot. Reperfusion occurs when the clot is dissolved and blood flow is restored. Exemplary thrombolytic agents include, but are not limited to, tissue-type plasminogen activator (t-PA), streptokinase (SK), prourokinase, urokinase (uPA), alteplase (also known as Activase®, Genentech, Inc.), reteplase (also known as r-PA or Retavase®, Centocor, Inc.), tenecteplase (also known as TNK™, Genentech, Inc.), Streptase® (AstraZeneca, LP), lanoteplase (Bristol-Myers Squibb Company), monteplase (Eisai Company, Ltd.), saruplase (also known as r-scu-PA and Rescupase™, Grunenthal GmbH, Corp.), staphylokinase, and anisoylated plasminogen-streptokinase activator complex (also known as APSAC, Anistreplase and Eminase®, SmithKline Beecham Corp.). Thrombolytic agents also include other genetically engineered plasminogen activators. The invention can additionally employ hybrids, physiologically active fragments or mutant forms of the above thrombolytic agents. The term “tissue-type plasminogen activator” as used herein is intended to include such hybrids, fragments and mutants, as well as both naturally derived and recombinantly derived tissue-type plasminogen activator. In some embodiments, the fibrinolytic agent is a tissue plasminogen activator.

Other exemplary thrombolytic agents include, but are not limited to, A-74187; ABC-48; adenosine for cardioprotection, King Pharma R&D; alfimeprase; alpha2-antiplasmin replacement therapy, Bayer; alteplase; amediplase; ANX-188; argatroban; arimoclomol; arundic acid (injectable formulation), Ono; asaruplase; ATH (thromboembolism/thrombosis), Inflazyme; atopaxar; BGC-728; bivalirudin; BLX-155; ciprostene; clazosentan; clomethiazole; clopidogrel; conestat alfa; CPC-211; desirudin; desmoteplase; DLBS-1033; DP-b99; DX-9065a; ebselen; echistatin, Merck & Co; edoxaban; efegatran; eptifibatide; erlizumab; EU-C-002; FK-419; fondaparinux sodium; H-290/51; hirudin-based thrombin inhibitors, BMS; HRC-102; ICI-192605; inogatran; lamifiban; lanoteplase; lumbrokinase; LY-210825; M5, Thrombolytic Science; melagatran; monteplase; MRX-820; nasaruplase; nicaraven; non-thrombolytic proteins, Genzyme; ocriplasmin (injected, stroke), Thrombogenics; ocriplasmin (ophthalmic), ThromboGenics/Alcon; ONO-2231; paclitaxel (lipid-based complex), MediGene; PB-007; PEGylated recombinant staphylokinase variant, ThromboGenics/Bharat Biotech; pexelizumab; Pro-UK; pro-urokinase, Erbamont; recombinant c1 esterase inhibitor (cardiovascular diseases), TSI; recombinant plasmin (vascular occlusion/ocular disease), Talecris Biotherapeutics/Bausch & Lomb; reteplase; saruplase; scuPA/suPAR (MI, stroke), Thrombotech; SM-20302; staplabin, Tokyo Noko; STC-387; SUPG-032; TA-993; TAFI inhibitors (thrombosis/myocardial infarction/stroke), Berlex; tenecteplase; TH-9229; THR-174; THR-18; tPA-HP; tridegin; troplasminogen alfa; urokinase; YM-254890; YM-337; YSPSL; and the like.

In some embodiments, the platelet decoy comprises an anti-coagulant agent. The anti-coagulant can be encapsulated in or coated on the platelet decoy. As used herein the term “anti-coagulant” is meant to refer to any agent capable of prolonging the prothrombin and partial thromboplastin time tests and reducing the levels of prothrombin and factors VII, IX and X. Anticoagulants typically include cormarin derivatives and heparin as well as aspirin, which can also be referred to as an antiplatelet agent. Some exemplary anti-coagulant agents include, but are not limited to, Warfin, Acenocoumarol, Phenindione, Dabigatran, Apixaban, Rivaroxaban, and the like.

In some embodiments, the platelet decoy further comprises an inhibitor of platelet activation, e.g., antiplatelet agent. The inhibitor can be encapsulated in or coated on the platelet decoy. As used herein, the term “antiplatelet agent” refers to any compound which inhibits activation, aggregation, and/or adhesion of platelets. Non-limiting examples of antiplatelet agents include adenosine diphosphate (ADP) antagonists or P2Y₁₂ antagonists, phosphodiesterase (PDE) inhibitors, adenosine reuptake inhibitors, Vitamin K antagonists, heparin, heparin analogs, direct thrombin inhibitors, glycoprotein IIb/IIIa inhibitors, anti-clotting enzymes, and nonsteroidal anti-inflammatory agents (NSAIDs).

In some embodiments, the antiplatelet agent is an ADP agonist or P2Y12 agonist. ADP antagonists or P2Y₁₂ antagonists block the ADP receptor on platelet cell membranes. This P2Y₁₂ receptor is important in platelet aggregation, the cross-linking of platelets by fibrin. The blockade of this receptor inhibits platelet aggregation by blocking activation of the glycoprotein IIb/IIIa pathway. Exemplary ADP antagonists or P2Y₁₂ antagonists include, but are not limited to, thienopyridine, sulfinpyrazone, ticlopidine, clopidogrel, prasugrel, R-99224 (an active metabolite of prasugrel, supplied by Sankyo), R-1381727, R-125690 (Lilly), C-1330-7, C-50547 (Millennium Pharmaceuticals), INS-48821, INS-48824, INS-446056, INS-46060, INS-49162, INS-49266, INS-50589 (Inspire Pharmaceuticals), Sch-572423 (Schering Plough), ticlopidine, sulfinpyrazone, ticlopidine, AZD6140, clopidogrel, prasugrel, clopidogrel bisulfate (PLA VIX™), clopidogrel hydrogen sulphate, clopidogrel hydrobromide, clopidogrel mesylate, cangrelor tetrasodium (AR-09931 MX), ARL67085, AR-C66096 AR-C 126532, and AZD-6140 (AstraZeneca), and any combinations thereof.

In some embodiments, the antiplatelet agent is a PDE inhibitor. A PDE inhibitor is a compound or composition that blocks one or more of the five subtypes of the enzyme phosphodiesterase (PDE), preventing the inactivation of the intracellular second messengers, cyclic adenosine monophosphate (cAMP) and cyclic guanosine monophosphate (cGMP), by the respective PDE subtype(s). Exemplary PDE inhibitors include, but are not limited to, cilostazol.

In some embodiments, the antiplatelet agent is an adenosine reuptake inhibitor. Adenosine reuptake inhibitors prevent the cellular reuptake of adenosine into platelets, red blood cells and endothelial cells, leading to increased extracellular concentrations of adenosine. These compounds inhibit platelet aggregation and cause vasodilation. Exemplary adenosine reuptake inhibitors include, but are no limited to, anagrelide, dipyridamole, pentoxifyllin, and theophylline.

In some embodiments, the antiplatelet agent is a vitamin K inhibitor. Vitamin K inhibitors are given to people to stop thrombosis (blood clotting inappropriately in the blood vessels). This is useful in primary and secondary prevention of deep vein thrombosis, pulmonary embolism, myocardial infarctions and strokes in those who are predisposed. Exemplary Vitamin K inhibitors include, but are not limited to, acenocoumarol, clorindione, dicumarol (Dicoumarol), diphenadione, ethyl biscoumacetate, phenprocoumon, phenindione, tioclomarol and warfarin.

In some embodiments, the antiplatelet agent is heparin or active fragments and fractions thereof from natural, synthetic, or biosynthetic sources. Heparin is a biological substance, usually made from pig intestines. It works by activating antithrombin III, which blocks thrombin from clotting blood. Examples of heparin and heparin substitutes include, but are not limited to, heparin calcium, such as calciparin; heparin low-molecular weight, such as enoxaparin and lovenox; heparin sodium, such as heparin, lipo-hepin, liquaemin sodium, and panheprin; heparin sodium dihydroergotamine mesylate; lithium heparin; ammonium heparin; antithrombin III; Bemiparin; Dalteparin; Danaparoid; Enoxaparin; Fondaparinux; Nadroparin; Parnaparin; Reviparin; Sulodexide; and Tinzaparin.

In some embodiments, the antiplatelet agent is a direct thrombin inhibitor. Direct thrombin inhibitors (DTIs) are a class of medication that act as anticoagulants (delaying blood clotting) by directly inhibiting the enzyme thrombin. Some exemplary DTIs include, but are not limited to, hirudin, bivalirudin (IV), lepirudin, desirudin, argatroban (IV), dabigatran, dabigatran etexilate (oral formulation), melagatran, and ximelagatran.

In some embodiments, the antiplatelet agent is a GPIIb/IIIa inhibitor. Glycoprotein IIb/IIIa inhibitors work by inhibiting the GPIIb/IIIa receptor on the surface of platelets, thus preventing platelet aggregation and thrombus formation. Exemplary glycoprotein IIb/IIIa inhibitors include, but are not limited to, abciximab, eptifibatide, tirofiban and prodrugs thereof.

In some embodiments, the antiplatelet agent is an anti-clotting enzyme. Some exemplary anti-clotting enzymes include, but are not limited to, Alteplase, Ancrod, Anistreplase, Brinase, Drotrecogin alfa, Fibrinolysin, Protein C, Reteplase, Saruplase, Streptokinase, Tenecteplase, and Urokinase.

As used herein, anticoagulants can also include factor Xa inhibitors, factor Ha inhibitors, and mixtures thereof. Various direct factor Xa inhibitors are known in the art including, those described in Hirsh and Weitz, Lancet, 93:203-241, (1999); Nagahara et al. Drugs of the Future, 20: 564-566, (1995); Pinto et al, 44: 566-578, (2001); Pruitt et al, Biorg. Med. Chem. Lett., 10: 685-689, (2000); Quan et al, J. Med. Chem. 42: 2752-2759, (1999); Sato et al, Eur. J. Pharmacol, 347: 231-236, (1998); Wong et al, J. Pharmacol. Exp. Therapy, 292:351-357, (2000). Exemplary factor Xa inhibitors include, but are not limited to, DX-9065a, RPR-120844, BX-807834 and SEL series Xa inhibitors. DX-9065a is a synthetic, non-peptide, propanoic acid derivative, 571 D selective factor Xa inhibitor. It directly inhibits factor Xa in a competitive manner with an inhibition constant in the nanomolar range. See for example, Herbert et al, J. Pharmacol. Exp. Ther. 276:1030-1038 (1996) and Nagahara et al, Eur. J. Med. Chem. 30(suppl):140s-143s (1995). As a non-peptide, synthetic factor Xa inhibitor, RPR-120844 (Rhone-Poulenc Rorer), is one of a series of novel inhibitors which incorporate 3-(S)-amino-2-pyrrolidinone as a central template. The SEL series of novel factor Xa inhibitors (SEL1915, SEL-2219, SEL-2489, SEL-2711: Selectide) are pentapeptides based on L-amino acids produced by combinatorial chemistry. They are highly selective for factor Xa and potency in the pM range.

Factor Ha inhibitors include DUP714, hirulog, hirudin, melgatran and combinations thereof. Melagatran, the active form of pro-drug ximelagatran as described in Hirsh and Weitz, Lancet, 93:203-241, (1999) and Fareed et al. Current Opinion in Cardiovascular, pulmonary and renal investigational drugs, 1:40-55, (1999).

In some embodiments, the anti-platelet agent can be selected from the group consisting of Ticlopidine, Clopidogrel, Prasugrel, Ticagrelor, Cangrelor, Elinogrel, Abciximab, Eptifibatide, Tirofiban, Dipyridamole, Cilostazol, Aspirin, Aggrenox, Ap4A derivatives, rCD39, Arthropod apyrase, rHuman Apyrase, terutroban, Ridogrel, Terbogrel, Picotamide, NCX-4016, Orofiban, Lotrafiban, Sibrafiban, Zemilofiban, RUC-1, Vorapaxar, Atopaxar, Anti-PAR4, Anti-PAR1, LIBS-Tap, 6B4-F_{ab}, H6B4-F_{ab}, Crotalin, Mamushigin, VCL, AjvW-2, Aurin Tricarboxylic acid, ARC1779, Kistomin, mF1232, cF1232, EMS-16, CTRP-1, Revacept, TGX221, Ketanserin, Sarpogrelate, APD791, DG-041, soluble Ax1 domains, Arp2/3 Antidody, rPSGL-Ig, PSI-697, PSI-421, CD40 antibody, steroidal glycosides, and any combinations thereof.

In some embodiments the platelet decoy further comprises a vasoconstrictor. The vasoconstrictor can be encapsulated in or coated on the platelet decoy. As used herein, the term “vasoconstrictor” refers to compounds or molecules that narrow blood vessels and thereby maintain or increase blood pressure, and/or decrease blood flow. There are many disorders that can benefit from treatment using a vasoconstrictor. For example, redness of the skin (e.g., erythema or cuperose), which typically involves dilated blood vessels, benefit from treatment with a vasoconstrictor, which shrinks the capillaries thereby decreasing the untoward redness. Other descriptive names of the vasoconstrictor group include vasoactive agonists, vasopressor agents and vasoconstrictor drugs. Certain vasoconstrictors act on specific receptors, such as vasopressin receptors or adrenoreceptors. Exemplary vasoconstrictors include, but are not limited to, alpha-adrenoreceptor agonists, chatecolamines, vasopressin, vasopressin receptor modualors, calcium channel agonists, and other endogenous or exogenous vasoconstrictors.

In some embodiments, the vasoconstrictor is selected from the group consisting of aluminum sulfate, amidephrine, amphetamines, angiotensin, antihistamines, argipressin, bismuth subgallate, cafaminol, caffeine, catecholamines, cyclopentamine, deoxyepinephrine, dopamine, ephedrine, epinephrine, felypressin, indanazoline, isoproterenol, lisergic acid diethylamine, lypressin (LVP), lysergic acid, mephedrone, methoxamine, methylphenidate, metizoline, metraminol, midodrine, naphazoline, nordefrin, norepinephrine, octodrine, ornipressin, oxymethazoline, phenylefhanolamine, phenylephrine, phenylisopropylamines, phenylpropanolamine, phenypressin, propylhexedrine, pseudoephedrine, psilocybin, tetrahydralazine, tetrahydrozoline, tetrahydrozoline hydrochloride, tetrahydrozoline hydrochloride with zinc sulfate, tramazoline, tuaminoheptane, tymazoline, vasopressin, vasotocin, xylometazoline, zinc oxide, and the like.

In some embodiments, the vasoactive agent is a substance derived or extracted from a herbal source, selected from the group including ephedra sinica (ma huang), polygonum bistorta (bistort root), hamamelis virginiana (witch hazel), hydrastis canadensis (goldenseal), lycopus virginicus (bugleweed), aspidosperma quebracho (quebracho bianco), cytisus scoparius (scotch broom), cypress and salts, isomers, analogs and derivatives thereof.

In some embodiments, the platelet decoy further comprises an agent known in the art for treatment of inflammation or inflammation associated disorders, or infections. Such agent can be encapsulated in or coated on the platelet decoy. Exemplary anti-inflammatory agents include, but are not limited to, non-steroidal anti-inflammatory drugs (NSAIDs—such as aspirin, ibuprofen, or naproxen), coricosteroids (such as presnisone), anti-malarial medication (such as hydrochloroquine), methotrexrate, sulfasalazine, leflunomide, anti-TNF medications, cyclophosphamise, mycophenolate, dexamethasone, rosiglitazone, prednisolone, corticosterone, budesonide, estrogen, estrodiol, sulfasalazine, fenfibrate, provastatin, simvastatin, proglitazone, acetylsalicylic acid, mycophenolic acid, mesalamine, hydroxyurea, and analogs, derivatives, prodrugs, and pharmaceutically acceptable salts thereof.

In some embodiments, the platelet decoy further comprises a vasodilator. The vasodilator can be encapsulated in or coated on the platelet decoy. A vasodilator can be selected from the group consisting of alpha-adrenoceptor antagonists (alpha-blockers), agiotensin converting enzyme (ACE) inhibitors, angiotensin receptor blockers (ARBs), beta2-adrenoceptor agonists (β2-agonists), calcium-channel blockers (CCBs), centrally acting sympatholytics, direct acting vasodilators, endothelin receptor antagonists, ganglionic blockers, nitrodilators, phosphodiesterase inhibitors, potassium-channel openers, renin inhibitors, and any combinations thereof. Exemplary vasodilator include, but are not limited to, prazosin, terazosin, doxazosin, trimazosin, phentolamine, phenoxybenzamine, benazepril, captopril, enalapril, fosinopril, lisinopril, moexipril, quinapril, ramipril, candesartan, eprosartan, irbesartan, losartan, olmesartan, telmisartan, valsartan, Epinephrine, Norepinephrine, Dopamine, Dobutamine, Isoproterenol, amlodipine, felodipine, isradipine, nicardipine, nifedipine, nimodipine, nitrendipine, clonidine, guanabenz, guanfacine, α-methyldopa, hydralazine, Bosentan, trimethaphan camsylate, isosorbide dinitrate, isosorbide mononitrate, nitroglycerin, erythrityl tetranitrate, pentaerythritol tetranitrate, sodium nitroprusside, milrinone, inamrinone (formerly amrinone), cilostazol, sildenafil, tadalafil, minoxidil, aliskiren, and analogs, derivatives, prodrugs, and pharmaceutically acceptable salts thereof.

In some embodiments, the platelet decoy further comprises an anti-neoplastic, anti-proliferative, and/or anti-mitotic agent. Such agents can be encapsulated in or coated on the platelet decoy. Exemplary anti-neoplastic/anti-proliferative/anti-mitotic agents include, but are not limited to, paclitaxel, 5-fluorouracil, doxorubicin, daunorubicin, cyclosporine, cisplatin, vinblastine, vincristine, epothilones, methotrexate, azathioprine, adriamycin and mutamycin; endostatin, angiostatin and thymidine kinase inhibitors, cladribine, taxol, trapidil, halofuginone, plasmin, and analogs, derivatives, prodrugs, and pharmaceutically acceptable salts thereof.

In some embodiment, the platelet decoy further comprises an anti-cancer agent. The anti-cancer agent can be encapsulated in or coated on the platelet decoy. Exemplary anti-cancer agents include, but are not limited to, A1-mafodotin; abagovomab; AC-01; ACH-1625; Ad/PSA; ADC-1009; adenovirus-mediated immunotherapy (melanoma), Zurich; ADVAX; ADXS-HER2; ADXS-HPV; Adxs-LmddA159; ADXS-PSA; AE-08; AE-298p; AE-37, Antigen Express; AEA-35p; AEH-10p; AE-M; AE-O; AEZS-120; AFTVac; AG-858; agatolimod; AGI-101H; AGS-003; AGS-005; AGS-006; AHICE; algenpantucel-L; AlloStim (infusion formulation, hematological neoplasms), Immunovative; ALT-212; ALVAC-CEA/B7.1; ALVAC-GM-CSF; ALVAC-KSA; amolimogene bepiplasmid; AMP-224; Anti-CEA antibody, Albert Einstein; ANZ-100; ANZ-521; AP-1903; APC-8020; APC-80TR; ApoVax104-HPV; ARGENT (prostate cancer therapy), ARIAD; ASP-0113; astuprotimut-r, GlaxoSmithKline; atazanavir; AVX-701; azacitidine; B7-1 gene therapy (in vivo/Ig G), Georgetown/Imperial College; B7-1 gene therapy, University of Wisconsin; balapiravir; BCL-002; BCL-003; BCL-004; BCL-005; belagenpumatucel-L; bendamustine; BHT-3009; BiovaxID; BIWB-1; BIWB-2; BN-500001; BN-600013; BP-16; BPX-101; BrevaRex; CAD-106; cadi-05; CAP1-6D; CBD1Qb; CBI-006; CBI-008; CC-394; CD40 ligand, Celldex; CDX-1127; CDX-1307; CDX-1401; CDX-2410; CDX-301; CeaVac; CEL-1000; Cervarix; CerVax-16; CG-201; choriogonadotropin alfa; ChronVac-C; CIGB-228; CIGB-247; CL-2000; CMVAC; CMX-001 (glioblastoma multiforme), California Pacific Medical Center; contusugene ladenovec; CPG23PANC; CRL-1005; CRM-197; CRS-207; CryoStim; CT-011; CT-201; CT-5; CTL (melanoma), Fred Hutchinson/Washington/Targeted Genetics; CTL-8004; CTP-37; CV-01; CV-07; CV-09; CV-301; CV-9103; CV-9201; CVac; CYT-003-QbG10; CYT-004-MelQbG10; CYT-005-allQbG10; CYT-006-AngQb; CYT-007-TNFQb; CYT-009-GhrQb; CYT-014-GIPQb; D-3263; daclatasvir; DCP-001; DCP-002; DEFB1 stimulators (peptide, prostate cancer), Phigenix; DISC-PRO; DPX-0907; DPX-Survivac; Drug Name; dSLIM (colon cancer), Mologen; DV-601; E7 toxoid; E-7300; EBV CTLs (EBV-associated lymphoma, nasopharyngeal carcinoma), Baylor College/Cell Medica; EC-708; EG-HPV; EG-Vac; elpamotide; EMD-249590; emepepimut-S; Engerix B; enkastim-ev; enkastim-iv; ENMD-0996; entinostat; enzalutamide; Epstein Barr-based gene therapy (intradermal, cancer), University of Birmingham; EradicAide; estradiol (transdermal, micro-encapsulated), Medicis/Novavax; ETBX-011; Eukaryotic Layered Vector System; F10 (neutralizing antibody, group 1 influenza A infection), Harvard Medical School/Dana-Farber Cancer Institute/XOMA/SRI International; F-50040; FAV-201; fibrovax, Cytokine; Folatelmmune; fosamprenavir; FP-03; FPI-01; Freevax; fresolimumab; fucosyl-GM1-KLH; fusogenic lipids, Liposome Company; Gardasil; gastrin 17C diphtheria toxoid conjugate (pancreatic cancer), Aster; gataparsen; GD2 ganglioside peptide mimics, Roswell Park Cancer Institute; Gemvac; gene therapy (Alzheimers), Somatix; gene therapy (anticancer), MediGene/Aventis; gene therapy (cancer), GenEra; gene therapy (cardiovascular), Somatix/Rockefeller; gene therapy (HPV), Chiron Viagene; gene therapy (HSV), Chiron Viagene; gene therapy (IL-2, cLipid), Valentis/Roche; gene therapy (prostate cancer), GenStar/Baxter; gene therapy (RTVP-1), Baylor College of Medicine; GI-10001; GI-4000; GI-5005; GI-6000; GI-6207; GI-6301; GI-7000; GL-0810; GL-0817; GL-ONC1; GM-CAIX; GM-CT-01; GMDP, Peptech; GMK; GnRH immunotherapeutic, ML/Protherics; golimumab; golotimod; Gonadimmune; gp53, ImClone; GPC-3298306; GPI-0100; GS-7977; GSK-2130579A; GSK-2241658A; GSK-2302024A; GSK-2302025A; GSK-2302032A; GSK-568893A; GV-1002; GV-1003; GVAX; GVX-3322; GX-160; GX-51; HE-2000; HepeX-B; Heplisav; HER-2 protein; HGP-30; HGTV-43; Homspera; HS-110; HS-210; HS-310; HS-410; HspE7; HSPPC-56; HSPPC-90; ICT-107; ICT-111; ICT-121; ICT-140; IDD-1; IDD-3; IDD-5; IDM-2101; IDN-6439; IEP-11; IGN-101; IGN-201; IGN-301; IGN-311; IGN-402; IGN-501; Ii-key/MHC class II epitope hybrid peptides (allergy), Antigen Express; IL-10; IL-12; IL-13; IMA-901; IMA-910; IMA-920; IMA-930; IMA-941; IMA-950; ImBryon; IMF-001; imiquimod; IMO-2055; IMP-321; IMP-361; IMT-1012; IMT-504; IMVAMUNE; IMX-MC1; IMX-MEL1; inactivated bacterial vectors; inCVAX; IndiCancerVac; indinavir; INGN-225; INNO-305; INO-5150; Insegia; interferon alfa-2b; interferon-gamma gene therapy (cancer), Chiron/Cell Genesys; interleukin-12 cancer vaccine, University of Wisconsin; interleukin-1beta, Celltech; interleukin-2 vaccine, ICR; inulin (gamma, ADVAX adjuvant), Vaxine; IPH-3102; IPH-3201; ipilimumab; ipilimumab+MDX-1379, Medarex/BMS; ipilimumab/IDD-1 combination vaccine (cancer), Medarex/IDM; IR-502; IRX-2; IRX-4; ISA-HPV-SLP; ISA-P53-01; ISCOMATRIX; KH-901; KLS-HPV; L19-IL-2 fusion protein, Philogen; L523S; lapuleucel-T; LG-768; LG-912; Lipomel; LMB-2; LMP-1/LMP-2 CTLs, Baylor College of Medicine/NCI; LN-020; LN-030; LN-040; LN-2200; lopinavir+ritonavir; Lovaxin M; Lovaxin NY; Lovaxin SCCE; Lovaxin T; LP-2307; LUD01-016; Lx-TB-PstSl; MAGE-3.A1 peptide (cancer), Ludwig Institute; MART-1 analogs, INSERM; Maxy-1200; MBT-2/VEGFR-2 MEDI-543; Melacine; Melan-A/IL-12, Genetics Institute; MEN-14358; MF-59; MGN-1601; MGV, Progenics; mifamurtide; MIS-416; mitumomab; mitumprotimut-t; MKC-1106-MT; MKC-1106-NS; MKC-1106-PP; ML-2400; MMU-18006; MTL-102; MTL-104; MUC1-Poly-ICLC; MUC2-KLH conjugate; Multiferon; Multikine; mutant ras; MVA-F6 vector (melanoma), Bavarian Nordic; MV-CEA; N-8295; necitumumab; nelfinavir; NeuroVax; NeuVax; Nfu-PA-D4-RNP; NGcGM3/VSSP (cancer), Recombio; NIC-002; Norelin; NSC-710305; NTX-010; NV-1020; OC-L vaccine (cancer), University of Pennsylvania; OCM-108; OCM-111; OCM-124; OCM-127; OCM-7342; OCV-101; OCV-105; OCV-501; ODC-0801; ODC-0901; oligodeoxynucleotides, Coley; Oligomodulators; oligonucleotide toll like receptor agonists; OM-174; OM-197-MP-AC; OM-294-DP; Oncophage; ONT-10; ONY-P; OPT-821; OPT-822; oregovomab; OTSGC-A24; OV-2500; P-17; P-501; PBT-2; PDS-0101; PDS-0102; PE64-delta-553pil; peginterferon alfa-2a; peginterferon alfa-2b; Pentarix; Pentrys; PEV-6; pexastimogene devacirepvec; PN-2300; POL-103A; Poly-ICLC; Polynoma-1; Polyshed-1; pradefovir; PRAME-SLP; Procervix; progenipoietin G; PRX-302; PS-2100; PSMA-ADC; PSMA-VRP; pSP-D-CD40L; pSP-D-GITRL; PT-107; PT-123; PT-128; PT-207; PVAC; PVX-410; QS-21; racotumomab; ranagengliotucel-T; resiquimod; rindopepimut; rintatolimod; RN-2500; RNF43-721; Roferon-A; RPK-739; S-288310; S-488210; S-488410; sargramostim; SART3 peptide; SCIB-1; SCIB-2; SD-101; SDZ-SCV-106; SFVeE6,7; SGD-2083; sialyl Lea-KLH conjugate; sipuleucel-T; SL-701; sLea-KLH; SLP; SP-1017; SRL-172; SSS-08; STxB-E7; SV-BR-1-GM; T1-IR; TA-CIN; TA-GW; TA-HPV; talimogene laherparepvec; TAP-1; TARP peptide; tasonermin; TBI-4000; technetium Tc 99m etarfolatide; TEIPP-01; tertomotide; TG-01, Targovax; TG-1024; TG-1031; TG-1042; TG-4010; TG-4040; TGF beta kinoid; TGF-alpha; TGFB2-antisense; Theradigm-CEA; Theradigm-Her-2; Theradigm-p53; Theradigm-prostate; Theramide; Theratope; thymalfasin; tipapkinogene sovacivec; TLR-7/TLR-8 agonists (cancer), Pfizer; TMX-202; TNF alpha kinoid; Tolamba; trametinib; TRC-105; tremelimumab; TriAb; TriGem; TRP-2 peptide-based therapeutic; TRX-385; TRX-518; TSD-0014; tucaresol; tucotuzumab celmoleukin; UltraCD40L; UltraGITRL; V-212; V-502; V-503; V-934/V-935; vadimezan; VB-1014; Vbx-011; Vbx-016; Vbx-021; Vbx-026; VEGF kinoid; Veldona; velimogene aliplasmid; VG-LC; VGX-3100; VGX-3200; VIR-501; vitalethine; vitespen; VLI-02A; VLI-02B; VLI-03B; VM-206; VPM-4001; WT-4869; XToll; and the like.

In some embodiments, the anti-cancer agent can be selected from the group consisting of paclitaxel (TAXOL®); docetaxel; germicitibine; Aldesleukin; Alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfanoral; calusterone; capecitabine; carboplatin; carmustine; carmustine with Polifeprosan Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); mechlorethamine (nitrogenmustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and any combinations thereof. In some embodiments, the anti-cancer agent is a paclitaxel-carbohydrate conjugate, e.g., a paclitaxel-glucose conjugate, as described in U.S. Pat. No. 6,218,367, content of which is herein incorporated by reference in its entirety.

In some embodiments, the platelet decoy can comprise an anti-angiogenic protein. For example, the anti-angiogenic protein can be encapsulated in or coated on the platelet decoy. Some exemplary anti-angiogenic proteins include, but are not limited to, Bevacizumab, Flt23k, CDP791, IMC-1121B, ranibizumab, VEGF-traps (e.g., aflibercept), Tie2-Fc, soluble fms-like tyrosine kinase-1(sFLT-1), angiostatin, endostatin, baculostatin, canstatin, maspin, PEX, tumstatin, anastellin, 16K PRL, thrombospondin-1(TSP-1), anginex, NOL7, 5179D prolactin, angioarrestin, platelet factor 4 (sPF4), double antiangiogenic protein (DAAP), laminin, protamine, a prolactin fragment, interferon alpha, anti-VEGF antibodies, anti-placental growth factor antibodies, anti-Flk-1 antibodies, anti-Fit-1 antibodies, plasminogen activator inhibitors, tissue metalloproteinase inhibitors, interleukin 12, interferon gamma-induced protein 10 (IP-10), gro-beta, proliferin-related protein, and angiopoietin 2.

The platelet decoys described herein can be used for imaging platelet adhesion, aggregation or blood clots, as well as detecting circulating tumor cells or any cells that platelet decoys can directly or indirectly bind. When used in imaging applications, the platelet decoys described herein typically comprise an imaging agent, which can be encapsulated in or coated on the platelet decoy. Accordingly, in some embodiments, the platelet decoy further comprises an imaging agent. As used herein, the term “imaging agent” refers to an element or functional group in a molecule that allows for the detection, imaging, and/or monitoring of the presence and/or progression of a condition(s), pathological disorder(s), and/or disease(s). The imaging agent can be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber.

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound. Suitable fluorescent reporters include xanthene dyes, such as fluorescein or rhodamine dyes, including, but not limited to, Alexa Fluor® dyes (InvitrogenCorp.; Carlsbad, Calif.), fluorescein, fluorescein isothiocyanate (FITC), Oregon Green™, rhodamine, Texas red, tetrarhodamine isothiocynate (TRITC), 5-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N,N′-tetramefhyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include 1-dimethylamino-naphthyl-5-sulfonate, 1-anilino-8-naphthalene sulfonate, 2-p-toluidinyl-6-naphthalene sulfonate, and 5-(2′-aminoethyl)aminonaphthalene-1-sulfonic acid (EDANS). Other fluorescent reporter dyes include coumarins, such as 3-phenyl-7-isocyanatocoumarin; acridines, such as 9-isothiocyanatoacridine and acridine orange; N-(p(2-benzoxazolyl)phenyl)maleimide; cyanines, such as Cy2, indodicarbocyanine 3 (Cy3), indodicarbocyanine 5 (Cy5), indodicarbocyanine 5.5 (Cy5.5), 3-(-carboxy-pentyl)-3′ethyl-5,5′-dimethyloxacarbocyanine (CyA); 1H,5H,11H, 15H-Xantheno[2,3,4-ij:5,6,74T]diquinolizin-18-ium, 9-[2(or 4)-[[[6-[2,5-dioxo-1-pyrrolidinyl)oxy]-6-oxohexyl] amino]sulfonyl]-4(or 2)-sulfophenyl]-2,3,6,7,12,13,16,17octahydro-inner salt (TR or Texas Red); BODIPY™ dyes; benzoxadiazoles; stilbenes; pyrenes; and the like. Many suitable forms of these fluorescent compounds are available and can be used.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al, Mol. Microbiol, 55:1767-1781 (2005), the GFP variant described in Crameri et al, Nat. Biotechnol., 14:315319 (1996), the cerulean fluorescent proteins described in Rizzo et al, Nat. Biotechnol, 22:445 (2004) and Tsien, Annu. Rev. Biochem., 67:509 (1998), and the yellow fluorescent protein described in Nagal et al, Nat. Biotechnol., 20:87-90 (2002). DsRed variants are described in, e.g., Shaner et al, Nat. Biotechnol., 22:1567-1572 (2004), and include mStrawberry, mCherry, mOrange, mBanana, mHoneydew, and mTangerine. Additional DsRed variants are described in, e.g., Wang et al, Proc. Natl. Acad. Sci. U.S.A., 101:16745-16749 (2004) and include mRaspberry and mPlum. Further examples of DsRed variants include mRFPmars described in Fischer et al, FEBS Lett., 577:227-232 (2004) and mRFPruby described in Fischer et al, FEBS Lett, 580:2495-2502 (2006).

Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane.

Suitable non-metallic isotopes include, but are not limited to, ¹¹C, ¹⁴C, ¹³N, ¹⁸F, ¹²³I, ¹²⁴I, ¹²⁵I, and ¹³¹I. Suitable radioisotopes include, but are not limited to, ⁹⁹mTc, ⁹⁵Tc, ¹¹¹In, ⁶²Cu, ⁶⁴Cu, Ga, ⁶⁸Ga, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and ¹⁵³Gd. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir.

A detectable response generally refers to a change in, or occurrence of, a signal that is detectable either by observation or instrumentally. In certain instances, the detectable response is fluorescence or a change in fluorescence, e.g., a change in fluorescence intensity, fluorescence excitation or emission wavelength distribution, fluorescence lifetime, and/or fluorescence polarization. One of skill in the art will appreciate that the degree and/or location of labeling in a subject or sample can be compared to a standard or control (e.g., healthy tissue or organ). In certain other instances, the detectable response is radioactivity (i.e., radiation), including alpha particles, beta particles, nucleons, electrons, positrons, neutrinos, and gamma rays emitted by a radioactive substance such as a radionuclide.

Specific devices or methods known in the art for the in vivo detection of fluorescence, e.g., from fluorophores or fluorescent proteins, include, but are not limited to, in vivo near-infrared fluorescence (see, e.g., Frangioni, Curr. Opin. Chem. Biol, 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramanujam et al, IEEE Transactions on Biomedical Engineering, 48:1034-1041 (2001), and the like. Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

Any device or method known in the art for detecting the radioactive emissions of radionuclides in a subject is suitable for use in the present invention. For example, methods such as Single Photon Emission Computerized Tomography (SPECT), which detects the radiation from a single photon gamma-emitting radionuclide using a rotating gamma camera, and radionuclide scintigraphy, which obtains an image or series of sequential images of the distribution of a radionuclide in tissues, organs, or body systems using a scintillation gamma camera, may be used for detecting the radiation emitted from a radiolabeled aggregate. Positron emission tomography (PET) is another suitable technique for detecting radiation in a subject.

The platelet decoys disclosed herein can also be used for delivering a therapeutic agent. Accordingly, in some embodiments, the platelet decoy can further comprise a therapeutic agent, which can be encapsulated in or coated on the platelet decoy. As used herein, the term “therapeutic agent” refers to a biological or chemical agent used for treatment, curing, mitigating, or preventing deleterious conditions in a subject. The term “therapeutic agent” also includes substances and agents for combating a disease, condition, or disorder of a subject, and includes drugs, diagnostics, and instrumentation. “Therapeutic agent” also includes anything used in medical diagnosis, or in restoring, correcting, or modifying physiological functions. The terms “therapeutic agent” and “pharmaceutically active agent” are used interchangeably herein.

The therapeutic agent is selected according to the treatment objective and biological action desired. General classes of therapeutic agents include anti-microbial agents such as adrenergic agents, antibiotic agents or antibacterial agents, antiviral agents, anthelmintic agents, anti-inflammatory agents, antineoplastic agents, antioxidant agents, biological reaction inhibitors, botulinum toxin agents, chemotherapy agents, diagnostic agents, gene therapy agents, hormonal agents, mucolytic agents, radioprotective agents, radioactive agents including brachytherapy materials, tissue growth inhibitors, tissue growth enhancers, vasoactive agents, thrombolytic agents (i.e., clot busting agents), and inducers of blood coagulation.

The therapeutic agent can be selected from any class suitable for the therapeutic objective. For example, if the objective is treating a disease, disorder or condition wherein platelet activation, aggregation or adhesion contributes to the pathology or symptomology of the disease, disorder or condition, the therapeutic agent can include antithrombotic or thrombolytic agent or fibrinolytic agents. By way of further example, if the desired treatment objective is treatment, prevention, or inhibition of tumor cell metastasis or tumor, the therapeutic agent can be anti-cancer agent or include radioactive material in the form of radioactive seeds providing radiation treatment directly into the tumor or close to it. Further, the therapeutic agent can be selected or arranged to provide therapeutic activity over a period of time.

Exemplary pharmaceutically active compound include, but are not limited to, those found in Harrison's Principles of Internal Medicine, 13^{th} Edition, Eds. T. R. Harrison McGraw-Hill N.Y., NY; Physicians Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete content of all of which are herein incorporated in its entirety.

In some embodiments of this and other aspects of the invention, the therapeutic agent is selected from the group consisting of aspirin, wafarin (coumadin), acenocoumarol, ancrod, anisindione, bromindione, clorindione, coumetarol, cyclocumarol, dextran, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol, dipyridamole (persantin), sulfinpyranone (anturane), ticlopidine (ticlid), tissue plasminogen activator (activase), plasmin, pro-urokinase, urokinase (abbokinase) streptokinase (streptase), and anistreplase/APSAC (eminase), and analogs, derivatives, prodrugs, and pharmaceutically acceptable salts thereof.

The therapeutic agent can be a radioactive material. Suitable radioactive materials include, for example, of ⁹⁰yttrium, ¹⁹²iridium, ¹⁹⁸gold, ¹²⁵iodine, ¹³⁷cesium, ⁶⁰cobalt, ⁵⁵cobalt, ⁵⁶cobalt, ⁵⁷cobalt, ⁵⁷magnesium, ⁵⁵iron, ³²phosphorous, ⁹⁰strontium, ⁸¹rubidium, ²⁰⁶bismuth, ⁶⁷gallium, ⁷⁷bromine, ¹²⁹cesium, ⁷³selenium, ⁷²selenium, ⁷²arsenic, ¹⁰³palladium, ¹²³lead, ¹¹¹Indium, ⁵²iron, ¹⁶⁷thulium, ⁵⁷nickel, ⁶²zinc, ⁶²copper, ²⁰¹thallium and ¹²³iodine. Without wishing to be bound by a theory, platelet decoys comprising a radioactive material can be used to treat diseased tissue such as tumors.

The various agents disclosed herein can be encapsulated in or coated on platelet decoys using methods known in the art and available to one of skill in the art. For example, the agent and the platelet decoy can be incubated together for a period of time to allow the agent either coat the platelet decoy or taken up by the platelet decoy. In some embodiments, the agent can be covalently linked to the surface of the platelet decoy.

In some embodiments, the platelet parents (i.e., from which the platelet decoys are obtained) can be modified to express a protein or molecule of interest. For example, the platelet parents can be transfected with a vector for expressing or silencing a protein or molecule of interest. In some embodiments, the protein or molecule of interest can be selected from the group consisting of antithrombotic agents, thrombolytic agents, fibrinolytic agents, anti-coagulant agents, an inhibitor of platelet activation, an anti-clotting enzyme, factor Xa inhibitors, vasoconstrictor, anti-inflammatory agents, vasodilator, anti-neoplastic agents, anti-proliferative agents, anti-miotic agents, anti-angiogenic protein, anti-cancer agents, imaging agents, therapeutic agents, and any combinations thereof. In some embodiments, the protein or molecule of interest can be a protein or molecule endogenous to the platelets. In some embodiments, the protein or molecule of interest can be a functionally inactive form of a protein or molecule present in unmodified platelets.

Generally, the method of preparing the platelet decoy comprises inactivating one or more platelet receptors by removing membranes and other cytosolic or membrane components from a platelet. Accordingly, milder versions of the various methods known in the art for extracting soluble components from biological cells can be used for preparing the platelet decoys disclosed herein. For example, the platelets extracted from whole blood can be modified with a light detergent such that modified platelets conserve some of their GP IIb/IIIa complex receptors, but that the activated conformation is not induced by stimulation with one or more of agonists. For example, in some embodiments, modified platelets conserve some of their GP IIb/IIIa complex receptors, but that the activated conformation is not induced by stimulation with one or more of ADP, TRAP, collagen, and the like.

In one embodiment, the method for preparing the modified platelets comprises extracting one or more soluble lipids or molecular components from insoluble cellular and extracellular scaffolds, such as the cytoskeleton and extracellular matrix of the platelet. This can be done by contacting or treating the platelets with a detergent or surfactant for a period of time to remove one or more soluble components from the platelets. The detergent or surfactant can be non-ionic or ionic (e.g., cationic). Exemplary ionic surfactants include, but are not limited to, octenidine dihydrochloride, alkyltrimethylammonium salts (e.g., cetyl trimethylammonium bromide (CTAB) a.k.a. hexadecyl trimethyl ammonium bromide, cetyl trimethylammonium chloride (CTAC)), cetylpyridinium chloride (CPC), benzalkonium chloride (BAC), benzethonium chloride (BZT), 5-Bromo-5-nitro-1,3-dioxane, dimethyldioctadecylammonium chloride, cetrimonium bromide, and dioctadecyldimethylammonium bromide (DODAB).

Exemplary zwitterionic surfactants include, but not limited to, sulfonates, such as CHAPS (3-[(3-Cholamidopropyl)dimethylammonio]-1-propanesulfonate); sultaines, such as cocamidopropyl hydroxysultaine; betaines, e.g., cocamidopropyl betaine; and phosphates, such as lecithin.

Exemplary non-ionic surfactants include fatty alcohols, cetyl alcohol, stearyl alcohol, and cetostearyl alcohol (consisting predominantly of cetyl and stearyl alcohols), and oleyl alcohol. Some specific example of non-ionic surfactants include polyoxyethylene glycol alkyl ethers (Brij) of formula: CH₃—(CH₂)₁₀₋₁₆—(O—C₂H₄)₁₋₂₅—OH (e.g., octaethylene glycol monododecyl ether, pentaethylene glycol monododecyl ether; polyoxypropylene glycol alkyl ethers of formula: CH₃—(CH₂)₁₀₋₁₆—(O—C₃H₆)₁₋₂₅—OH; glucoside alkyl ethers of formula: CH₃—(CH₂)₁₀₋₁₆—(O-Glucoside)₁₋₃-OH (such as decyl glucoside, lauryl glucoside, and octyl glucoside); polyoxyethylene glycol octylphenol ethers of formula: C₈H₁₇—(C₆H₄)—(O—C₂H₄)₁₋₂₅—OH (such as Triton X-100); polyoxyethylene glycol alkylphenol ethers of formula: C₉H₁₉—(C₆H₄)—(O—C₂H₄)₁₋₂₅—OH (e.g., Nonoxynol-9); glycerol alkyl esters such as glyceryl laurate; polyoxyethylene glycol sorbitan alkyl esters, such as polysorbate; sorbitan alkyl esters, such as Spans; cocamide MEA; cocamide DEA; dodecyldimethylamine oxide; block copolymers of polyethylene glycol and polypropylene glycol, e.g., Poloxamers; and polyethoxylated tallow amine (POEA).

In some embodiments, the detergent or surfactant can be selected from Triton (Triton-X100 and other Triton family members), Nonidet, octylphenol ethoxylates, nonyl phenoxypolyethoxylethanol (NP-40), octylphenoxypolyethoxyethanol (Nonidet P-40), and the like.

Without wishing to be bound by a theory, the time and concentration of detergent treatment can be modulated depending on the desired degree of reduction of platelet function as well. For example, treatment can be for a period of 60 minutes, 45 minutes, 30 minutes, 25 minutes, 20 minutes, 15 minutes, 10 minutes, 5 minutes, 4 minutes, 3 minutes, 2 minutes, 1 minute, 55 seconds, 50 second, 45 second, 40 second, 35 seconds, 30 seconds, 25 seconds, 20 seconds, 15 seconds, 10 seconds or less. Accordingly, in some embodiments, the detergent treatment can be for a period of few seconds to minutes. In some embodiments, the detergent treatment can be for a period of about 1 to about 60 seconds, from about 2 seconds to about 45 seconds, from about 3 seconds to about 30 seconds, or from about 5 seconds to about 15 seconds. The detergent treatment can be repeated one or more times (e.g., one, two, three, four, five, six, seven, eight, nine, ten, or more times), for example, if a more complete inactivation of the decoys is desired.

Alternative methods for obtaining Platelet Decoys can employ fixation of platelets using a short exposure to commercially available fixatives (such as aldehydes), or extraction using high salt, ammonium hydroxide or other buffers that have been shown to remove soluble lipids and molecular components from insoluble cellular and extracellular scaffolds, such as the cytoskeleton and extracellular matrix. Some methods for obtaining Platelet Decoys can employ a cocktail of both detergent and fixative.

After production, the platelet decoys can be stored in suitable buffer before use or platelet poor plasma (PPP). An exemplary buffer for platelet storage is the HEPES-Tyrode buffer or any variant of this saline buffer.

In some embodiments, the method of preparing the platelet decoy further comprises obtaining platelets from a subject. The subject can be a healthy subject or a subject in need of treatment for a disease or disorder where platelet activation, aggregation, and/or adhesion contributes to the pathology or symptomology of the disease or disorder. Methods of obtaining platelets from a subject are well known in the art and easily available to one of skill in the art.

The activation or lack thereof of the platelet decoys can be tested by (i) quantifying formation of aggregates containing platelets and fibrin; (ii) ADP release detection; and/or (iii) PAC-1 binding or P-selectin evaluation.

In some embodiments, the platelet decoy binds a cancer cell, e.g. a circulating tumor cell. As a result, these platelet decoys can dilute and compete with existing platelets in the bloodstream for binding with platelet receptor substrates and circulating tumor cells (CTCs). Binding with the CTCs can decrease the ability of CTCs to induce platelet activation, coagulation, and other platelet responses that are required to facilitate tumor cell extravasation from the vasculature.

Given the low incidence of CTCs in the circulation, their normal low likelihood of survival, and the fact that cancer patients already have abnormally high levels of hyper-activated platelets, platelet decoys disclosed herein can be administered to a subject and suppress tumor metastasis without producing systemic bleeding or other side effects. In addition, the platelet decoys can be useful for treatment of diseases or conditions that are associated with increased platelet aggregation or blood emboli formation, such as thrombotic thrombocytopenic purpura (TTP), stroke, disseminated intravascular coagulation (DIC) or other coagulopathies that either depend on platelet activation or lead to platelet consumption. Inflammatory diseases where platelets are activated and drive a systemic inflammatory response can also benefit from ‘platelet decoys’ due to the lack of secretion of platelet-derived mediators, such as sepsis (W. C. Aird, The hematologic system as a marker of organ dysfunction in sepsis. Mayo Clinic proceedings. Mayo Clinic 78, 869-881 (2003)) and acute respiratory distress syndrome (ARDS) (J. N. Katz, K. P. Kolappa, R. C. Becker, Beyond thrombosis: the versatile platelet in critical illness. Chest 139, 658-668 (2011)).

Accordingly, in one aspect, the disclosure provides a method comprising administering to a subject a platelet decoy disclosed herein. In some embodiments, the subject is need of treating, preventing or inhibiting a disease or disorder when platelet activation, aggregation, adhesion, and/or increased platelet number contributes to the pathology or symptomology of the disease. In some embodiment, the disease or disorder is a coagulopathy.

Without limitations, the platelets and methods disclosed herein can be used in treating, preventing or inhibiting coagulapathies and diseases that are caused by high platelet levels or hyper-active platelet function (thrombophilia). Examples include, but are not limited to, disseminated intravascular coagulation due to hyperactivation of platelets. This is commonly induced by sepsis and seen in cancer as well. Also patients with history of deep vein thrombosis (DVT) have this problem, and there are genetic syndromes as well. Additonal hypercoagulability diseases include, such as Antithrombin III deficiency, Protein C deficiency, Activated protein C resistance, Protein S deficiency, Factor V Leiden, Hyperprothrombinemia; and essential thrombocytosis. Other diseases include, polycythemia vera, myeloproliferative disorders, sickle cell disease, nephrotic syndrome, inflammatory bowel disease, pregnancy, and the like.

In some embodiments, the subject is in need of treating, preventing or inhibiting tumor cell metastasis or tumor formation. In some embodiments, the subject is in need of treating, preventing or inhibiting tumor cell metastasis or tumor cell formation and the method further comprises co-administering an anti-cancer therapy or treatment to the subject.

In some embodiments, the platelet disclosed herein can be used for imaging platelet adhesion, aggregation, blood clot formation in a subject or to detect any cells that platelet decoys can directly or indirectly bind.

In one embodiment, the compositions and methods disclosed herein are useful for a subject who has cancer regression. In another embodiment, the compositions and methods disclosed herein are useful for a subject who has a therapy resistant cancer, for example a chemotherapy resistant cancer. In some embodiments, the compositions and methods disclosed herein are useful for a subject who has cancer and has been exposed to adjuvant cancer therapies. In another embodiment, the compositions and methods disclosed herein are useful for a subject with a malignant cancer. In some embodiments, the compositions and methods disclosed herein are useful for a subject with a cancer. In some embodiments, the compositions and methods disclosed herein are also useful in the treatment of other disease or disorders associated with abnormal cellular proliferation. Thus, treatment can be directed to a subject who is affected but asymptomatic with cancer, for example, a disease of an organ or tissue in a subject characterized by poorly controlled or uncontrolled multiplication of normal or abnormal cells in that tissue and its effect on the body as a whole.

In some embodiments, the subject is in need of enhancing, increasing, or stimulating fibrinolysis. In some other embodiments, the subject is in need of enhancing, increasing, or stimulating blood coagulation or clot formation.

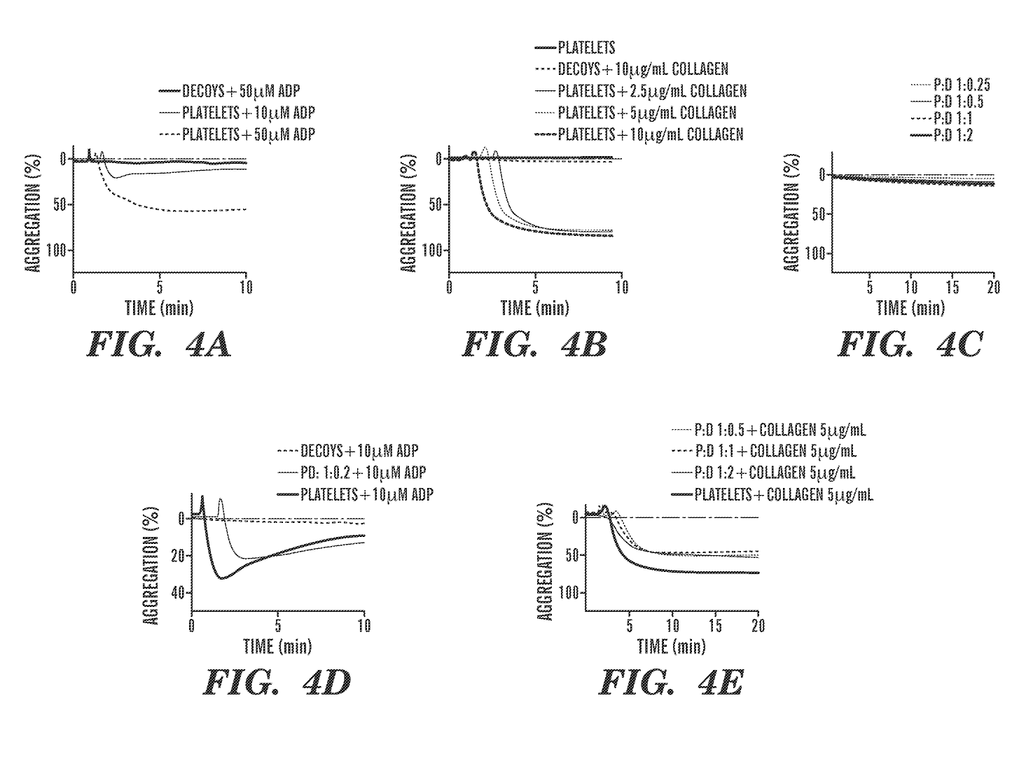

In some embodiments, the platelet decoys can be used as an adjunctive antiplatelet therapy in the setting of a Percutaneous Coronary Intervention (PCI). This can be useful for Acute Coronary Syndrome (ACS) when revascularization employs Percutaneous Coronary Intervention. As shown in the Examples section, platelet decoys do not self-aggregate under agonist stimuli (FIGS. 4A and 4B). Furthermore, they are able to mitigate platelet aggregation under agonist when decoys and platelets are co-incubated. This has been shown using both 5 μg/mL collagen (FIG. 4E) and 10 μM ADP (FIG. 4D) as agonists. Thus, the platelet decoys disclosed here can be used as anti-platelet agents during revascularization employing Percutaneous Coronary Intervention. The platelet decoys can be administered intravenously. Without wishing to be bound by a theory, the platelet decoys can provide a rapid onset of platelet inhibition after administration.

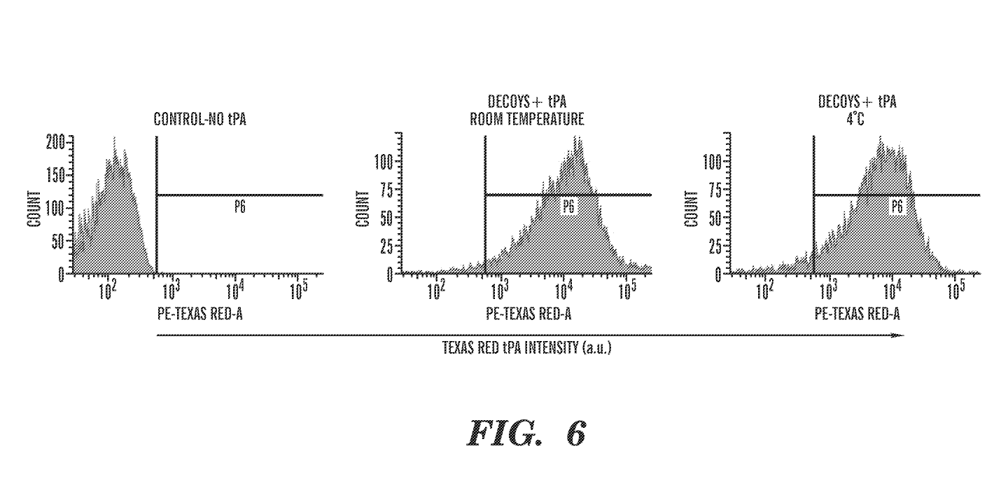

In some embodiments, the platelet decoys disclosed herein can be as an adjunct to fibrinolytic therapy for ACS. Standard regimens of emergency fibrinolytic therapy in myocardial infarction and stroke consist of a combination of fibrinolytic tPA (tissue Plasminogen Activator) and anti-platelet therapy. Literature has shown that platelets potentiate the activation of inactive circulatory plasminogen by tPA and it has been hypothesized that the potentiating effect on fibrinolysis is due to the co-localization of tPA and plasminogen on the platelets' surface (Platelets by A D Michelson, Elsevier, 3rd edition, 2013). Data reported in the Examples sections shows significant immobilization of Texas red-labeled tPA on Platelet Decoys (FIG. 6). Thus, tPA tagged platelet decoys can be used as a single injection fibrinolytic that can reduce hemorrhagic side effects by focusing tPA activity at the specific site of the clot.

In some embodiments, the method further comprises co-administering a therapy or agent known in the art for the disease or disorder for which the subject is in need of treating or preventing. As used herein, the term “co-administer” refers to administration of two or more therapies (e.g., platelet decoys and a therapy or pharmaceutically active agent) within a 24 hour period of each other, for example, as part of a clinical treatment regimen. In other embodiments, “co-administer” refers to administration within 12 hours, within 6 hours, within 5 hours, within 4 hours, within 3 hours, within 2 hours, within 1 hour, within 45, within 30 minutes, within 20, within 15 minutes, within 10 minutes, or within 5 minutes of each other. In other embodiments, “co-administer” refers to administration at the same time, either as part of a single formulation or as multiple formulations that are administered by the same or different routes. When the platelet decoys and the pharmaceutically active agent are administered in different pharmaceutical compositions or at different times, routes of administration can be same or different. In some embodiments, the method further comprises co-administering an antithrombotic or thrombolytic agent, a fibrinolytic agent, an anti-coagulant agent, antiplatelet agent, a vasodilator, an anti-neoplastic or anti-proliferative or anti-mitotic agent, an anti-inflammatory agent, an anti-cancer agent, or any combinations thereof to the subject.

In some embodiments, the method further comprises co-administering an anti-cancer therapy, agent or vaccine to the subject. As used herein, an anti-cancer treatment aims to reduce, prevent or eliminate cancer cells or the spread of cancer cells or the symptoms of cancer in the local, regional or systemic circulation. Anti-cancer treatment also means the direct treatment of tumors, for example by reducing or stabilizing their number or their size (curative effect), but also by preventing the in situ progression of tumor cells or their diffusion, or the establishment of tumors; this also includes the treatment of deleterious effects linked to the presence of such tumors, in particular the attenuation of symptoms observed in a patient or an improvement in quality of life. By “reduced” in the context of cancer is meant reduction of at least 10% in the growth rate of a tumor or the size of a tumor or cancer cell burden.

Cancer therapy can also include prophylaxis, including agents which slow or reduce the risk of cancer in a subject. In some embodiments, then anti-cancer treatment is an agent which suppresses the EGF-EGFR pathway, for example but not limited to inhibitors and agents of EGFR. Inhibitors of EGFR include, but are not limited to, tyrosine kinase inhibitors such as quinazolines, such as PID 153035, 4-(3-chloroanilino) quinazoline, or CP-358,774, pyridopyrimidines, pyrimidopyrimidines, pyrrolopyrimidines, such as CGP 59326, CGP 60261 and CGP 62706, and pyrazolopyrimidines, 4-(phenylamino)-7H-pyrrolo[2,3-d]pyrimidines (Traxleretal., (1996) J. Med Chem 39:2285-2292), curcumin (diferuloyl methane) (Laxmin arayana, et al., (1995), Carcinogen 16:1741-1745), 4,5-bis (4-fluoroanilino)phthalimide (Buchdunger et al. (1995) Clin. Cancer Res. 1:813-821; Dinney et al. (1997) Clin. Cancer Res. 3:161-168); tyrphostins containing nitrothiophene moieties (Brunton et al. (1996) Anti Cancer Drug Design 11:265-295); the protein kinase inhibitor ZD-1 839 (AstraZeneca); CP-358774 (Pfizer, Inc.); PD-01 83805 (Warner-Lambert), EKB-569 (Torrance et al., Nature Medicine, Vol. 6, No. 9, September 2000, p. 1024), HKI-272 and HKI-357 (Wyeth); or as described in International patent application WO05/018677 (Wyeth); WO99/09016 (American Cyanamid); WO98/43960 (American Cyanamid); WO 98/14451; WO 98/02434; WO97/38983 (Warener Lambert); WO99/06378 (Warner Lambert); WO99/06396 (Warner Lambert); WO96/30347 (Pfizer, Inc.); WO96/33978 (Zeneca); WO96/33977 (Zeneca); and WO96/33980 (Zeneca), WO 95/19970; U.S. Pat. App. Nos. 2005/0101618 assigned to Pfizer, 2005/0101617, 20050090500 assigned to OSI Pharmaceuticals, Inc.; all herein incorporated by reference. Further useful EGFR inhibitors are described in U.S. Pat. App. No. 20040127470, particularly in tables 10, 11, and 12, and are herein incorporated by reference.

In another embodiment, the anti-cancer therapy includes administering a cytostatic agent. A cytostatic agent is any agent capable of inhibiting or suppressing cellular growth and multiplication. Examples of cytostatic agents used in the treatment of cancer are paclitaxel, 5-fluorouracil, 5-fluorouridine, mitomycin-C, doxorubicin, and zotarolimus. Other cancer therapeutics include inhibitors of matrix metalloproteinases such as marimastat, growth factor antagonists, signal transduction inhibitors and protein kinase C inhibitors.

In some embodiments, the anti-cancer therapy comprises radiation therapy. In some embodiments, anti-cancer therapy comprises surgery to remove a tumour.

In some embodiments, the anti-cancer treatment comprises the administration of a chemotherapeutic drug selected from the group consisting of fluoropyrimidine (e.g., 5-FU), oxaliplatin, CPT-11, (e.g., irinotecan) a platinum drug or an anti EGFR antibody, such as the cetuximab antibody or a combination of such therapies, alone or in combination with surgical resection of the tumor. In yet a further aspect, the treatment comprises radiation therapy and/or surgical resection of the tumor masses. In one embodiment, the present invention encompasses administering to a subject identified as having, or increased risk of developing an anti-cancer combination therapy where combinations of anti-cancer agents are used, such as for example Taxol, cyclophosphamide, cisplatin, gancyclovir and the like. Anti-cancer therapies are well known in the art and are encompassed for use in the methods of the present invention. Chemotherapy includes, but is not limited to an alkylating agent, mitotic inhibitor, antibiotic, or antimetabolite, anti-angiogenic agents etc. The chemotherapy can comprise administration of CPT-11, temozolomide, or a platin compound. Radiotherapy can include, for example, x-ray irradiation, UV-irradiation, γ-irradiation, or microwaves.

The term “chemotherapeutic agent” or “chemotherapy agent” are used interchangeably herein and refers to an agent that can be used in the treatment of cancers and neoplasms, for example brain cancers and gliomas and that is capable of treating such a disorder. In some embodiments, a chemotherapeutic agent can be in the form of a prodrug which can be activated to a cytotoxic form. Chemotherapeutic agents are commonly known by persons of ordinary skill in the art and are encompassed for use in the present invention. For example, chemotherapeutic drugs for the treatment of tumors and gliomas include, but are not limited to: temozolomide (Temodar), procarbazine (Matulane), and lomustine (CCNU). Chemotherapy given intravenously (by IV, via needle inserted into a vein) includes vincristine (Oncovin or Vincasar PFS), cisplatin (Platinol), carmustine (BCNU, BiCNU), and carboplatin (Paraplatin), Mexotrexate (Rheumatrex orTrexall), irinotecan (CPT-11); erlotinib; oxalipatin; anthracycline-idarubicin and daunorubicin; doxorubicin; alkylating agents such as melphalan and chlorambucil; cisplatinum, methotrexate, and alkaloids such as vindesine and vinblastine.

Alkylating agents are polyfunctional compounds that have the ability to substitute alkyl groups for hydrogen ions. Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). These compounds react with phosphate, amino, hydroxyl, sulfihydryl, carboxyl, and imidazole groups. Under physiological conditions, these drugs ionize and produce positively charged ion that attach to susceptible nucleic acids and proteins, leading to cell cycle arrest and/or cell death. Combination therapy including a compound described herein and an alkylating agent can have therapeutic synergistic effects on cancer and reduce sides affects associated with these chemotherapeutic agents.

Some examples of anti-VEGF agents include bevacizumab (Avastin™), VEGF Trap, CP-547,632, AG13736, AG28262, SU5416, SU11248, SU6668, ZD-6474, ZD4190, CEP-7055, PKC 412, AEE788, AZD-2171, sorafenib, vatalanib, pegaptanib octasodium, IM862, DC101, angiozyme, Sirna-027, caplostatin, neovastat, ranibizumab, thalidomide, and AGA-1470, a synthetic analog of fumagillin (alternate names: Amebacilin, Fugillin, Fumadil B, Fumadil) (A. G. Scientific, catalog #F1028), an angio-inhibitory compound secreted by Aspergillus fumigates.

As used herein the term “anti-VEGF agent” refers to any compound or agent that produces a direct effect on the signaling pathways that promote growth, proliferation and survival of a cell by inhibiting the function of the VEGF protein, including inhibiting the function of VEGF receptor proteins. Exemplary VEGF inhibitors, i.e., anti-VEGF agents, include for example, AVASTIN® (bevacizumab), an anti-VEGF monoclonal antibody of Genentech, Inc. of South San Francisco, Calif., VEGF Trap (Regeneron/Aventis). Additional VEGF inhibitors include CP-547,632 (3-(4Bromo-2,6-difluoro-benzyloxy)-5-[3-(4-pyrrolidin 1-yl-butyl)-ureido]-isothiazole-4-carboxylic acid amide hydrochloride; Pfizer Inc., NY), AG13736, AG28262 (Pfizer Inc.), SU5416, SU11248, & SU6668 (formerly Sugen Inc., now Pfizer, New York, N.Y.), ZD-6474 (AstraZeneca), ZD4190 which inhibits VEGF-R2 and -R1 (AstraZeneca), CEP-7055 (Cephalon Inc., Frazer, Pa.), PKC 412 (Novartis), AEE788 (Novartis), AZD-2171), NEXAVAR® (BAY 43-9006, sorafenib; Bayer Pharmaceuticals and Onyx Pharmaceuticals), vatalanib (also known as PTK-787, ZK-222584: Novartis & Schering: AG), MACUGEN® (pegaptanib octasodium, NX-1838, EYE-001, Pfizer Inc./Gilead/Eyetech), IM862 (glufanide disodium, Cytran Inc. of Kirkland, Wash., USA), VEGFR2-selective monoclonal antibody DC101 (ImClone Systems, Inc.), angiozyme, a synthetic ribozyme from Ribozyme (Boulder, Colo.) and Chiron (Emeryville, Calif.), Sirna-027 (an siRNA-based VEGFR1 inhibitor, Sirna Therapeutics, San Francisco, Calif.) Caplostatin, soluble ectodomains of the VEGF receptors, Neovastat (AEterna Zentaris Inc; Quebec City, Calif.) and combinations thereof.

In some embodiments, the anti-cancer agent is selected from the group consisting of 5-fluorouracil, actinomycin D, adriamycin, Aldesleukin, Alemtuzumab, alitretinoin, alkaloids, alkylating agents, allopurinol, altretamine, amifostine, anastrozole, angiostatin, anthracycline-idarubicin and daunorubicin, anti EGFR antibody, anti-angiogenic agent, antibiotic agent, antimetabolite agent, arsenic trioxide, Asparaginase, azathioprine, bexarotene capsules, bexarotene gel, BiCNU), bleomycin, busulfan intravenous, busulfanoral, calusterone, capecitabine, carboplatin, carmustine, carmustine (BCNU), carmustine with Polifeprosan, celecoxib, chlorambucil, cisplatin, cisplatin, cisplatinum, cladribine, CPT-11, cyclophosphamide, cyclosporine, cytarabine, dacarbazine, dactinomycin, Darbepoetin alfa, daunomycin, daunorubicin, daunorubicin liposomal, Denileukin diftitox, dexrazoxane, docetaxel, doxorubicin, doxorubicin liposomal, Dromostanolone propionate, Elliott's B Solution, endostatin, epirubicin, Epoetin alfa estramustine, epothilones, erlotinib, etoposide phosphate, exemestane, Filgrastim, floxuridine (intraarterial), fludarabine, fulvestrant, gancyclovir, gemtuzumab ozogamicin, germicitibine, goserelin acetate, growth factor antagonists, halofuginone, hydroxyurea, Ibritumomab Tiuxetan, idarubicin, ifosfamide, imatinib mesylate, inhibitors of matrix metalloproteinases, Interferon alfa-2a, Interferon alfa-2b, irinotecan (CPT-11), letrozole, leucovorin, levamisole, LOddC, lomustine (CCNU), marimastat, mechlorethamine (nitrogenmustard), megestrol acetate, melphalan (L-PAM), mercaptopurine (6-MP), mesna, methotrexate, methoxsalen, mithramycin, mitomycin C, mitotane, mitotic inhibitor, mitoxantrone, mutamycin, nandrolone phenpropionate, Nofetumomab, Oprelvekin, oxalipatin, paclitaxel, pamidronate, pegademase, Pegaspargase, Pegfilgrastim, pentostatin, pipobroman, plasmin, plicamycin, porfimer sodium, procarbazine, protein kinase C inhibitors, quinacrine, Rasburicase, Rituximab, Sargramostim, signal transduction inhibitors, streptozocin, talbuvidine (LDT), talc, tamoxifen, temozolomide, teniposide (VM-26), testolactone, thioguanine (6-TG), thiotepa, thymidine kinase inhibitors, topotecan, toremifene, Tositumomab, trapidil, Trastuzumab, tretinoin (ATRA), Uracil Mustard, valrubicin, valtorcitabine (monoval LDC), vinblastine, vincristine (Oncovin or Vincasar PFS), vindesine, vinorelbine, zoledronate, zotarolimus, and any combinations thereof.

It is to be understood that the platelet decoys and methods disclosed herein can be used as a direct therapy (decoys alone), co-therapy (other drugs and/or surgery), and as drug delivery approach. For example, there are many types of cancers that result in DIC due to increased platelet activation/counts and the Decoys could be used in the same treatment regimen (depending on stability, ½ life, dose) with a chemotherapy agent in order to reduce the morbidity associated with DIC while treating the primary tumor. Decoys can also be used as a co-treatment with oncology surgeries where the tumor is being resected in order to prevent DIC or scavenge free tumor cells. The Decoys can also be used to directly treat DIC (or other coagulapathies) or to treat/prevent metastasis. The Decoys could also deliver a drug payload to treat metastasis or DIC associated with trauma, DVT, sepsis or other cardiovascular diseases.

In addition, platelet Decoys and methods disclosed herein can also be used to overcome antiplatelet drug resistance. For an example of antiplatelet drug resistance see Garabedian T, Alam S. High residual platelet reactivity on clopidogrel: its significance and therapeutic challenges overcoming clopidogrel resistance. Cardiovasc Diagn Ther. 2013; 3(1):23-37. Epub 2013 Nov. 28. doi: 10.3978/j.issn.2223-3652.2013.02.06. PubMed PMID: 24282742; PubMed Central PMCID: PMC3839215).

In some embodiments, from about 30,000 to about 60,000 platelets per microliter of blood can be supplemented in a subject with a single standard platelet transfusion. In some embodiments, platelet decoys could be identified in the spleen and the liver as “aged” platelets and be destroyed. This could be addressed by (i) increasing the dose used but also (ii) using an alternative procedure to obtain decoys which are not identified by the liver, (iii) transfecting parent platelets before preparing decoys so that they express anti-apoptotic proteins or repress apoptotic ones.

For administration to a subject, the platelet decoys described herein can be provided in pharmaceutically acceptable (e.g., sterile) compositions. Accordingly, another aspect described herein is a pharmaceutical composition comprising a platelet decoy and a pharmaceutically acceptable carrier. These pharmaceutically acceptable compositions comprise an effective amount of the platelet decoys described herein, formulated together with one or more pharmaceutically acceptable carriers (additives) and/or diluents. As described in detail below, the pharmaceutical compositions of the present disclosure can be specifically formulated for intravenous administration of the suspension (e.g., bolus or infusion). Additionally, the composition can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, content of all of which is herein incorporated by reference.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (or human) administration, it will be understood that compositions should meet sterility, pyrogenicity, general safety and purity standards as required by FDA Office of Biological Standards.

As used herein, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable carrier” or the like are used interchangeably herein.

Exemplary embodiments of the various aspects disclosed herein can be described by one or more of the numbered paragraphs:
1. A modified platelet cell, wherein the modified platelet is substantially free of at least one membrane lipid or molecular component present in an unmodified platelet cell, and wherein the modified platelet cell is capable of binding to a cell ligand or platelet receptor substrate but does not promote cell aggregation and/or stimulate blood coagulation.
2. The modified platelet cell of paragraph 1, wherein said modified platelet cell retains at least one receptor selected from the group consisting of any of the following receptors: glycoprotein (GP) IIb/IIIa, GP Ib-IX-V, CD9, GPVI, CLEC-2, P2 receptors, αvβ3, GPIa-IIa, α5β1, α6β1, toll-like receptors, protease activation receptors, PGD₂, PGE₂, PAF receptors, Lysophosphatidic acid receptors, Sphingosine-1-phosphate receptors, chemokine receptors, JAMs, ICAm-2, PECAm-1, G6B, CD47, ESAM, TLT-1, CD62P, CD72, CD93, CLEC-2, C-type lectine receptors, CD63, CD84, CD151, GPI anchored proteins, glycosaminoglycan-carrying receptors, CD110, leptin receptor, Tie-1 receptor, insulin receptor, PDGF receptor, Gas6 receptors, PEAR1, Eph kinases, CD36, C1q receptor, Cd46, Serotonin Reuptake receptor, LAMP-1, LAMP-2, CD40L, CD154, PSGL-1, P2X₁, tight junction receptors, TNF receptor, Semaphorin 3A receptors, CD100, PPARγ, CD147, glutamate receptors, liver x receptors, galectin receptors.
3. The modified platelet cell of paragraph 1 or 2, wherein the modified platelet cell binds directly or indirectly a tumor cell, a modified or unmodified platelet cell, an endothelial cell, white blood cells, extracellular matrix, coagulation proteins (e.g., fibrino) or any combinations thereof.
4. The modified platelet cell of any of paragraphs 1-3, wherein the platelet is substantially free of at least one component or component functionality required for platelet activation, cell aggregation or blood coagulation.
5. The modified platelet cell of paragraph 4, wherein said at least one component is a membrane lipid or molecular component.
6. The modified platelet cell of paragraph 4 or 5, wherein said at least one component is a membrane or cytosolic component required for platelet adhesion, activation and/or aggregation.
7. The modified platelet cell of any of paragraphs 1-6, wherein modified platelet cell is prepared from a platelet cell obtained from a subject.
8. The modified platelet cell of paragraph 7, wherein said subject is a healthy donor or is in need of treating, preventing or inhibiting a disease or disorder where platelet activation, aggregation and/or adhesion contributes to the pathology or symptomology of the disease.
9. The modified platelet cell of paragraph 7 or 8, wherein the subject is in need of treating, preventing or inhibiting tumor cell metastasis.
10. The modified platelet cell of paragraph 7 or 8, wherein the subject is in need of treating, preventing or inhibiting a coagulopathy.
11. The modified platelet cell of any of paragraphs 1-10, wherein the modified platelet cell is used as an anti-coagulant agent to prevent platelet-mediated blood coagulation.
12. The modified platelet cell of any of paragraphs 1-11, wherein the modified platelet cell is used to deliver an anti-coagulant or a fibrinolytic agent to prevent blood coagulation or induce fibrinolysis.
13. The modified platelet cell paragraph 12, wherein the anti-coagulant or fibrinolytic agent is encapsulated in or coated on the modified platelet cell.
14. The modified platelet cell of paragraph 13, wherein the anti-coagulant agent is selected from the group consisting of Warfarin, Acenocoumarol, Phenindione, Dabigatran, Apixaban, Rivaroxaban, and any combinations thereof
15. The modified platelet cell paragraph 13, wherein the fibrinolytic agent is a tissue-type plasminogen activator (t-PA), streptokinase (SK), prourokinase, urokinase (uPA), alteplase (also known as Activase®, Genentech, Inc.), reteplase (also known as r-PA or RETAVASE®, Centocor, Inc.), tenecteplase (also known as TNK™, Genentech, Inc.), STREPTASE® (AstraZeneca, LP), lanoteplase (Bristol-Myers Squibb Company), monteplase (Eisai Company, Ltd.), saruplase (also known as r-scu-PA and Rescupase™, Grunenthal GmbH, Corp.), staphylokinase, and anisoylated plasminogen-streptokinase activator complex (also known as APSAC, Anistreplase and Eminase®, SmithKline Beecham Corp.), and any combinations thereof
16. The modified platelet cell of any of paragraphs 1-15, wherein the modified platelet cell comprises an inhibitor of platelet activation (anti-platelet drug) encapsulated in or coated on the modified platelet cell.
17. The modified platelet of paragraph 16, wherein the anti-platelet drug is selected from the group consisting of Ticlopidine, Clopidogrel, Prasugrel, Ticagrelor, Cangrelor, Elinogrel, Abciximab, Eptifibatide, Tirofiban, Dipyridamole, Cilostazol, Aspirin, Aggrenox, Ap4A derivatives, rCD39, Arthropod apyrase, rHuman Apyrase, terutroban, Ridogrel, Terbogrel, Picotamide, NCX-4016, Orofiban, Lotrafiban, Sibrafiban, Zemilofiban, RUC-1, Vorapaxar, Atopaxar, Anti-PAR4, Anti-PAR1, LIBS-Tap, 6B4-F_{ab}, H6B4-F_{ab}, Crotalin, Mamushigin, VCL, AjvW-2, Aurin Tricarboxylic acid, ARC1779, Kistomin, mF1232, cF1232, EMS-16, CTRP-1, Revacept, TGX221, Ketanserin, Sarpogrelate, APD791, DG-041, soluble Ax1 domains, Arp2/3 Antidody, rPSGL-Ig, PSI-697, PSI-421, CD40 antibody, steroidal glycosides, and any combinations thereof
18. The modified platelet cell of any of paragraphs 1-17, wherein the modified platelet further comprises an anti-cancer agent encapsulated in or coated on the modified platelet cell.
19. The modified platelet of paragraph 18, wherein the anti-cancer agent is selected from the group consisting of 5-fluorouracil, actinomycin D, adriamycin, Aldesleukin, Alemtuzumab, alitretinoin, alkaloids, alkylating agents, allopurinol, altretamine, amifostine, anastrozole, angiostatin, anthracycline-idarubicin and daunorubicin, anti EGFR antibody, anti-angiogenic agent, antibiotic agent, antimetabolite agent, arsenic trioxide, Asparaginase, azathioprine, bexarotene capsules, bexarotene gel, BiCNU), bleomycin, busulfan intravenous, busulfanoral, calusterone, capecitabine, carboplatin, carmustine, carmustine (BCNU), carmustine with Polifeprosan, celecoxib, chlorambucil, cisplatin, cisplatin, cisplatinum, cladribine, CPT-11, cyclophosphamide, cyclosporine, cytarabine, dacarbazine, dactinomycin, Darbepoetin alfa, daunomycin, daunorubicin, daunorubicin liposomal, Denileukin diftitox, dexrazoxane, docetaxel, doxorubicin, doxorubicin liposomal, Dromostanolone propionate, Elliott's B Solution, endostatin, epirubicin, Epoetin alfa estramustine, epothilones, erlotinib, etoposide phosphate, exemestane, Filgrastim, floxuridine (intraarterial), fludarabine, fulvestrant, gancyclovir, gemtuzumab ozogamicin, germicitibine, goserelin acetate, growth factor antagonists, halofuginone, hydroxyurea, Ibritumomab Tiuxetan, idarubicin, ifosfamide, imatinib mesylate, inhibitors of matrix metalloproteinases, Interferon alfa-2a, Interferon alfa-2b, irinotecan (CPT-11), letrozole, leucovorin, levamisole, LOddC, lomustine (CCNU), marimastat, mechlorethamine (nitrogenmustard), megestrol acetate, melphalan (L-PAM), mercaptopurine (6-MP), mesna, methotrexate, methoxsalen, mithramycin, mitomycin C, mitotane, mitotic inhibitor, mitoxantrone, mutamycin, nandrolone phenpropionate, Nofetumomab, Oprelvekin, oxalipatin, paclitaxel, pamidronate, pegademase, Pegaspargase, Pegfilgrastim, pentostatin, pipobroman, plasmin, plicamycin, porfimer sodium, procarbazine, protein kinase C inhibitors, quinacrine, Rasburicase, Rituximab, Sargramostim, signal transduction inhibitors, streptozocin, talbuvidine (LDT), talc, tamoxifen, temozolomide, teniposide (VM-26), testolactone, thioguanine (6-TG), thiotepa, thymidine kinase inhibitors, topotecan, toremifene, Tositumomab, trapidil, Trastuzumab, tretinoin (ATRA), Uracil Mustard, valrubicin, valtorcitabine (monoval LDC), vinblastine, vincristine (Oncovin or Vincasar PFS), vindesine, vinorelbine, zoledronate, zotarolimus, anti-angiogenic protein, and any combinations thereof
20. The modified platelet cell of any of paragraphs 1-19, wherein the modified platelet cell further comprises an imaging agent encapsulated in or coated on the modified platelet cell.
21. The modified platelet cell of paragraph 20, wherein the imaging agent is selected from the group consisting of wherein the imaging agent is selected from the group consisting of Alexa Fluor® dyes (InvitrogenCorp.; Carlsbad, Calif.); fluorescein; fluorescein isothiocyanate (FITC); Oregon Green™; tetrarhodamine isothiocynate (TRITC), 5-carboxyfluorescein (FAM); 2′7′-dimethoxy-4′5′-dichloro-6-carboxyfluorescein (JOE); tetrachlorofluorescein (TET); 6-carboxyrhodamine (R6G); N,N,N,N′-tetramefhyl-6-carboxyrhodamine (TAMRA); 6-carboxy-X-rhodamine (ROX); naphthylamine dyes having an amino group in the alpha or beta position; coumarins and derivatives thereof; acridines and derivatives thereof; N-(p(2-benzoxazolyl)phenyl)maleimide; cyanines and derivatives thereof; 1H,5H,11H, 15H-Xantheno[2,3,4-ij:5,6,7-i′j′]diquinolizin-18-ium, 9-[2(or 4)-[[[6-[2,5-dioxo-1-pyrrolidinyl)oxy]-6-oxohexyl] amino]sulfonyl]-4(or 2)-sulfophenyl]-2,3,6,7,12,13,16,17octahydro-inner salt (TR or Texas Red); BODIPY™ dyes; benzoxadiazoles; stilbenes; pyrenes; fluorescent proteins and variants thereof; radioisotopes; paramagnetic metal ions; X-ray absorbers; and any combinations thereof
22. The modified platelet cell of any of paragraphs 1-21, wherein the modified platelet cell expresses at least one anti-apoptotic molecule at an increased expression level or amount relative to an unmodified or normal platelet cell.
23. The modified platelet cell of any of paragraphs 1-22, wherein the modified platelet cell expresses an exogenous gene encoding an anti-apoptotic molecule or repress an apoptotic protein.
24. The modified platelet cell of any of paragraphs 1-23, wherein the modified platelet cell comprises an inhibitor of a pro-apoptotic molecule or inhibitor of a molecule that promotes cell death.
25. The modified platelet cell of any of paragraphs 1-24, wherein the modified platelet cell expresses at least one pro-apoptotic molecule or a molecule that promotes cell death at decreased/reduced expression level or amount.
26. A method comprising administering to a subject a composition comprising: (i) a modified platelet cell of any of paragraphs 1-24.
27. The method of paragraph 26, wherein the subject is in need of treating, preventing or inhibiting a disease or disorder when platelet activation, aggregation and/or adhesion contributes to the pathology or symptomology of the disease.
28. The method of paragraph 26 or 27, wherein the subject is in need of: (i) treating, preventing or inhibiting tumor cell metastasis or tumor cell interactions with platelets in the circulation; (ii) enhancing, increasing, or stimulating fibrinolysis; (iii) treating, preventing or suppressing excessive platelet aggregation, blood coagulation or clotting disorders; (iv) enhancing, increasing, or stimulating clot formation at a platelet binding site; or (v) imaging platelet aggregation or blood clot formation or (vi) detecting any cells that platelet decoys can directly or indirectly bind.
29. The method of any of paragraphs 26-28, the method further comprising co-administering an anti-cancer therapy or anti-coagulant agent or fibrinolytic agent to the subject.
30. The method of paragraph 29, wherein said anti-cancer therapy is selected from the group consisting of radiotherapy, photodynamic therapy, surgery, chemotherapy, and any combinations thereof
31. The method of paragraph 29 or 30, wherein said anti-coagulant agent is selected from the group consisting of Warfarin, Acenocoumarol, Phenindione, Dabigatran, Apixaban, Rivaroxaban, and any combinations thereof
32. The method of any of paragraphs 29-31, wherein the fibrinolytic agent is a tissue-type plasminogen activator (t-PA), streptokinase (SK), prourokinase, urokinase (uPA), alteplase (also known as Activase®, Genentech, Inc.), reteplase (also known as r-PA or Retavase®, Centocor, Inc.), tenecteplase (also known as TNK™, Genentech, Inc.), Streptase® (AstraZeneca, LP), lanoteplase (Bristol-Myers Squibb Company), monteplase (Eisai Company, Ltd.), saruplase (also known as r-scu-PA and Rescupase™, Grunenthal GmbH, Corp.), staphylokinase, and anisoylated plasminogen-streptokinase activator complex (also known as APSAC, Anistreplase and Eminase®, SmithKline Beecham Corp.), and any combinations thereof
33. The method of any of paragraphs 27-32, wherein said disease or disorder is a coagulopathy.
34. A method of preparing a modified platelet cell, the method comprising: removing or extracting or modifying at least one membrane lipid or molecular component from a platelet, thereby obtaining a modified platelet cell that is capable of binding to a cell ligand or a platelet receptor substrate but does not undergo aggregation and/or promote blood coagulation.
35. The method of paragraph 34, wherein said removing or modifying comprises treating the platelets with detergent, high salt, ammonium hydroxide, and/or a fixative.
36. The method of paragraph 35, wherein said detergent is selected from the group consisting of Triton (Triton-X100 and other family members), Nonidet, octylphenol ethoxylates, nonyl phenoxypolyethoxylethanol (NP-40), octylphenoxypolyethoxyethanol (Nonidet P-40), Poloxamers, Spans, octenidine dihydrochloride, alkyltrimethylammonium salts, cetyl trimethylammonium bromide (CTAB) or hexadecyl trimethyl ammonium bromide, cetyl trimethylammonium chloride (CTAC)), cetylpyridinium chloride (CPC), benzalkonium chloride (BAC), benzethonium chloride (BZT), 5-Bromo-5-nitro-1,3-dioxane, dimethyldioctadecylammonium chloride, cetrimonium bromide, dioctadecyldimethylammonium bromide (DODAB), sulfonates, sultaines, betaines, lecithins, cetyl alcohol, stearyl alcohol, cetostearyl alcohol (consisting predominantly of cetyl and stearyl alcohols), oleyl alcohol, polyoxyethylene glycol alkyl ethers (Brij) of formula: CH₃—(CH₂)₁₀₋₁₆—(O—C₂H₄)₁₋₂₅—OH, polyoxypropylene glycol alkyl ethers of formula: CH₃—(CH₂)₁₀₋₁₆—(O—C₃H₆)₁₋₂₅—OH, glucoside alkyl ethers of formula: CH₃—(CH₂)₁₀₋₁₆—(O-Glucoside)₁₋₃-OH, polyoxyethylene glycol octylphenol ethers of formula: C₈H₁₇—(C₆H₄)—(O—C₂H₄)₁₋₂₅—OH, polyoxyethylene glycol alkylphenol ethers of formula: C₉H₁₉—(C₆H₄)—(O—C₂H₄)₁₋₂₅—OH, glycerol alkyl esters, polyoxyethylene glycol sorbitan alkyl esters, sorbitan alkyl esters, cocamide MEA; cocamide DEA, dodecyldimethylamine oxide, block copolymers of polyethylene glycol and polypropylene glycol, polyethoxylated tallow amine, and any combinations thereof
37. The method of any of paragraphs 34-36, wherein said treating the platelets is for a period of about less than about 60 minutes.
38. The method of paragraph 37, wherein said treating the platelets is for a period of about 10 seconds.
39. The method of any of paragraphs 34-38, wherein the method further comprises obtaining platelets from a subject.
40. The method of paragraph 39, wherein the subject is a healthy subject or the subject in need of treatment for a disease or disorder where platelet activation, aggregation, and/or adhesion contributes to the pathology or symptomology of the disease or disorder.
41. The method of any of paragraphs 34-40, further comprising inhibiting or reducing the expression level or amount in the platelet of a pro-apoptotic molecule or a molecule a molecule that promotes cell death before said extraction.
42. The method of any of paragraphs 34-41, further comprising increasing the expression or level of an anti-apoptotic molecule in the platelet.
43. The method of any of paragraphs 34-42, further comprising expressing an exogenous gene encoding an anti-apoptotic molecule in the platelet.

### SOME SELECTED DEFINITIONS

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used herein, the term “cancer” refers to an uncontrolled growth of cells that may interfere with the normal functioning of the bodily organs and systems. Cancers that migrate from their original location and seed vital organs can eventually lead to the death of the subject through the functional deterioration of the affected organs. A metastasis, a cancer cell or group of cancer cells, distinct from the primary tumor location resulting from the dissemination of cancer cells from the primary tumor to other parts of the body. At the time of diagnosis of the primary tumor mass, the subject may be monitored for the presence of in transit metastases, e.g., cancer cells in the process of dissemination. As used herein, the term cancer, includes, but is not limited to the following types of cancer, breast cancer, biliary tract cancer, bladder cancer, brain cancer including Glioblastomas and medulloblastomas; cervical cancer; choriocarcinoma; colon cancer; endometrial cancer; esophageal cancer, gastric cancer; hematological neoplasms including acute lymphocytic and myelogenous leukemia; T-cell acute lymphoblastic leukemia/lymphoma; hairy cell leukemia; chronic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma, Wilms tumor. Examples of cancer include but are not limited to, carcinoma, including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, Glioblastoma, cervical cancer, ovarian cancer, liver cancer such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. Other cancers will be known to the artisan.

As used herein, the term “cancer” includes, but is not limited to, solid tumors and blood born tumors. The term cancer refers to disease of skin, tissues, organs, bone, cartilage, blood and vessels. The term “cancer” further encompasses primary and metastatic cancers. Examples of cancers that can be treated with the compounds of the invention include, but are not limited to, carcinoma, including that of the bladder, breast, colon, kidney, lung, ovary, pancreas, stomach, cervix, thyroid, and skin, including squamous cell carcinoma; hematopoietic tumors of lymphoid lineage, including, but not limited to, leukemia, acute lymphocytic leukemia, acute lymphoblastic leukemia, B-cell lymphoma, T-cell lymphoma, Hodgkins lymphoma, non-Hodgkins lymphoma, hairy cell lymphoma, and Burketts lymphoma; hematopoietic tumors of myeloid lineage including, but not limited to, acute and chronic myelogenous leukemias and promyelocytic leukemia; tumors of mesenchymal origin including, but not limited to, fibrosarcoma, rhabdomyosarcoma, and osteosarcoma; other tumors including melanoma, seminoma, tetratocarcinoma, neuroblastoma, and glioma; tumors of the central and peripheral nervous system including, but not limited to, astrocytoma, neuroblastoma, glioma, and schwannomas; and other tumors including, but not limited to, xenoderma, pigmentosum, keratoactanthoma, thyroid follicular cancer, and teratocarcinoma. The compounds of the invention are useful for treating patients who have been previously treated for cancer, as well as those who have not previously been treated for cancer. Indeed, the methods and compositions of this invention can be used in first-line and second-line cancer treatments.

In some embodiments, the cancer or metastasis is selected from the group consisting of platinum susceptible or resistant tumors including breast, head and neck, ovarian, testicular, pancreatic, oral-esophageal, gastrointestinal, liver, gall bladder, lung, melanoma, skin cancer, sarcomas, blood cancers, brain tumors including glioblastomas, and tumors of neuroectodermal origin.

As used herein, the term “precancerous condition” has its ordinary meaning, i.e., an unregulated growth without metastasis, and includes various forms of hyperplasia and benign hypertrophy. Accordingly, a “precancerous condition” is a disease, syndrome, or finding that, if left untreated, can lead to cancer. It is a generalized state associated with a significantly increased risk of cancer. Premalignant lesion is a morphologically altered tissue in which cancer is more likely to occur than its apparently normal counterpart. Examples of pre-malignant conditions include, but are not limited to, oral leukoplakia, actinic keratosis (solar keratosis), Barrett's esophagus, atrophic gastritis, benign hyperplasia of the prostate, precancerous polyps of the colon or rectum, gastric epithelial dysplasia, adenomatous dysplasia, hereditary nonpolyposis colon cancer syndrome (HNPCC), bladder dysplasia, precancerous cervical conditions, and cervical dysplasia.

Generally, the term “treatment” or “treating” is defined as the application or administration of a therapeutic agent to a patient, or application or administration of a therapeutic agent to an isolated tissue or cell line from a patient, said patient having a disease, a symptom of disease or a predisposition toward a disease, with the purpose to cure, heal, alleviate, relieve, alter, remedy, ameliorate, improve or affect the disease, the symptoms of disease or the predisposition toward disease. Thus, treating can include suppressing, inhibiting, preventing, treating, or a combination thereof. Treating refers, inter alia, to increasing time to sustained progression, expediting remission, inducing remission, augmenting remission, speeding recovery, increasing efficacy of or decreasing resistance to alternative therapeutics, or a combination thereof. “Suppressing” or “inhibiting”, refers, inter alia, to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. In one embodiment the symptoms are primary, while in another embodiment symptoms are secondary. “Primary” refers to a symptom that is a direct result of a disorder; while, secondary refers to a symptom that is derived from or consequent to a primary cause. Symptoms may be any manifestation of a disease or pathological condition.

Accordingly, as used herein, the term “treatment” or “treating” includes: (i) preventing the disease from occurring in a subject which may be predisposed to the disease but does not yet experience or display the pathology or symptomatology of the disease; (ii) inhibiting the disease in an subject that is experiencing or displaying the pathology or symptomatology of the diseased (i.e., arresting further development of the pathology and/or symptomatology); or (iii) ameliorating the disease in a subject that is experiencing or displaying the pathology or symptomatology of the diseased (i.e., reversing the pathology and/or symptomatology). In one embodiment, the symptoms of a disease or disorder are alleviated by at least 5%, at least 10%, at least 20%, at least 30%, at least 40%, or at least 50%. A complete amelioration of the symptoms of the disease or disorder is not required.

Efficacy of treatment is determined in association with any known method for diagnosing the disorder. Alleviation of one or more symptoms of the disorder indicates that the compound confers a clinical benefit.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

Preferably, the subject is a mammal. The mammal can be a human, non-human primate, mouse, rat, dog, cat, horse, or cow, but are not limited to these examples. Mammals other than humans can be advantageously used as subjects that represent animal models of human diseases and disorders. In addition, compounds, compositions and methods described herein can be used to treat domesticated animals and/or pets.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of a composition into a subject by a method or route which results in at least partial localization of the composition at a desired site such that desired effect is produced. Administration can be by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

The phrase “therapeutically-effective amount” as used herein means that amount of a compound, material, or composition comprising a compound described herein which is effective for producing some desired therapeutic effect in at least a sub-population of cells in a subject at a reasonable benefit/risk ratio applicable to any medical treatment. Thus, “therapeutically effective amount” means that amount which, when administered to a subject for treating a disease, is sufficient to effect such treatment for the disease.

Determination of an effective amount is well within the capability of those skilled in the art. Generally, the actual effective amount can vary with the specific compound, the use or application technique, the desired effect, the duration of the effect and side effects, the subject's history, age, condition, sex, as well as the severity and type of the medical condition in the subject, and administration of other pharmaceutically active agents. Accordingly, an effective dose of compound or composition is an amount sufficient to produce at least some desired therapeutic effect in a subject.

The data obtained in vitro and in animal studies can be used in formulating a range of dosage for use in humans. The dosage of such compounds lies preferably within a range of circulating concentrations that include the IC50 with little or no toxicity. The dosage may vary within this range depending upon the dosage form employed and the route of use or administration utilized.

The effective dose can be estimated initially from the in vitro assays. A dose can be formulated in animal models to achieve a circulating plasma concentration range that includes the IC50 (i.e., the concentration of the therapeutic which achieves a half-maximal inhibition of symptoms) as determined in cell culture. The effect of any particular dosage can be monitored by a suitable bioassay.

The disclosure is further illustrated by the following examples which should not be construed as limiting. The examples are illustrative only, and are not intended to limit, in any manner, any of the aspects described herein. The following examples do not in any way limit the invention.

### EXAMPLES

### Example 1

### Preparation of Platelet Decoys

The role of the GP IIb/IIIa receptor in the platelet-cancer cell interactions is well established (7, 15). Thus, a first objective of this study was to create Platelet Decoys by modifying platelets so that they retain the ability to bind to CTCs, but fail to activate normally.

In one example, Platelet Decoys were prepared as follow. Platelets were first extracted from fresh whole blood of healthy donors (either from pooled blood, or from individual patients for personalized therapy). To do so, whole blood was centrifuged to pellet red blood cells (RBCs) and leukocytes (290 g force, 15 min, no break applied); platelet rich plasma (PRP) was collected and centrifuged one more time (290 g, 15 min, no break applied) to eliminate virtually all of the remaining RBCs and leukocytes, and then the PRP was centrifuged again (990 g, 15 min, no break applied) to pellet platelets. The supernatant platelet-poor plasma (PPP) was stored, filtered through 0.2 μm pore size to purify it from any remaining platelets, and the platelet pellet was treated as follows to produce the Platelet Decoys

The platelet pellet was resuspended in 2.0 mL of PPP and 2.5 mL of extraction buffer (pH 7.4, 0.01 M HEPES, 0.05 M NaCl, 2.5 mM MgCl₂, 0.3 M sucrose) and 0.5 mL of Triton-X-100 was added to 0.1 M final concentration for 10 seconds before being centrifuged (990 g, 15 min, no break applied). The supernatant was discarded and the pellet was resuspended in PPP, washed by centrifugation and stored in PPP. This extraction process can be repeated one or more times before storage, if more complete inactivation of the decoys is desired. Also, other detergents, such as other octylphenol ethoxylates, nonyl phenoxypolyethoxylethanol (NP-40), octylphenoxypolyethoxyethanol (Nonidet P-40), could be used for this process. The time and concentration of detergent treatment can be modulated depending on the desired degree of reduction of platelet function as well. Alternative methods for obtaining Platelet Decoys could employ fixation of platelets using a short exposure to commercially available fixatives (such as aldehydes), or extraction using high salt, ammonium hydroxide or other buffers that have been shown to remove soluble lipids and molecular components from insoluble cellular and extracellular scaffolds, such as the cytoskeleton and extracellular matrix.

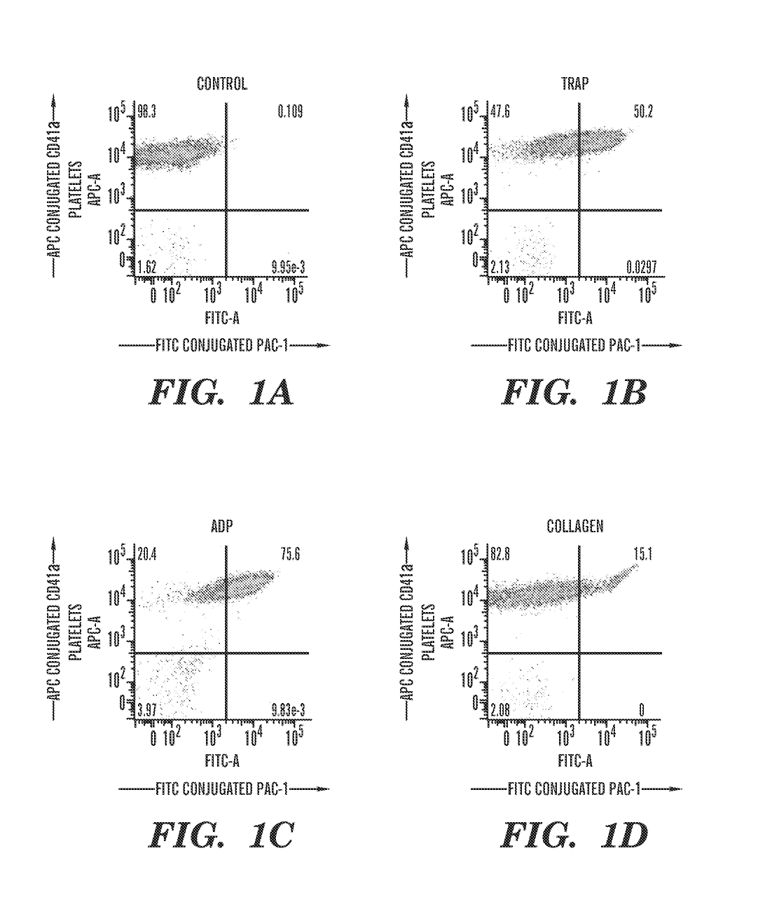

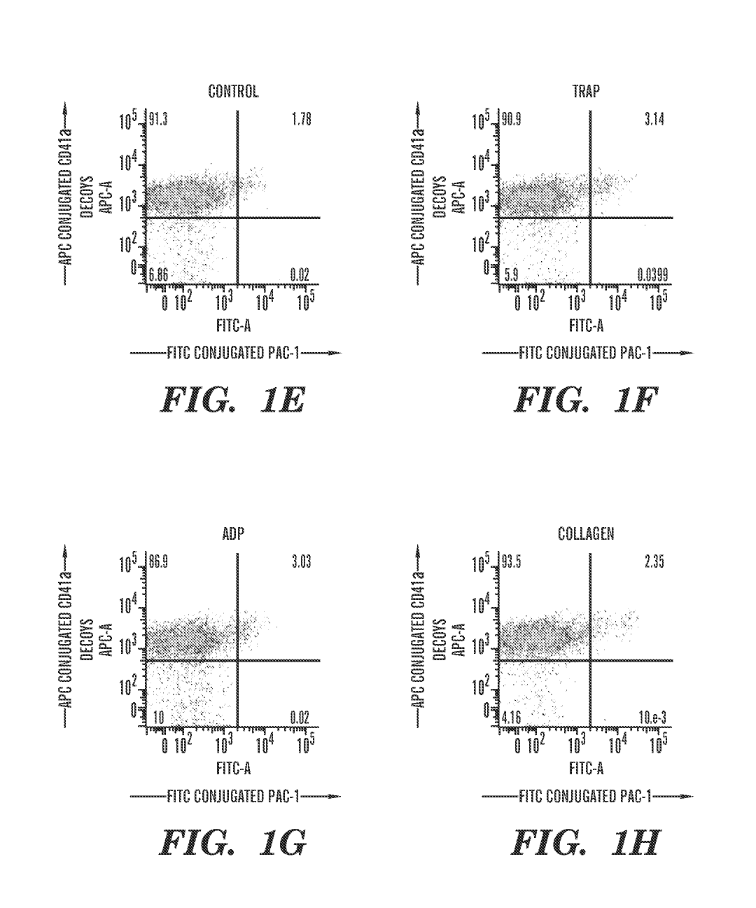

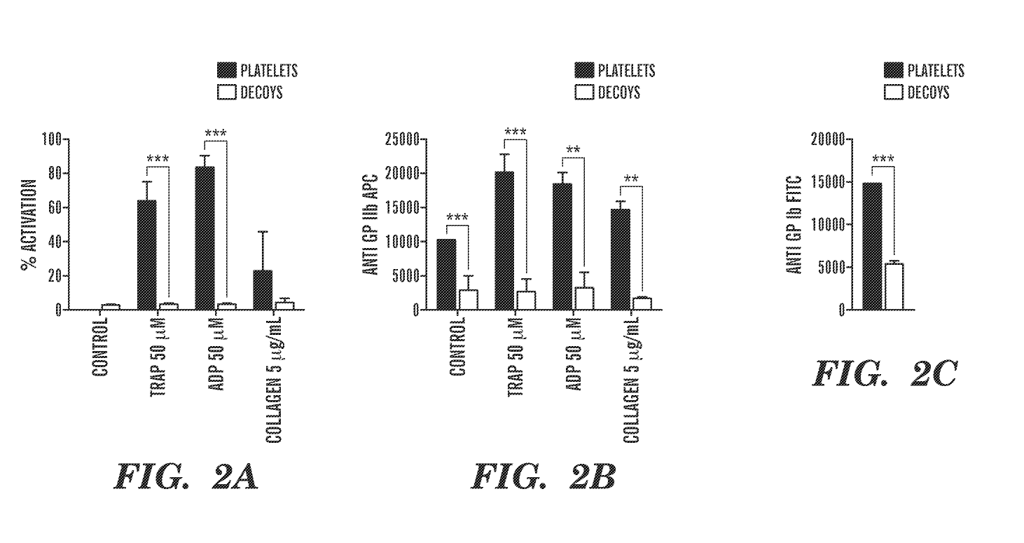

The data indicate that platelets become activated when stimulated with TRAP (thrombin receptor activating peptide), adenosine diphosphate (ADP) and collagen (FIGS. 1A-1D, and FIG. 2A), while the Platelet Decoys do not (FIGS. 1E-1H and FIG. 2A), even when these agonists were administered at a high concentration (TRAP and ADP: 50 μM, collagen: 5 μg/mL). Main receptor quantification of Platelet Decoys has been evaluated and compared to parent platelets (FIGS. 2B and 2C). It has been shown that CD41a and CD42b presence was significantly reduced in the case of Decoys compared to the platelet control (FIGS. 2B and 2C). Indeed, Platelet Decoys retain a degree of their CD41a (GP IIb/IIIa) receptor binding function (FIG. 2B), but agonist-induced CD41a recruitment at the Decoy surface was not observed under ADP stimuli, unlike platelets (FIG. 2B). Without wishing to be bound by a theory, Platelet Decoys can prevent formation of aggregates of tumor cells with platelets and fibrin, and thereby suppress metastasis. For example, CTCs coated by the modified platelets may not create big enough aggregates to induce embolism and capture in capillary microvessels, as compared to whole platelet-CTCs complexes. Thus, in the therapy context, platelets could be obtained from the patient, modified and injected again. In addition to providing a personalized therapy, this approach can also decrease the cancer patient's elevated platelet levels.

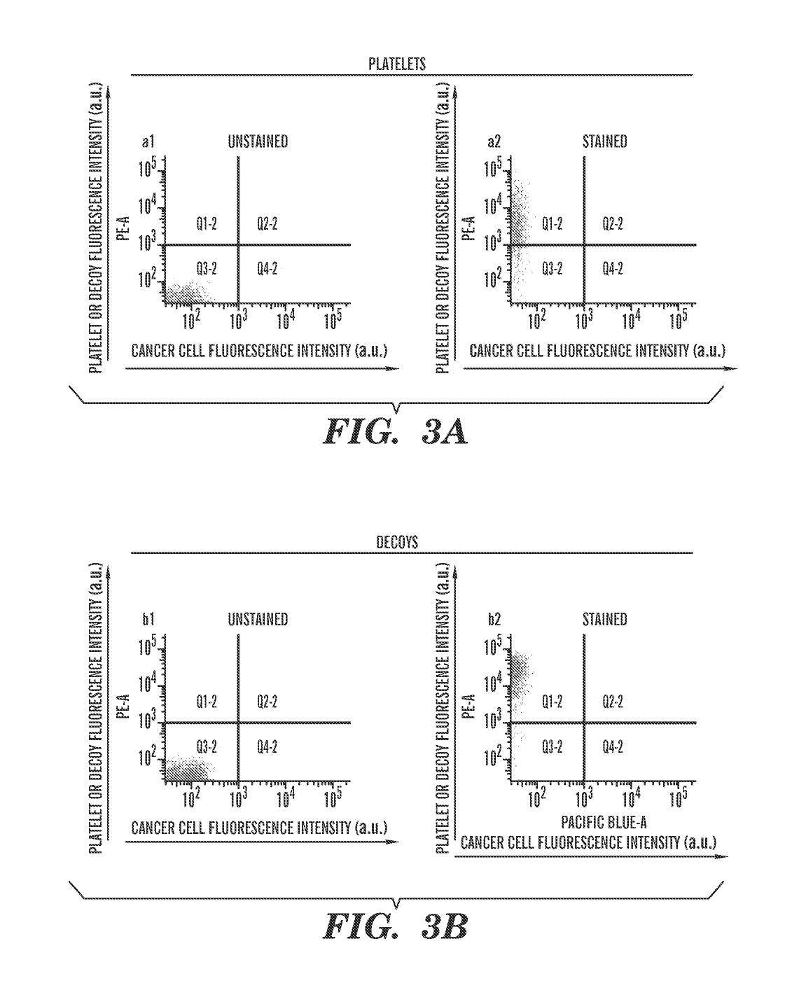

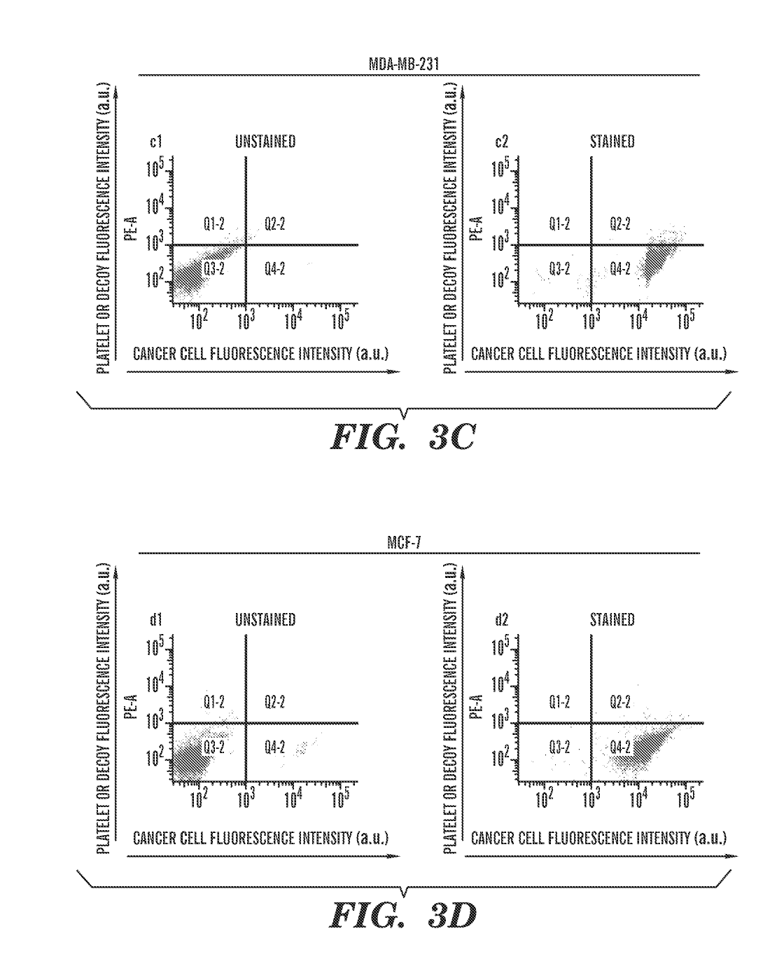

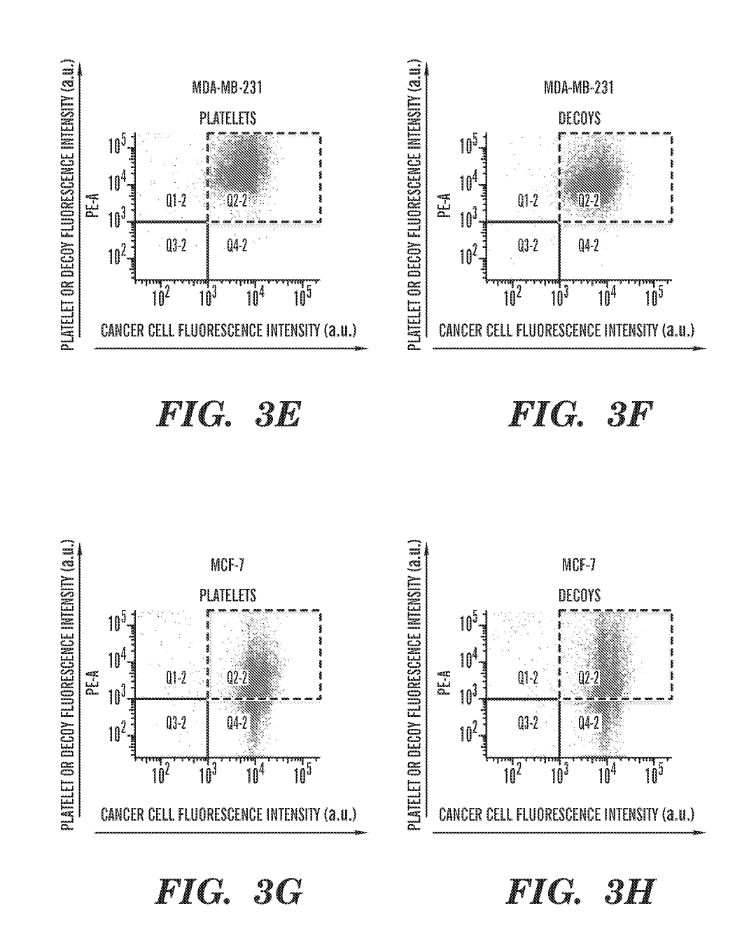

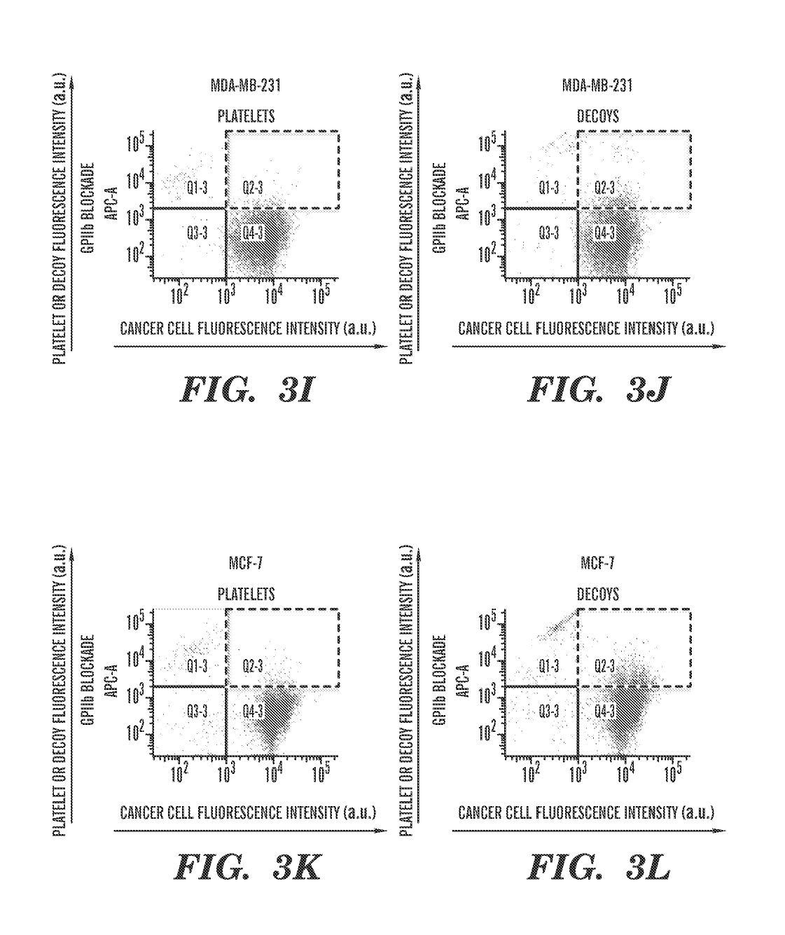

The ability of the Decoys to interact with cancer cells in plasma was also investigated and compared with platelet-cancer cell interactions. These studies revealed that the Platelet Decoys showed similar binding to both human MDA-MB-231 and MCF-7 breast cancer cells when compared with intact platelets (FIGS. 3A-3L), despite the reduced amount of CD41a receptor present at their surface.

It is well established that the activation response of platelets is a precursor of their aggregation. Following their activation characterization, we have compared the aggregation responses of platelets and Decoys. Platelet Decoys were found to be incapable of aggregation with a high concentration of agonists, e.g. 50 μM ADP (FIG. 4A) or 10 μg/mL collagen (FIG. 4B), while their platelet counterparts aggregated more than 50% in the same conditions. When decoys were co-incubation with platelets (P:D=1:0.25, 1:0.5, 1:1, 1:2), no obvious aggregation was observed over 20 min (FIG. 4C). Co-incubation of platelets and Decoys with agonists highlighted that Decoys decrease the magnitude of platelet aggregation (FIGS. 4D and 4E).

Collagen and fibrinogen coated surfaces have been used as an injury model to test the reactivity of the Decoys towards these surfaces over time and compared to normal platelets. Platelet aggregates were formed in a few minutes on both surfaces and increased over time. In addition, the effect was enhanced by concomitant incubation with 50 μM ADP. However, Decoys failed to aggregate on these surfaces both with prolonged exposure and ADP addition, corroborating the results obtained by aggregometry with a variety of agonists. These results altogether validate that Platelet Decoys are inert while facing chemical or contact stimuli normally leading to platelet activation and aggregation. Platelet Decoys are also able to immobilize proteins. For instance, recombinant human tPA (tissue Plasminogen Activator) labeled with the texas red dye has been retained at their surface. The contribution of active internalization to this process was excluded with a control group at 4° C. This model can be to a wide range of molecules (proteins, peptides, drugs, particles, radioelements) and use Decoys as a therapeutic or diagnostic carrier.

As described above, the inventors have demonstrated that the Platelet Decoys conserve their GP IIb/IIIa complex receptors, but that the activated conformation is not induced by stimulation with the agonists TRAP, ADP and collagen (FIGS. 1A-1H, 2A and 2B). The inventors have further shown that these Platelet Decoys can compete with natural platelets in terms of their ability to bind to tumor cells. After being extracted from whole blood of healthy donors, platelets were modified with a light detergent. After this step, the modified platelets (Platelet Decoys) conserved their GP IIb/IIIa complex receptors, but the activated conformation was not induced by stimulation with the agonist ADP.

Without wishing to be bound by a theory, a range of extraction procedures can be used to optimize retention of platelet adhesion while minimizing their ability to be activated by various chemical as well as physical stimuli. As shown herein, the platelet decoys can compete with natural platelets in terms of their ability to bind to tumor cells, and they display no or lower activation potential after contact with cancer cells than their platelet counterparts. This can be explored by (i) quantifying formation of aggregates containing tumor cells, platelets and fibrin, (ii) ADP release detection or (iii) PAC-1 binding evaluation. One can also evaluate whether modified platelets are robust under shear stress, and characterize effects on other receptors that are involved in tumor cell binding and platelet aggregation.

### REFERENCES FOR EXAMPLE 1

1. H. B. P. Pearson, N., In Metastatic Cancer: Integrated Organ System and Biological Approach, Landes Bioscience ed.; Jandial, R. H., K., Ed. 2012.
2. N. M. Bambace, C. E. Holmes, The platelet contribution to cancer progression. J Thromb Haemost 9, 237-249 (2011)
3. L. J. Gay, B. Felding-Habermann, Contribution of platelets to tumour metastasis. Nat Rev Cancer 11, 123-134 (2011)
4. G. L. Klement, T. T. Yip, F. Cassiola, L. Kikuchi, D. Cervi, V. Podust, J. E. Italiano, E. Wheatley, A. Abou-Slaybi, E. Bender, N. Almog, M. W. Kieran, J. Folkman, Platelets actively sequester angiogenesis regulators. Blood 113, 2835-2842 (2009)
5. E. Sierko, M. Z. Wojtukiewicz, Inhibition of platelet function: does it offer a chance of better cancer progression control? Semin Thromb Hemost 33, 712-721 (2007)
6. R. L. Stone, A. M. Nick, I. A. McNeish, F. Balkwill, H. D. Han, J. Bottsford-Miller, R. Rupairmoole, G. N. Armaiz-Pena, C. V. Pecot, J. Coward, M. T. Deavers, H. G. Vasquez, D. Urbauer, C. N. Landen, W. Hu, H. Gershenson, K. Matsuo, M. M. Shahzad, E. R. King, I. Tekedereli, B. Ozpolat, E. H. Ahn, V. K. Bond, R. Wang, A. F. Drew, F. Gushiken, D. Lamkin, K. Collins, K. DeGeest, S. K. Lutgendorf, W. Chiu, G. Lopez-Berestein, V. Afshar-Kharghan, A. K. Sood, Paraneoplastic thrombocytosis in ovarian cancer. N Engl J Med 366, 610-618 (2012)
7. P. Jurasz, D. Alonso-Escolano, M. W. Radomski, Platelet—cancer interactions: mechanisms and pharmacology of tumour cell-induced platelet aggregation. Br J Pharmacol 143, 819-826 (2004)
8. L. A. Coupland, B. H. Chong, C. R. Parish, Platelets and P-selectin control tumor cell metastasis in an organ-specific manner and independently of NK cells. Cancer Res 72, 4662-4671 (2012)
9. H. V. Naina, S. Harris, Paraneoplastic thrombocytosis in ovarian cancer. N Engl J Med 366, 1840; author reply 1840 (2012)
10. G. J. Gasic, T. B. Gasic, C. C. Stewart, Antimetastatic effects associated with platelet reduction. Proc Natl Acad Sci USA 61, 46-52 (1968)
11. M. Hejna, M. Raderer, C. C. Zielinski, Inhibition of metastases by anticoagulants. J Natl Cancer Inst 91, 22-36 (1999)
12. M. S. Cho, J. Bottsford-Miller, H. G. Vasquez, R. Stone, B. Zand, M. H. Kroll, A. K. Sood, V. Afshar-Kharghan, Platelets increase the proliferation of ovarian cancer cells. Blood 120, 4869-4872 (2012)
13. W. C. Aird, The hematologic system as a marker of organ dysfunction in sepsis. Mayo Clinic proceedings. Mayo Clinic 78, 869-881 (2003)
14. J. N. Katz, K. P. Kolappa, R. C. Becker, Beyond thrombosis: the versatile platelet in critical illness. Chest 139, 658-668 (2011)
15. H. Kitagawa, N. Yamamoto, K. Yamamoto, K. Tanoue, G. Kosaki, H. Yamazaki, Involvement of platelet membrane glycoprotein Ib and glycoprotein IIb/IIIa complex in thrombin-dependent and -independent platelet aggregations induced by tumor cells. Cancer Res 49, 537-541 (1989)

### Example 2

Platelets have been implicated in several diseases like ischemic heart disease, stroke, sepsis and cancer (1). These diseases have been acknowledged by the World Health Organization to be among the top 10 causes of mortality worldwide and there can be no bigger illustration of the powerful influence of platelets in human disease. This has led to a profusion of drugs that target platelet functions and the clotting process (2, 3). However, these drugs can often have serious side effects such as bleeding, they are not immediately effective and have interactions of their own (4). The work reported herein shows that the potential for development of new drug-free therapies to treat platelet related pathologies, using natural modified platelets (called “platelet decoys”). This new therapeutic tool could lead to a paradigm shift in current therapy for many diseases including the prevention of acute thrombosis and the prevention of cancer metastasis. Acute coronary syndromes (myocardial infarction and unstable angina) are a significant contributor to lifetime morbidity and mortality in the United States (5). Interventional procedures (i.e. Percutaneous Coronary Intervention—PCI) have acquired an increasing influence in the management of acute coronary syndromes. According to estimates, 954,000 PCI procedures were performed in 2010 (6). Though they have lead to improved outcomes, they also introduce a significantly increased risk of periprocedural thrombosis. This necessitates the use of concurrent anti-platelet agents with PCI. Current anti-platelet agents often fail to successfully combat this risk leading to the use of drug combinations, in spite of their much higher bleeding risks. Without wishing to be bound by a theory, platelet decoys can be utilized as a single, rapid, intravenous and carefully controlled anti-platelet agent in these situations. Their use in acute coronary syndromes can also be extended as an adjunct to fibrinolytic therapy. Platelets have a natural affinity to bind tPA (tissue Plasminogen Activator, a fibrinolytic agent) (7, 8) and tPA carrying decoys can be used to combine these therapies. Further, the platelet decoys can also be used beyond the prevention of thrombosis in high-risk patients.

In addition, the platelet decoys can be used for preventing cancer metastasis. Metastasis initiating Circulating Tumor Cells (CTCs) have been associated with a poor prognosis in breast cancer since the last 10 years, however no CTC targeting therapies have been developed till date (9). Platelet decoys can address this clinical need by interfering with platelet functions and denying the vital support provided by platelets to CTCs. This provides a novel platelet decoy based CTC targeting therapy which can be used as an adjuvant therapy in cancer such breast cancer.

Platelet Decoys disclosed herein are a completely novel technology that impact several diseases. Their fundamental concept is based on them being a natural imitator of platelet structure without their associated functional output. Modified platelets, derived from the patient or donors, provide a biological therapy that is non-immunogenic, non-toxic and drug-free. They can be used as therapeutics due to their intrinsic properties (anti-platelet effect). This approach using a detergent method to produce platelet decoys from parent platelets has not been reported in literature. Such a method of modifying platelet functionality (inhibiting activation and aggregation potential) has not been reported as well. Thus, the present disclosure is the first reported instance of utilizing a platelet imitating biological therapy to induce a competitive environment, leading to an inhibition of circulating platelet function. Without wishing to be bound by a theory, these properties are useful in preventing thrombosis in acute care situations such as during an emergency PCI procedure. In addition, their preserved complex surface structure can be utilized to bind to agents either naturally (affinity to a receptor) or synthetically (functionalizing protein-protein interactions).

Platelets are the primary effectors of thrombosis in the human body. When the endothelium is injured, platelets play a key role in the repair process by first being activated, then adhering to the subendothelium. These steps lead to the aggregation of platelets that form the platelet plug. Platelet functions are inhibited by the endothelium in normal conditions, which limits unnecessary thrombosis from occurring. This process is called as endothelial thromboregulation (10, 11). During an injury, the endothelium regulates the platelet reactivity via complex signaling. However, in some pathological conditions, platelets functions can provide vital support to disease processes and could become essential to their basic pathophysiology (12). This is seen in several conditions including atherosclerotic vascular complications (13, 14), metastasis (15, 16), cancer associated venous thromboembolism (17), sepsis (18) and disseminated intravascular coagulation (19).

Platelet Decoy Surface Receptors and Activation:

The inventors have modified human platelets by a detergent procedure to obtain platelet decoys that are similar to platelets in their binding properties; however, they are incapable of activation or aggregation. Preliminary data shows that platelets become activated when stimulated with a wide range of agonists (i.e. Thrombin Receptor Activator Peptide (TRAP), adenosine diphosphate (ADP), collagen), while the platelet decoys do not (FIG. 2A). This was observed in spite of supraphysiological concentrations of agonists used (50 μM: TRAP, ADP or 5 μg/mL: collagen).

Some of the major platelet receptors are glycoprotein IIb-IIIa (GP IIb-IIIa, also known as integrin αIIbβ3), GP Ib-IX-V and GP VI. These receptors are involved in the processes of platelet activation or adhesion. GP IIb-IIIa is the main receptor with about 80,000 to 100,000 copies per platelet (20, 21) and displays extra recruitment from the intracellular pool in activated platelets (22). After stimuli (e.g. via agonists), this receptor undergoes a conformational change due to inside out signaling; as a result, GP IIb-IIIa can then bind fibrinogen or von Willebrand Factor (vWF) (23). The blockade of this conformational change inhibits platelet aggregation and as a consequence this receptor has been investigated extensively for antithrombotic therapy (2). GP IIb-IIIa and GP Ib-IX-V quantifications on platelet decoys have been evaluated and compared to parent platelets (FIGS. 2B and 2C). It has been shown that the presence of GP IIb and GP Ib was significantly reduced on the decoys compared to the platelet controls (FIGS. 2B and 2C). Indeed, decoys retain a degree of their GP IIb receptor binding function (FIG. 2B), but agonist-induced GP IIb recruitment at the decoy surface was not observed, unlike platelets (FIG. 2B). One can further quantify the changes in the surface receptor expression of decoys (compared to parent platelets) and evaluate if they are still able to bind their substrates (e.g. vWF (GP IIb-IIIa), fibrinogen (GP IIb-IIIa), collagen (GPVI)). Protease-Activated Receptors PAR-1 and PAR-4 are thrombin receptors, which once activated induce signaling that culminates in the morphological change of platelets and ADP release from dense granules (24). This release can further activate surrounding platelets due to ADP binding to the P2Y12 receptors on platelets. P2Y12 blockade constitutes a strategy used in clinics for anti-platelet therapies (3). A characterization of these relevant receptors, as well as GPVI (which binds to collagen(25)) can be performed by flow cytometry.

Platelet Decoy Aggregation and Anti-Platelet Effect:

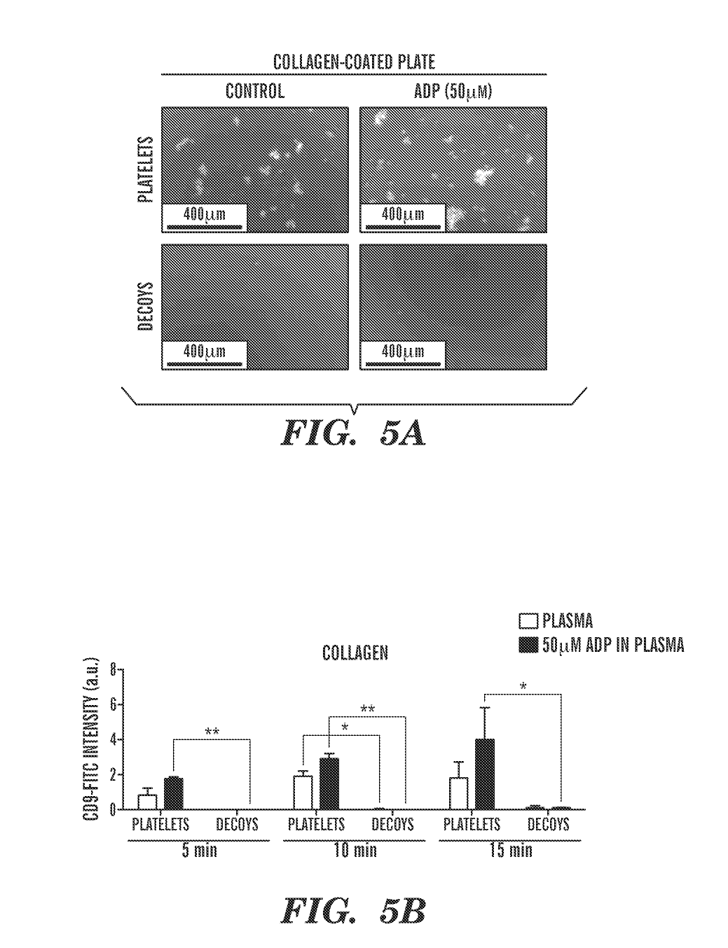

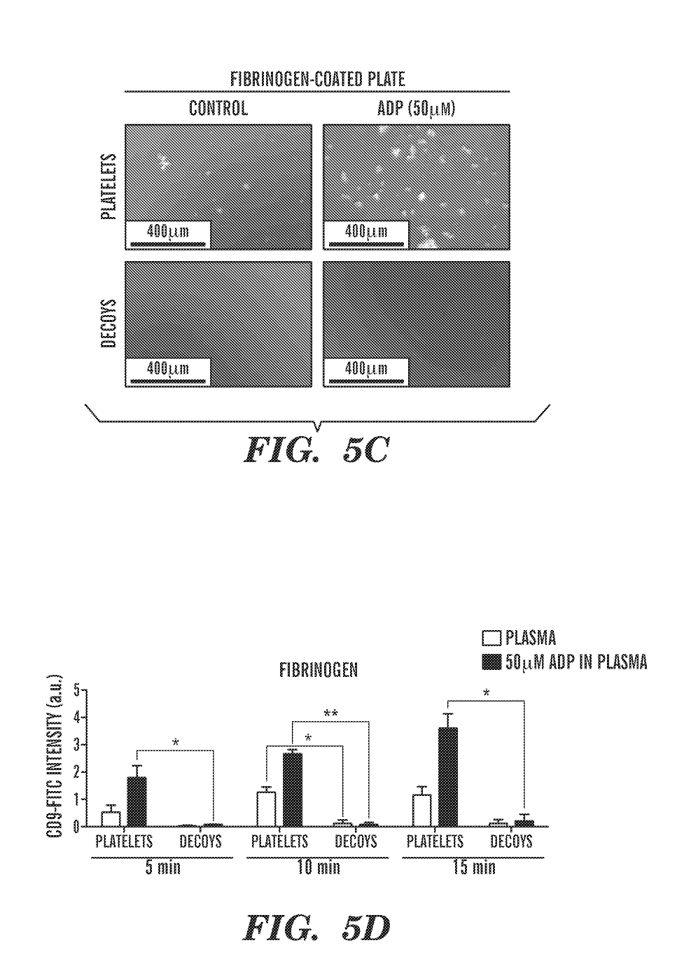

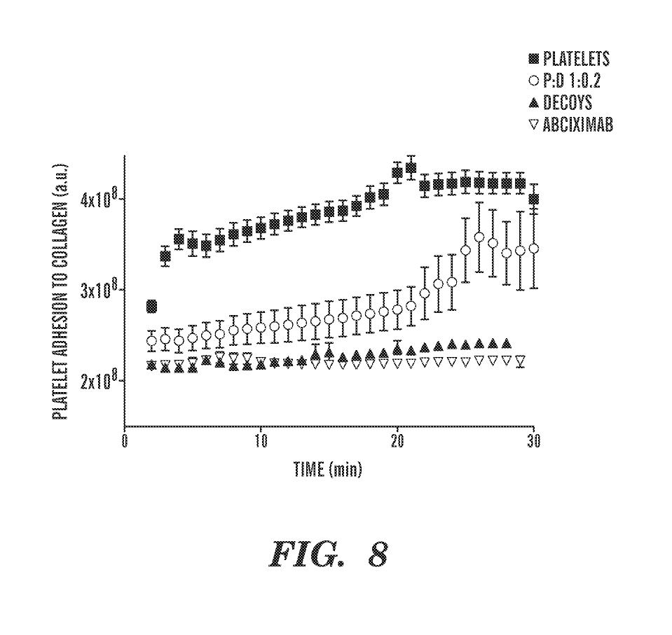

It is well established that the activation response of platelets is a precursor of their aggregation. The inventors compared the aggregation responses of platelets and decoys using light aggregometry. Platelet decoys were found to be incapable of aggregation with a high concentration of agonists, e.g. 50 μM ADP (FIG. 4A) or 10 μg/mL collagen (FIG. 4B), while their platelet counterparts show more than 50% aggregation under the same conditions. When decoys were co-incubated with platelets (P:D=1:0.25, 1:0.5, 1:1, 1:2), no obvious aggregation was observed over 20 min (FIG. 4C). Co-incubation of platelets and decoys with agonists highlighted that decoys decrease the magnitude of platelet aggregation (anti-platelet effect) (FIGS. 4D and 4E). In parallel, collagen and fibrinogen coated surfaces have been used as an in vitro injury model to test the reactivity of the decoys towards these surfaces over time and compared to normal platelets. Platelet aggregates were formed in a few minutes on both surfaces and increased over time (FIGS. 5A-5D). In addition, the effect was enhanced by concomitant incubation with an aggregation agonist (50 μM ADP) (FIGS. 5A-5D). However, decoys failed to aggregate on these surfaces both with prolonged exposure as well as ADP addition, corroborating the results obtained by aggregometry. These results altogether show that the platelet decoys are inert while facing chemical or contact stimuli normally leading to platelet activation and aggregation and thus might provide an anti-platelet effect. These results can be further validated by using collagen and fibrinogen-coated microfluidic chips (FIG. 8), to accurately represent in vivo behavior of blood flow. An exemplary microfluidic chip and assay for assaying platelet activation and aggregation is described in US Provisional Application No. PCT/US2014/060956, filed Oct. 16, 2014, content of which is incorporated herein by reference in its entirety. One can also used the BIOFLEX™ system from Fluxion Bioscienses for assessing platelet function. Additional microfluidic systems for assessing platelet function are described, for example, in Sarvepalli et al., Annals of Biomedical Engineering (2009), 27(7:1331-1341; Guttirrez et al., Lab Chip (2008), 8(9):1486-1495; Li et al., Lab Chip (2012), 12:1355-1362; and Colace et al., Annu Rev. Biomed. Eng. (2013) 15:283-303, the content of each of which is incorporated herein by reference in their entirety. The experiment can be processed under normal flow and with increased shear stress, which is known to impact platelet behavior. Further, the interaction of platelet decoys and endothelial cells can be investigated under high shear stress, using endothelial cell-coated microfluidic chips. Agonists can be used to highlight differences between platelets and decoys with regards to their behavior towards this artificial endothelial wall. TNF-α can be utilized to stimulate endothelial cells and study platelet decoy interactions with both endothelial cells and blood cells. Blockade of targeted receptors is a strategy to study various interaction mechanisms (with endothelial cells and also with blood cells and untreated platelets).

The life span of platelet decoys can be evaluated to further define their administration frequency in therapeutic scenarios. In literature, platelet life span has been studied by labeling platelets either in vitro or in vivo (e.g. with dye-conjugated antibodies) (26). It has been shown that apoptosis plays a significant role in determining the platelet life span (27). However, platelet life span does not increase indefinitely on inhibition of apoptosis but it is only extended by a few days (27). Apoptosis is consequently not the only factor involved in this process. Studies have shown that desialylation of glycosylated protein also induces the clearance of platelets in mice (28). Specific hepatocyte receptors have been identified that are able to detect these desialylated platelets and remove them from circulation (29). Without wishing to be bound by a theory, lack of apoptotic machinery in the decoys can lead to an increase in their life span. The life span of the functionally inert platelet decoys can be dependent on desialylation induced hepatic clearance.

The maximum tolerated dose by IV injection of platelet decoys can also be evaluated. The various injected boluses could represent 20, 33, 50 and 66% of the actual platelet count of the mouse. A histological evaluation of major organs (obtained by autopsy after the termination of the experiment) can be used for excluding any major complications observed with the decoys.

Approximately every 44 seconds, an American will have a myocardial infarction (6). Myocardial infarction along with unstable angina is a part of Acute Coronary Syndromes (ACS), which represented 625,000 patient discharges in 2010 (6). These disorders occur due to the obstruction of coronary arteries, often as a result of the formation of a platelet clot on the ruptured surface of an intravascular plaque (13). Patients with ACS may be candidates for an emergent Percutaneous Coronary Intervention (PCI) with a stent to enhance blood supply to the heart (myocardial revascularization) (35). The PCI procedure has revolutionized the management of patients with ACS and approximately 954,000 procedures were done in 2010 (6). During PCI intervention and following it, antiplatelet drugs (P2Y12 inhibitors) are administered to the patient to prevent periprocedural thrombotic complications and to reduce the incidence of clot formation in the stent (36, 37). Prasugrel (Effient®), ticagrelor (Brilinta®) and clopidogrel (Plavix®) are P2Y12 based platelet inhibitors, which are FDA approved in this setting. These drugs have the advantage of being administered orally, however, they are limited by their slow bioavailability, as they require gastrointestinal absorption. In addition, prasugrel and clopidogrel are prodrugs that require hepatic metabolism to become active. In patients with myocardial infraction, there can be a significant decline in the volume of blood being pumped by the heart and this leads to further reduction in absorption and metabolism. Thus it is apparent that in emergency situations requiring rapid and consistent platelet inhibition, the currently approved oral agents can fall short in many cases (38).

Platelet decoys disclosed herein can be used as an adjunctive antiplatelet therapy in the setting of a PCI. Indeed, platelet decoys disclosed herein have been shown to not self-aggregate under agonist stimuli (FIGS. 4A and 4B). Furthermore, they are able to mitigate platelet aggregation under agonist when decoys and platelets are co-incubated. This has been shown using both 5 μg/mL collagen (FIG. 4E) and 10 μM ADP (FIG. 4D) as agonists. Accordingly, the platelet decoys can be administered intravenously and they can provide a rapid onset of platelet inhibition. Currently approved drugs have a half-life between 6 to 9 hrs. If serious bleeding develops, therapeutic reversal requires platelet transfusions (39) that would not be fully effective due to the continued effect of the circulating drug on the newly transfused platelets (40). For instance, ex vivo experiments have shown that platelet aggregation of clopidogrel treated volunteers normalizes only after fresh platelets reach 90% of the total platelets (39). In contrast to the oral drugs, platelet transfusions would be immediately effective in reversing any serious bleeding side effects from decoys. In addition, anti-platelet drugs are often administered along with anti-coagulants or a second antiplatelet agent, though several serious metabolic interactions between these drugs are described (41).

The use of decoys allows the administration of an additional anti-platelet drug, with no change in the relevant metabolic pathways. As discussed above, a microfluidic chip-based injury model can be used to characterize differences in decoy and platelet aggregation on exposed collagen. This model can also be used as representative of a minor vascular injuries occurring during PCI. This model is used to determine the preventive effect of an increasing proportion of decoys in physiological blood flow conditions. For example, 20, 33 or 50% of decoys are added to platelet rich plasma, with and without the presence of agonists (such as TRAP, ADP, or collagen). If needed, an additional stimulus for activation can be provided by the chip by inducing high shear stress. The introduction of a foreign object into the vasculature, as in PCI, can induce vascular inflammation and promote clot formation. To simulate this pathway, a similar microfluidic chip based experiment can be conducted using TNF-alpha-stimulated endothelial cells. The combination of both these simulations can allow an investigation of the minimum proportions required for the desired preventive effect on clotting. Based on the preliminary results above decoys reduce or slow down platelet aggregation. In addition, one can evaluate the theoretical reversibility of this observed effect by the addition of extra platelets. The in vivo model to validate these results can be an arterial induced thrombosis model in mouse. Inventors have used this model in the past to assess the efficacy of clot busting particles in vivo using intravital microscopy (42). Decoys can be injected in any desired amount before in vivo endothelial injury induced by ferric chloride (topical application of a FeCl₃ saturated filter paper). This injury provokes a subsequent thrombotic response. The following criteria can be assessed by intravital microscopy: (i) the time required for the formation of a thrombus, (ii) the quantity of the platelets adhered to the injured endothelium, (iii) the rate of thrombus growth and (iv) the total time to complete vessel occlusion.

Another aspect of ACS where platelet decoys could play a pivotal role is as an adjunct to fibrinolytic therapy. Standard regimens of emergency fibrinolytic therapy in myocardial infarction and stroke consist of a combination of fibrinolytic tPA (tissue Plasminogen Activator) and anti-platelet therapy. Literature has shown that platelets potentiate the activation of inactive circulatory plasminogen by tPA (7, 43) and it has been hypothesized that the potentiating effect on fibrinolysis is due to the co-localization of tPA and plasminogen on the platelets' surface (8, 44).

Experiments with Texas red-labeled tPA showed a significant immobilization of the protein on Platelet Decoys (FIG. 6). A hypothetical contribution of endocytosis to this immobilization can be excluded, as this process is unchanged when the incubation is performed at 4° C. (FIG. 6). Interestingly, data shows that tPA was binding to the Decoys with about 80% binding efficiency (determined with a Texas red-tPA standard curve) and that tPA conserves it full activity compared to its free form (SensoLyte® AMC tPA Activity Assay Kit *Fluorimetric*, Anaspec, Calif.). Thus, tPA tagged platelet decoys can be used as a single injection fibrinolytic that can reduce hemorrhagic side effects. This allows focusing of tPA activity at the specific site of the clot.

Cancer metastasis is the biggest cause of mortality in cancer patients (45). Breast cancer patients often undergo curative surgery, which is followed by adjuvant therapy in patients at high risk of relapse (47). It has been recently reported that CTCs are detected in more than 90% of these non-metastatic breast cancer patients (48). However there are no currently available therapies that specifically act on CTCs in this setting. Targeting CTCs is particularly important, as they are the “seeds” for future metastases and the primary cause of recurrence in these patients. In the process of metastatic dissemination, tumor cells intravasate into the blood stream (forming CTCs) from the primary tumor site. These CTCs interact with platelets in the circulation and activate them, forming CTC-platelet aggregates. In response, platelets protect and assist the CTCs and also help in downstream end-organ extravasation (15, 25, 49). Experimental platelet depletion has also been shown to massively reduce metastasis (25). Here we hypothesize that platelet decoys would act as a potential drug free CTC targeting therapy in which they would interfere with the pro-oncogenic effects of normal platelets and disrupt the metastatic cascade, decreasing the incidence of metastasis. The decoys would compete for the surface of the cancer cells and would also reduce platelet aggregation by preventing magnification of the activation stimulus. Without wishing to be bound by a theory, decoys can prevent formation of large CTC-platelet aggregates and consequently make the CTCs more accessible for circulating immune cell identification and destruction. Additionally, the lack of big enough aggregates that can be entrapped in capillary microvessels could reduce the efficiency of metastasis (50). Data reported herein shows that platelet decoys are able to bind cancer cells, as normal platelets (FIGS. 3A-3H), despite the reduced amount of IIb/IIIa receptor present at their surface (FIG. 2B). Platelet IIb/IIIa receptors are known to be involved in this interaction (51, 52) and their blockade inhibits the interactions of both platelets and decoys with cancer cells (FIGS. 3I-3L). This approach is studied in an in vivo metastatic model. For example, the NOD/SCID mouse model is depleted of its endogenous platelets and used with human platelets. The experimental metastatic conditions are generated by the injection of a concentrated cancer cell bolus via the tail vein. After the termination of the experiments, differences in metastatic efficiency are quantified by estimating the total metastatic load in vital organs (obtained by autopsy).

The use of anti-platelet agents in the management of Acute Coronary Syndromes is well established. Decoys disclosed herein can be used in the management of these patients by providing immediate and consistent effects in an emergency setting. This is particularly true as an adjunct to Percutaneous Coronary Intervention (PCI) and fibrinolytic therapy. Their lack of drug interactions and their long half-life is convenient as the patient transitions to outpatient oral agents. Another application for platelet decoys is in adjuvant therapy of early breast cancer. Primary tumor does not kill cancer patients, however their metastases will ultimately be responsible for mortality in 90% of them (45). Decoy-based anti-platelet therapies can have a significant impact for some of these cancer patients.

### REFERENCES FOR EXAMPLE 2

1. Katz J N, Kolappa K P, Becker R C. Beyond thrombosis: the versatile platelet in critical illness. Chest. 2011; 139(3):658-68. Epub 2011 Mar. 3. doi: 10.1378/chest.10-1971. PubMed PMID: 21362652.
2. Armstrong P C, Peter K. GPIIb/IIIa inhibitors: from bench to bedside and back to bench again. Thromb Haemost. 2012; 107(5):808-14. Epub 2012 Mar. 1. doi: 10.1160/TH11-10-0727. PubMed PMID: 22370973.
3. Cattaneo M. New P2Y(12) inhibitors. Circulation. 2010; 121(1):171-9. Epub 2010/01/06. doi: 10.1161/CIRCULATIONAHA.109.853069. PubMed PMID: 20048234.
4. Angiolillo D J, Guzman L A, Bass T A. Current antiplatelet therapies: benefits and limitations. Am Heart J. 2008; 156(2 Suppl):53-9. Epub 2008 Jul. 31. doi: 10.1016/j.ahj.2008.06.003. PubMed PMID: 18657680.
5. Kolansky D M. Acute coronary syndromes: morbidity, mortality, and pharmacoeconomic burden. Am J Manag Care. 2009; 15(2 Suppl):536-41. Epub 2009 Apr. 16. PubMed PMID: 19355807.
6. Go A S, Mozaffarian D, Roger V L, Benjamin E J, Berry J D, Blaha M J, et al. Heart Disease and Stroke Statistics—2014 Update: A Report From the American Heart Association. Circulation. 2013. Epub 2013 Dec. 20. doi: 10.1161/01.cir.0000441139.02102.80. PubMed PMID: 24352519.
7. Sticker R B, Wong D, Shiu D T, Reyes P T, Shuman M A. Activation of plasminogen by tissue plasminogen activator on normal and thrombasthenic platelets: effects on surface proteins and platelet aggregation. Blood. 1986; 68(1):275-80. Epub 1986 Jul. 1. PubMed PMID: 2941084.
8. Miles L A, Plow E F. Binding and activation of plasminogen on the platelet surface. J Biol Chem. 1985; 260(7):4303-11. Epub 1985 Apr. 10. PubMed PMID: 3920216.
9. Cristofanilli M, Budd G T, Ellis M J, Stopeck A, Matera J, Miller M C, et al. Circulating tumor cells, disease progression, and survival in metastatic breast cancer. N Engl J Med. 2004; 351(8):781-91. Epub 2004 Aug. 20. doi: 10.1056/NEJMoa040766. PubMed PMID: 15317891.
10. Marcus A J, Safier L B. Thromboregulation: multicellular modulation of platelet reactivity in hemostasis and thrombosis. FASEB J. 1993; 7(6):516-22. Epub 1993/04/01. PubMed PMID: 8472890.
11. Marcus A, Broekman M, Drosopoulos J, Pinsky D, Islam N, Gayle R, 3rd, et al. Thromboregulation by endothelial cells: significance for occlusive vascular diseases. Arterioscler Thromb Vasc Biol. 2001; 21(2):178-82. Epub 2001 Feb. 7. PubMed PMID: 11156849.
12. Leslie M. Cell biology. Beyond clotting: the powers of platelets. Science. 2010; 328(5978):562-4. Epub 2010 May 1. doi: 10.1126/science.328.5978.562.
PubMed PMID: 20430990.
13. Davi G, Patrono C. Platelet activation and atherothrombosis. N Engl J Med. 2007; 357(24):2482-94. Epub 2007 Dec. 14. doi: 10.1056/NEJMra071014. PubMed PMID: 18077812.
14. Nieswandt B, Pleines I, Bender M. Platelet adhesion and activation mechanisms in arterial thrombosis and ischaemic stroke. J Thromb Haemost. 2011; 9 Suppl 1:92-104. Epub 2011 Aug. 4. doi: 10.1111/j.1538-7836.2011.04361.x. PubMed PMID: 21781245.
15. Gay L J, Felding-Habermann B. Contribution of platelets to tumour metastasis. Nat Rev Cancer. 2011; 11(2):123-34. Epub 2011 Jan. 25. doi: 10.1038/nrc3004. PubMed PMID: 21258396.
16. Erpenbeck L, Schon M P. Deadly allies: the fatal interplay between platelets and metastasizing cancer cells. Blood. 2010; 115(17):3427-36. Epub 2010 Mar. 3. doi: 10.1182/blood-2009-10-247296. PubMed PMID: 20194899; PubMed Central PMCID: PMC2867258.
17. Noble S, Pasi J. Epidemiology and pathophysiology of cancer-associated thrombosis. Br J Cancer. 2010; 102 Suppl 1:S2-9. Epub 2010 Apr. 23. doi: 10.1038/sj.bjc.6605599. PubMed PMID: 20386546; PubMed Central PMCID: PMC3315367.
18. Li Z, Yang F, Dunn S, Gross A K, Smyth S S. Platelets as immune mediators: their role in host defense responses and sepsis. Thromb Res. 2011; 127(3):184-8. Epub 2010 Nov. 16. doi: 10.1016/j.thromres.2010.10.010. PubMed PMID: 21075430; PubMed Central PMCID: PMC3042496.
19. Levi M, van der Poll T. Disseminated intravascular coagulation: a review for the internist. Intern Emerg Med. 2013; 8(1):23-32. Epub 2012 Sep. 28. doi: 10.1007/s11739-012-0859-9. PubMed PMID: 23015284.
20. Wagner C L, Mascelli M A, Neblock D S, Weisman H F, Coller B S, Jordan R E.
Analysis of GPIIb/IIIa receptor number by quantification of 7E3 binding to human platelets. Blood. 1996; 88(3):907-14. Epub 1996 Aug. 1. PubMed PMID: 8704248.
21. Niiya K, Hodson E, Bader R, Byers-Ward V, Koziol J A, Plow E F, et al. Increased surface expression of the membrane glycoprotein IIb/IIIa complex induced by platelet activation. Relationship to the binding of fibrinogen and platelet aggregation. Blood. 1987; 70(2):475-83. Epub 1987 Aug. 1. PubMed PMID: 3607284.
22. Addo J B, Bray P F, Grigoryev D, Faraday N, Goldschmidt-Clermont P J. Surface recruitment but not activation of integrin alpha IIb beta 3 (GPIIb-IIIa) requires a functional actin cytoskeleton. Arterioscler Thromb Vasc Biol. 1995; 15(9):1466-73. Epub 1995 Sep. 1. PubMed PMID: 7670962.
23. Savage B, Cattaneo M, Ruggeri Z M. Mechanisms of platelet aggregation. Curr Opin Hematol. 2001; 8(5):270-6. Epub 2001 Oct. 18. PubMed PMID: 11604561.
24. Covic L, Gresser A L, Kuliopulos A. Biphasic kinetics of activation and signaling for PAR1 and PAR4 thrombin receptors in platelets. Biochemistry. 2000; 39(18):5458-67. Epub 2000 May 23. PubMed PMID: 10820018.
25. Bambace N M, Holmes C E. The platelet contribution to cancer progression. J Thromb Haemost. 2011; 9(2):237-49. Epub 2010 Nov. 3. doi: 10.1111/j.1538-7836.2010.04131.x. PubMed PMID: 21040448.
26. Michelson A D, Barnard M R, Hechtman H B, MacGregor H, Connolly R J, Loscalzo J, et al. In vivo tracking of platelets: circulating degranulated platelets rapidly lose surface P-selectin but continue to circulate and function. Proc Natl Acad Sci USA. 1996; 93(21):11877-82. Epub 1996 Oct. 15. PubMed PMID: 8876231; PubMed Central PMCID: PMC38152.
27. Mason K D, Carpinelli M R, Fletcher J I, Collinge J E, Hilton A A, Ellis S, et al. Programmed a nuclear cell death delimits platelet life span. Cell. 2007; 128(6):1173-86. Epub 2007 Mar. 27. doi: 10.1016/j.cell.2007.01.037. PubMed PMID: 17382885.
28. Kotze H F, van Wyk V, Badenhorst P N, Heyns A D, Roodt J P, Lotter M G. Influence of platelet membrane sialic acid and platelet-associated IgG on ageing and sequestration of blood platelets in baboons. Thromb Haemost. 1993; 70(4):676-80. Epub 1993 Oct. 18. PubMed PMID: 8115995.
29. Rumjantseva V, Grewal P K, Wandall H H, Josefsson E C, Sorensen A L, Larson G, et al. Dual roles for hepatic lectin receptors in the clearance of chilled platelets. Nat Med. 2009; 15(11):1273-80. Epub 2009 Sep. 29. doi: 10.1038/nm.2030. PubMed PMID: 19783995.
30. Diehl K H, Hull R, Morton D, Pfister R, Rabemampianina Y, Smith D, et al. A good practice guide to the administration of substances and removal of blood, including routes and volumes. J Appl Toxicol. 2001; 21(1):15-23. Epub 2001 Feb. 17. PubMed PMID: 11180276.
31. Holbrook L, Moore C, Sanz-Rosa D, Solomon A, Emerson M. A NOD/SCID mouse model for the assessment of human platelet aggregation in vivo. J Thromb Haemost. 2012; 10(3):490-2. Epub 2011 Dec. 17. doi: 10.1111/j.1538-7836.2011.04595.x. PubMed PMID: 22171807.
32. Newman P J, Aster R, Boylan B. Human platelets circulating in mice: applications for interrogating platelet function and survival, the efficacy of antiplatelet therapeutics, and the molecular basis of platelet immunological disorders. J Thromb Haemost. 2007; 5 Suppl 1:305-9. Epub 2007 Aug. 1. doi: 10.1111/j.1538-7836.2007.02466.x. PubMed PMID: 17635741.
33. Pitchford S C, Yano H, Lever R, Riffo-Vasquez Y, Ciferri S, Rose M J, et al. Platelets are essential for leukocyte recruitment in allergic inflammation. J Allergy Clin Immunol. 2003; 112(1):109-18. Epub 2003 Jul. 9. PubMed PMID: 12847487.
34. Chauhan A K, Kisucka J, Lamb C B, Bergmeier W, Wagner D D. von Willebrand factor and factor VIII are independently required to form stable occlusive thrombi in injured veins. Blood. 2007; 109(6):2424-9. Epub 2006 Nov. 23. doi: 10.1182/blood-2006-06-028241. PubMed PMID: 17119108; PubMed Central PMCID: PMC1852205.
35. Deb S, Wijeysundera H C, Ko D T, Tsubota H, Hill S, Fremes S E. Coronary artery bypass graft surgery vs percutaneous interventions in coronary revascularization: a systematic review. JAMA. 2013; 310(19):2086-95. Epub 2013 Nov. 19. doi: 10.1001/jama.2013.281718. PubMed PMID: 24240936.
36. Steg P G, Harrington R A, Emanuelsson H, Katus H A, Mahaffey K W, Meier B, et al. Stent thrombosis with ticagrelor versus clopidogrel in patients with acute coronary syndromes: an analysis from the prospective, randomized PLATO trial. Circulation. 2013; 128(10):1055-65. Epub 2013 Aug. 1. doi: 10.1161/CIRCULATIONAHA.113.002589. PubMed PMID: 23900047.
37. Steinhubl S R, Berger P B, Mann J T, 3rd, Fry E T, DeLago A, Wilmer C, et al. Early and sustained dual oral antiplatelet therapy following percutaneous coronary intervention: a randomized controlled trial. JAMA. 2002; 288(19):2411-20. Epub 2002 Nov. 19. PubMed PMID: 12435254.
38. Taylor G, Osinski D, Thevenin A, Devys J M. Is platelet transfusion efficient to restore platelet reactivity in patients who are responders to aspirin and/or clopidogrel before emergency surgery? J Trauma Acute Care Surg. 2013; 74(5):1367-9. Epub 2013/04/24. doi: 10.1097/TA.0b013e31828cca61. PubMed PMID: 23609292.
39. Makris M, Van Veen J J, Tait C R, Mumford A D, Laffan M. Guideline on the management of bleeding in patients on antithrombotic agents. Br J Haematol. 2013; 160(1):35-46. Epub 2012 Nov. 3. doi: 10.1111/bjh.12107. PubMed PMID: 23116425.
40. Zafar M U, Santos-Gallego C, Vorchheimer D A, Viles-Gonzalez J F, Elmariah S, Giannarelli C, et al. Platelet function normalization after a prasugrel loading-dose: time-dependent effect of platelet supplementation. J Thromb Haemost. 2013; 11(1):100-6. Epub 2012 Nov. 10. doi: 10.1111/jth.12058. PubMed PMID: 23137352.
41. Siller-Matula J M, Trenk D, Krahenbuhl S, Michelson A D, Delle-Karth G. Clinical implications of drug-drug interactions with P2Y12 receptor inhibitors. J Thromb Haemost. 2014; 12(1):2-13. Epub 2014 Jan. 11. doi: 10.1111/jth.12445. PubMed PMID: 24406062.
42. Korin N, Kanapathipillai M, Matthews B D, Crescente M, Brill A, Mammoto T, et al. Shear-activated nanotherapeutics for drug targeting to obstructed blood vessels. Science. 2012; 337(6095):738-42. Epub 2012 Jul. 7. doi: 10.1126/science.1217815. PubMed PMID: 22767894.
43. Deguchi K, Murashima S, Shirakawa S, Soria C, Soria J, Dunn F, et al. The potentiating effect of platelet on plasminogen activation by tissue plasminogen activator. Thromb Res. 1985; 40(6):853-61. Epub 1985 Dec. 15. PubMed PMID: 3937289.
44. Michelson A D. Platelets, 3rd Edition. Elsevier. 2013.
45. Pantel K, Alix-Panabieres C. Circulating tumour cells in cancer patients: challenges and perspectives. Trends Mol Med. 2010; 16(9):398-406. Epub 2010 Jul. 30. doi: 10.1016/j.molmed.2010.07.001. PubMed PMID: 20667783.
46. Toriola A T, Colditz G A. Trends in breast cancer incidence and mortality in the United States: implications for prevention. Breast Cancer Res Treat. 2013; 138(3):665-73. Epub 2013 Apr. 3. doi: 10.1007/s10549-013-2500-7. PubMed PMID: 23546552.
47. Senkus E, Kyriakides S, Penault-Llorca F, Poortmans P, Thompson A, Zackrisson S, et al. Primary breast cancer: ESMO Clinical Practice Guidelines for diagnosis, treatment and follow-up. Ann Oncol. 2013; 24 Suppl 6:vi7-23. Epub 2013 Aug. 24. doi: 10.1093/annonc/mdt284. PubMed PMID: 23970019.
48. Fischer J C, Niederacher D, Topp S A, Honisch E, Schumacher S, Schmitz N, et al. Diagnostic leukapheresis enables reliable detection of circulating tumor cells of nonmetastatic cancer patients. Proc Natl Acad Sci USA. 2013; 110(41):16580-5. Epub 2013 Sep. 26. doi: 10.1073/pnas.1313594110. PubMed PMID: 24065821; PubMed Central PMCID: PMC3799344.
49. Labelle M, Begum S, Hynes R O. Direct signaling between platelets and cancer cells induces an epithelial-mesenchymal-like transition and promotes metastasis. Cancer Cell. 2011; 20(5):576-90. Epub 2011 Nov. 19. doi: 10.1016/j.ccr.2011.09.009. PubMed PMID: 22094253; PubMed Central PMCID: PMC3487108.
50. Lowe K L, Navarro-Nunez L, Watson S P. Platelet CLEC-2 and podoplanin in cancer metastasis. Thromb Res. 2012; 129 Suppl 1:S30-7. Epub 2012 Jun. 15. doi: 10.1016/S0049-3848(12)70013-0. PubMed PMID: 22682130.
51. Jurasz P, Alonso-Escolano D, Radomski M W. Platelet—cancer interactions: mechanisms and pharmacology of tumour cell-induced platelet aggregation. Br J Pharmacol. 2004; 143(7):819-26. Epub 2004 Oct. 20. doi: 10.1038/sj.bjp.0706013. PubMed PMID: 15492016; PubMed Central PMCID: PMC1575943.
52. Kitagawa H, Yamamoto N, Yamamoto K, Tanoue K, Kosaki G, Yamazaki H. Involvement of platelet membrane glycoprotein Ib and glycoprotein IIb/IIIa complex in thrombin-dependent and -independent platelet aggregations induced by tumor cells. Cancer Res. 1989; 49(3):537-41. Epub 1989 Feb. 1. PubMed PMID: 2910473.
53. Levi M, Eerenberg E, Kamphuisen P W. Bleeding risk and reversal strategies for old and new anticoagulants and antiplatelet agents. J Thromb Haemost. 2011; 9(9):1705-12. Epub 2011 Jul. 7. doi: 10.1111/j.1538-7836.2011.04432.x. PubMed PMID: 21729240.

### Example 3

### Inhibition of Metastases by Disrupting Platelet-Cancer Cell Interactions Using Ex Vivo Modified Platelets

Metastasis is the primary cause of mortality in breast cancer. At present there is no current availability of direct anti-metastatic therapy that can delay or interrupt this process and most importantly there is no cure for metastasis. There is an urgent need to develop therapeutics targeting this process, as it can be the pivotal step in concretely delaying the terminal stage of the disease. Thus, there is a need in the art for reducing, inhibiting or eliminating the mortality associated with metastatic breast cancer. In addition, there is also a need for reduction of therapy-associated toxicity. The platelet decoys disclosed herein can meet one or both of these goals.

Circulating tumor cells (CTCs) have been shown to rely on platelets to survive in the bloodstream, to escape from immune surveillance and to be actively anchored to the endothelium which is often followed by their extravasation and the formation of established metastases (2). The inventors have developed functionally inert platelets (called “platelet decoys”) from donor platelets that exert dominant negative antiplatelet effects (i.e. significantly decrease both platelet aggregation under agonists and arrest under flow) and that are able to bind breast cancer cells (3). Without wishing to be bound by a theory, the decoys can competitively bind to CTCs without being able to exert the functional pro-oncogenic effects of platelets. As a result, a significant reduction in the efficiency of the metastatic process can be expected. Further, in the absence of circulatory protection exerted by platelets, CTCs would be left vulnerable to immune identification and destruction, particularly by circulating natural killer (NK) cells.

Without wishing to be bound by a theory, decoys can directly hinder platelet-CTC interaction and reduce the efficiency of each step of the metastatic cascade. Thus, it can be important to demonstrate the ability of platelet decoys to inhibit cancer cell-platelet binding and aggregation as well as adhesion to endothelial cells in vitro and to evaluate the difference in NK cell-induced cancer cell destruction in vitro when cancer cells are pre-incubated with platelets or decoys or a mixture of both.

Inhibition of Tumor Cell-Induced Platelet Aggregation (TCIPA) is possible using antiplatelet drugs (20) and has been shown to decrease metastasis (21). As discussed herein, agonists are not able to activate and aggregate decoys. The inventors have shown decoys decrease platelet aggregation (FIGS. 4A-4E). Thus, without wishing to be bound by a theory, TCIPA could be reduced in presence of decoys.

Light aggregometry is used to assess whether Tumor Cell Induced Platelet Aggregation is decreased in presence of platelet decoys (in line with the observation that decoys reduce platelet aggregation by agonists). Flow cytometry studies can also be used for confirmation, using a marker of platelet activation (PAC-1), e.g., a dye conjugated PAC-1 antibody. Microfluidic studies (first using collagen then endothelial cell coated chips) helps one to assess, in conditions similar to vascular blood flow, the platelet to decoy ratio at which decoys maximize their antiplatelet effect including the prevention of active arrest of flowing tumor cells. Exemplary controls for this study are cancer cells flowing in (1) platelet-depleted blood, (2) platelet-depleted blood with decoys, and (3) blood. PAC-1 binds to GPIIb/IIIa only on activated platelets and thus allows one to quantify activation levels in various groups.

In another example, a microfluidic chip coated with collagen, such as described in Example 4, is used to study the inability of platelet decoys to bind collagen as well as their ability to decrease platelet adhesion to exposed collagen under flow. This is also used to find the ideal platelet to decoy ratio (P:D ratio) at which decoys can exert their maximal antiplatelet effect.

Once the platelets interact with cancer cells, the antiplatelet effect of decoys allows for a reduction in additional recruitment of platelets on their surface and in preventing platelet-fibrin-cancer cell aggregates. This can be helpful in increasing CTC surface accessibility for NK cells. To assess whether NK cells are able to induce cancer cell apoptosis when cancer cells are pre-incubated with (1) platelets only, (2) decoys only, and (3) a mixture of both. Cell-mediated cytotoxicity and apoptosis assays are used for this evaluation.

To study the effect of pre-incubation of breast cancer (MDA-MB-231, MCF-7) cells with platelets or decoys or a mixture of both before contact with NK cells is assessed in vitro using 7-AAD/CFSE cell-mediated cytotoxicity assay as well as apoptosis assay (Annexin V, PI). Without wishing to be bound by a theory, the anti-platelet effect induced by platelet can enable NK cells to access and destroy breast cancer cells since the anti-platelet effect can significantly decrease fibrin associated platelet thrombi around tumor cells.

It can also be essential to determine platelet decoy life span and its effects on blood coagulation. In one example, the life span of platelet decoys is determined in a NOD/SCID mouse. Flow cytometry is used to follow decoys' fate and compare it to human platelets. Human and mice platelets are distinguished by using species-specific antibodies and also their substantially different sizes can easily enable population gating. The effect of decoys on coagulation, i.e., if the decoys interfere with coagulation, is also evaluated by measuring bleeding times after their injection in mice. For example, human platelet decoys or platelets are injected in a NOD/SCID mouse model and their in vivo fate is monitored. This immunosuppressed model can be essential in studying human platelets in mice as human cells will not be identified and cleared.

The use of human platelets in NOD/SCID model has been successfully explored to characterize the life span and functions of human platelets in mice (22-24). This model is used to study the human platelet decoys in a mouse model. A translational gap exists in studying mouse platelets as they significantly differ from human platelets: e.g., thrombin acts via PAR3 in mice and PAR1 in humans (25) and they also have different platelet counts (26). Also, decoy production from mice can be cumbersome, as it can require sacrificing a large number of mice to achieve workable blood volume. Platelets or platelet decoys are injected in NOD/SCID mice and blood drawn every alternate day to evaluate decoy life span. For example, (i) whole blood cell count is assessed using a HEMAvet instrument, (ii) flow cytometric count post-labeling of human platelets, decoys and mice platelets is determined, by comparing the collected events to a known concentration of fluorescent beads added to each sample. The size of mice and human platelets is different and the two populations are delineated based on size and granularity. Additional controls using specific mice or human labeled antibodies are used to distinguish the two populations. Based on initial life span data, the dose and frequency of administration of decoys towards achieving consistent levels in the blood for future experiments are defined. After determining the decoys versus platelet count over time, (iii) effect of platelet decoys on normal coagulation cascade in vivo is evaluated. This is done by measuring bleeding time.

Without wishing to be bound by a theory, based on preliminary data, decoys can bind to cancer cells but do not actively arrest them in the vasculature. As such, they can fail to provide CTCs with pro-oncogenic factors, as decoys are inert and they can shield CTCs from platelet support and immune system identification. The decreased support towards CTCs can reduce metastatic dissemination in vivo.

In one example, fluorescent breast cancer cells (e.g., GFP labeled MDA-MB-231 cells) are incubated with decoys or platelets or a mixture of both. They are then injected as an intravenous bolus. After a period of growth, the metastatic load in major organs and compare among groups is enumerated. For example, the metastatic load in lung, liver and heart and also the blood CTC levels is determined after 2 weeks. Frequency of decoy administration and dose can be optimized to maximize the anti-metastatic effect.

In one example, an in vivo metastatic model is used. Prior evidence has highlighted that platelets make cancer cells more capable of metastasis (5, 9). We would assess if decoys have any inhibitory effects on the in-vivo efficacy of the metastatic process and tumor growth. GFP-labeled MDA-MB-231 breast cancer cells (with retroviral transduction (27)) are incubated with human platelets or decoys or a mixture before being intravenously injected to mice. Various organs, such as lungs, liver, spleen and heart, are collected two weeks after injection of GFP labeled tumor cells. The metastatic load in these organs is measured by fluorescence detection using a live animal imaging system (IVIS). In addition, blood samples are collected and CTC detection by flow cytometry is performed. Metastatic load is also measured after harvesting, homogenizing and dissolving the organs followed by measurement of GFP fluorescence level in comparison to a standard of solubilized GFP MDA-MB-231 cells. Alternatively, mice are depleted of their endogenous platelets and provided with human platelets (23) before injecting a tumor cell bolus via the tail vein.

The method disclosed herein provides patients with a drug free therapy based on cutting off vital platelet support to circulating cancer cells. This can lead to a profound impact in breast cancer management where metastases could be potentially delayed or CTCs destroyed before they become threatening. The cell based method and the lack of a drug indicate that the side effects are expected to be low or nonexistent. Further, as described herein, platelets decoys could also be made from the patient's own platelets, decreasing the abnormal elevated platelet count seen in many cancer patients that has been associated with poor prognosis. Based on the inventors' in vitro studies, platelet decoys can exert efficiency with only one or two platelet transfusion(s). This is a paradigm shift in cancer management and accelerates progress toward combating breast cancer.

The major cause of mortality in cancer patients ultimately comes down to metastatic dissemination to vital organs. To date, treatments and drugs have been developed and approved to treat localized and distant tumors. However prevention and efforts to limit tumor spread is a perpetual research challenge and importantly there is no cure for metastases. Hence there is an urgent need for new strategies to combat this last stage of the disease where the 5-year relative survival of patients drops down from 72% (stage III) to 22% (stage IV) (according to the National Cancer Institute database, 2014). Thus, one of the aims of this study is to develop novel therapeutic approaches to delay or prevent the development of stage IV breast cancer.

Platelets play a central role in the metastatic cascade. This fact is illustrated by past findings that showed that the metastatic dissemination is significantly reduced in mice lacking platelets (4), and that an increase in platelet count, which is seen in 10 to 57% of cancer patients, is associated with poor prognosis (5). Circulating tumor cells (CTCs) enter the bloodstream by intravasation from the primary tumor site to the vasculature where they are then free to disseminate to distant organs. However, the bloodstream represents a harsh environment for these cells, with blood flow, shear stress and immune cells being major contributors to tumor cell destruction. However, platelets promote metastasis by supporting the survival and engraftment of CTCs in this harsh environment. First, platelets bind to CTCs and this interaction generates signaling loops between the platelets and CTCs that induce platelet activation and aggregation. At the same time, platelets release pro-oncogenic and angiogenic factors that are able to support the survival of CTCs in the bloodstream and at distant sites. Platelets also provide CTCs with protection against immune cells by coating the cancer cell surface and physically shielding them. In addition, activated platelets facilitate cancer cell arrest within the vasculature by forming large cell aggregates that are more easily captured in small microvessels. In this manner, platelet support during the varied steps of the metastatic process increases the likelihood that CTCs will survive in the bloodstream and form metastases in distant organs. There is no current approach in place in the clinic (i.e. no approved therapy) to prevent and limit metastasis, and platelet-CTC interactions have not been targeted for anti-metastatic therapy in the past.

The approach disclosed in the present study is based on inventors′development of methods to extract living human (or other mammalian) platelets with detergents to create inert “platelet decoys” that compete with normal platelets for binding to CTCs, and suppress subsequent platelet activation and aggregation cascades because they are unable to activate themselves. Thus, without wishing to be bound by a theory, by acting in a dominant negative manner to decrease the normal platelets' accessibility to CTCs, the platelet decoys can inhibit their ability to survive and arrest within the vasculature, and thus they could represent an new cell-based therapeutic for preventing breast cancer metastasis.

These platelet decoys represent a paradigm shift in current anti-cancer therapies because they specifically target cancer dissemination, which is the major cause of death in cancer patients. They also can induce lower side effects than conventional anti-cancer therapies because they are not generally cytotoxic. Even if toxicity were observed, the treatment could be reversed rapidly in a controlled manner by infusing normal (intact/functional) platelets. Finally, platelet decoys can have a dual therapeutic effect because they also inhibit platelet-platelet interactions, and thus, they can decrease the incidence of cancer-associated thrombosis, which can lead to acute life-threatening events in patients with cancer. Thus, the present study describes a new cellular therapy that can significantly decrease mortality in cancer patients by preventing metastatic spread without producing systemic toxicities.

Metastasis is the leading cause of cancer patient mortality (6). When CTCs extravasate in the bloodstream where a very small fraction of them survive (7), leukocytes and platelets are caretakers that facilitate their continued survival and dissemination (8). Direct platelet and cancer cell contact induces signaling that promotes EMT (epithelial-mesenchymal transition), enhancing their invasiveness and formation of new metastasis (9).

Platelet inhibition or depletion has also been shown to decrease metastasis in vivo (5). In addition, they play a determinant role in angiogenesis (10) and their inhibition (using antiplatelet drugs) increases the response to chemotherapeutic drugs in vivo (11, 12). Hence, platelets support cancer at multiple levels during dissemination, angiogenesis and therapy. They also protect tumor cells against immune cell detection. Particularly, the shielding layer formed by platelet accumulation around CTCs has been shown to block natural killer (NK) cell identification (13, 14). They are consequently excellent targets to prevent and cut off the fatal metastatic stage of the disease.

At present, no therapies are available that directly act on the dissemination of breast cancer cells in the circulation. There is ongoing interest in exploring a potential role of aspirin in reducing metastasis. However, there is conflicting evidence about its effects and according to the National Cancer Institute (in April 2014), there is no definite clarity on whether aspirin can prevent cancer and its dissemination. Currently, clinical trials are ongoing in order to collect more data. Thus, there is an unmet need to urgently develop novel strategies to reduce the efficiency of metastatic dissemination.

The present study describes disrupting support to CTCs using platelet decoys. Without wishing to be bound by a theory, the platelet decoys can work in multiple ways: (a) inducing an antiplatelet effect to avoid aggregation and arrest at the endothelium, (b) physically shielding platelet-CTC interaction to decrease/prevent signaling and pro-oncogenic factor supply, (c) preventing/reducing direct contact between platelets and CTCs, shown to be responsible for enhanced invasiveness.

Platelet decoys can directly disrupt platelet-CTC interaction while platelets can still execute basal hemostatic repair function. This strategy is based on absence of drugs and is expected not to induce severe side effects commonly associated with cytotoxic drugs.

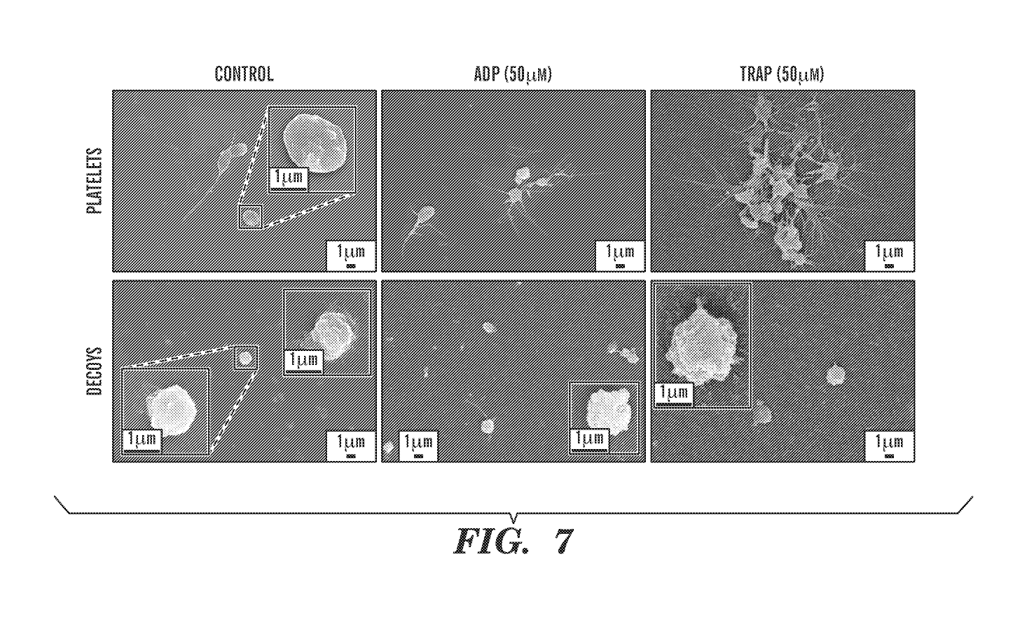

As described in the present disclosure, the inventors have developed platelet decoys that are fundamentally inert platelets. These detergent treated platelets from healthy donors retain some degree of platelet surface receptor expression (¼^{th} GPIIb/IIIa and ⅓^{th} GP Ib/IX/V remaining) (FIGS. 2B and 2C), have the morphological appearance of resting platelets (FIG. 7) but fail to activate (FIG. 2A) and aggregate (FIGS. 4A-4E) even with a supra-physiological dose of agonists (TRAP, ADP, collagen) compared to normal platelets. In addition, SEM (Scanning Electron Microscopy) observations of agonist incubated-platelets versus decoys have shown that decoys do not change shape while platelets undergo “egg shape” like spread and filopodia production that characterize their activation (FIG. 7). The function of these platelet decoys is to mimic platelets in order to interact with cancer cells but not respond like platelets. Platelets release factors and cytokines during activation (5), promote CTC arrest at the endothelium (15), create thrombi by activation and aggregation at the CTC surface (16) and protect CTCs from immune cells (13, 14). As decoys are functionally inert, they would be incapable of providing assistance to CTCs and would reduce direct contact between platelets and CTCs, which has been shown to be essential during metastasis (9). Consequently platelet decoys would greatly impair the vital platelet support to CTCs in circulation.

When co-incubated with platelets, platelet decoys also have the ability to decrease the aggregation response of platelets to agonists (antiplatelet effect) (FIGS. 4D and 4E) and importantly they do not adhere to thrombogenic surfaces (i.e. collagen) under flow conditions in microfluidic channels (that mimic the blood flow in vessels) (FIG. 8). They also decrease the ability of platelets to adhere to collagen (P:D ratio correspond to platelet to decoy ratio, FIG. 8). Platelet aggregation and adhesion are keys parameters in supporting CTCs. Thus, without wishing to be bound by a theory, the decoys can hamper metastasis.

Preliminary data indicate that platelet decoys interact with breast cancer cells (MDA-MB-231 and MCF-7) as much as platelets do. In one example, cancer cells were stained (with Hoechst) before being incubated with platelets or decoys (stained with PE-conjugated CD-9 antibody) in plasma and the interaction was subsequently assessed by flow cytometry (FIGS. 3C-3H). Platelet GPIIb/IIIa receptors are known to mediate the cancer cell-platelet interaction (5) and their blockade inhibits their binding (FIG. 3I-3L). Importantly, the lower expression of GPIIb/IIIa on the surface of decoys (compared to platelets, FIG. 2B) is not detrimental to their interaction with cancer cells.

Fluorescent cancer cells were perfused in a collagen-coated microfluidic device with blood versus decoy-supplemented blood in order to evaluate any changes in their arrest on a thrombogenic surface. Collagen represents a simple and effective model to study the arrest of tumor cells as the two main platelet receptors for collagen, GPIb/IX/V and GPVI, have been shown to play a pivotal role during metastasis in mice models (17, 18). Mice lacking these receptors exhibit significantly less metastases than the wild type control. Thus, the inventors selected this model as a starting point for the above-noted reasons. The effect of decoys on breast cancer cell arrest can also be studied using endothelial cell-coated microfluidic devices. Specifically, samples were perfused through a collagen-coated microfluidic device at a flow rate of 1,200 μL/hour that corresponds to 6.25 dyne/cm² and the platelet adhesion on collagen ROIs (regions of interest) was recorded in real time, with a fluorescence microscope that focuses on the APC signal from platelets.

Results showed that platelet decoy supplemented blood had significantly decreased platelet adhesion on the collagen ROIs in comparison to the control blood sample (FIG. 8). In the platelet-depleted blood with added decoys, decoys do not adhere to the surface at all (FIG. 8). Platelets did not adhere to the collagen ROIs as well in the negative control (blood incubated with the platelet inhibitor Abciximab) (FIG. 8). Data were also visualized on movies focusing on a single ROI (data not shown).

The overall usefulness of platelet decoys can be dependent on efficient modification of fragile platelets. The methods disclosed in the present invention for manipulation of platelets to form decoys leads to high yields. Preliminary data indicate a current yield of 60%. In the context of the therapy and because many cancer patients have an abnormal high platelet count (10 to 57% of cancer patients according to [1]), it is feasible to collect platelets from the patients themselves to produce decoys, and in parallel lower their platelet counts to normal.

The work reported herein demonstrates that platelet decoys are able to interact with platelets and cancer cells but they cannot be activated or induced to aggregate. Additionally, they induce an antiplatelet effect. Thus, without wishing to be bound by a theory, platelet decoys can prevent metastatic spread supported by tumor cell-platelet aggregation as well as life-threatening thrombotic events induced by platelet-platelet interactions in cancer patients. The data described herein also demonstrate that platelet decoys can competitively interact with cancer cells in the presence of platelets in flowing blood in vitro.

The work by the inventors have shown that the platelet decoys have similar binding to both human MDA-MB-231 and MCF-7 breast cancer cells when compared with intact platelets (FIGS. 3C-3H), despite the reduced amount of the receptor responsible for their interaction at their surface (FIG. 2B). Thus, without wishing to be bound by a theory, platelet decoys can prevent formation of microthrombi made of tumor cells, platelets and fibrin by interfering with the platelet-platelet interaction that supports platelet thrombi growth around tumor cells. Additionally, CTC coated by the modified platelets will not create big enough aggregates to induce embolism and capture in capillary microvessels, as compared to activated platelet-CTC complexes where additional platelets and fibrin are actively recruited to form large emboli (19). Finally and importantly, platelet decoys do not arrest on exposed collagen at the endothelium and hence may not promote extravasation.

As discussed in elsewhere in the disclosure, the platelet life span is regulated by apoptosis (balance between Bcl-xL and Bak). For example, Bak−/− platelets have been shown to have an extended life span (28). Platelet Decoys could be identified in the spleen and the liver as “aged” platelets and be destroyed. This potential problem is addressed by (i) adjusting the dose used, (ii) using an alternative procedure to obtain decoys which retain some degree of their activation potential, and (iii) using, in one example, transfected platelets (with nucleic acid based, e.g., siRNA, antisense, and miRNA, based protein repression) to knockdown Bak expression before making them “platelet decoys”. It is noted that platelet transfection has been investigated in the art (29).

A potential problem can occur when using tail vein injections for the in vivo metastatic dissemination experiment. This model is cost and time effective, however, the injected cancer cells could also be entrapped in the lung vasculature only a few minutes after the injection (30) and this could alter the clarity of the results. Thus, an alternative is to use MDA-MB-231 directly injected in the mammary fat pad as they are documented to be spontaneously metastatic in NOD/SCID/γc^{null} (NOG) mice (31). Another alternative is to use a commercially available spontaneously metastatic mouse model.

Primary tumors are usually not directly responsible for the mortality of cancer patients, however their metastases will ultimately kill 90% of them (32). Decoy-based antiplatelet therapies disclosed herein can have a significant impact for cancer patients. It has been recently reported that CTCs are detected in more than 90% of non-metastatic breast cancer patients (33). This offers the possibility to administer the decoy-based antiplatelet therapy disclosed herein to this specific population of patients just after their surgery (adjuvant therapy). With current advances in screening mammography, patient with early breast cancer are being detected much more commonly and the therapy disclosed herein can fulfill their unmet needs.

The therapy disclosed herein can be administered once a week given the life span of platelets in the blood stream (5 to 9 days). Further, if the decoys are prepared from the patient's own platelets, this can reduce their abnormally high cancer induced platelet counts. However, the patient could also receive decoys prepared from healthy donors. The source of platelets is not limited and the therapy can be adapted to each patient's clinical situation. This represents a drug free therapy that can easily be dose titrated or reversed in case of any side effects since patients can receive many platelet transfusions. Thus, the platelet decoys represent a big step towards controlling and preventing metastases by making breast cancer cells susceptible to the human body's inherent defenses.

### REFERENCES FOR EXAMPLE 3

1 Changing the landscape for people living with metastatic breast cancer. Metastatic breast cancer analysis: executive summary, October 2014. MBCalliance.
2 L. J. Gay and B. Felding-Habermann, 2011, Contribution of platelets to tumour metastasis, Nat Rev Cancer, 11, 2, 123-34.
3 .
4 E. Camerer, A. A. Qazi, D. N. Duong, I. Cornelissen, R. Advincula and S. R. Coughlin, 2004, Platelets, protease-activated receptors, and fibrinogen in hematogenous metastasis, Blood, 104, 2, 397-401,
5 N. M. Bambace and C. E. Holmes, 2011, The platelet contribution to cancer progression, J Thromb Haemost, 9, 2, 237-49.
6 B. Weigelt, J. L. Peterse and L. J. van't Veer, 2005, Breast cancer metastasis: markers and models, Nat Rev Cancer, 5, 8, 591-602.
7 P. Mehlen and A. Puisieux, 2006, Metastasis: a question of life or death, Nat Rev Cancer, 6, 6, 449-58.
8 N. Reymond, B. B. d'Agua and A. J. Ridley, 2013, Crossing the endothelial barrier during metastasis, Nat Rev Cancer, 13, 12, 858-70.
9 M. Labelle, S. Begum and R. O. Hynes, 2011, Direct signaling between platelets and cancer cells induces an epithelial-mesenchymal-like transition and promotes metastasis, Cancer Cell, 20, 5, 576-90.
10 J. Kisucka, C. E. Butterfield, D. G. Duda, S. C. Eichenberger, S. Saffaripour, J. Ware, Z. M. Ruggeri, R. K. Jain, J. Folkman and D. D. Wagner, 2006, Platelets and platelet adhesion support angiogenesis while preventing excessive hemorrhage, Proc Natl Acad Sci USA, 103, 4, 855-60.
11 S. Sarangi, A. Pandey, A. L. Papa, P. Sengupta, J. Kopparam, U. Dadwal, S. Basu and S. Sengupta, 2013, P2Y12 receptor inhibition augments cytotoxic effects of cisplatin in breast cancer, Med Oncol, 30, 2, 567.
12 A. Pandey, S. Sarangi, K. Chien, P. Sengupta, A. L. Papa, S. Basu and S. Sengupta, 2014, Anti-platelet agents augment cisplatin nanoparticle cytotoxicity by enhancing tumor vasculature permeability and drug delivery, Nanotechnology, 25, 44, 445101.
13 B. Nieswandt, M. Hafner, B. Echtenacher and D. N. Mannel, 1999, Lysis of tumor cells by natural killer cells in mice is impeded by platelets, Cancer Res, 59, 6, 1295-300.
14 T. Placke, H. G. Kopp and H. R. Salih, 2012, The wolf in sheep's clothing: Platelet-derived “pseudo self” impairs cancer cell “missing self” recognition by NK cells, Oncoimmunology, 1, 4, 557-559.
15 J. H. Im, W. Fu, H. Wang, S. K. Bhatia, D. A. Hammer, M. A. Kowalska and R. J. Muschel, 2004, Coagulation facilitates tumor cell spreading in the pulmonary vasculature during early metastatic colony formation, Cancer Res, 64, 23, 8613-9.
16 J. S. Palumbo, K. E. Talmage, J. V. Massari, C. M. La Jeunesse, M. J. Flick, K. W. Kombrinck, M. Jirouskova and J. L. Degen, 2005, Platelets and fibrin(ogen) increase metastatic potential by impeding natural killer cell-mediated elimination of tumor cells, Blood, 105, 1, 178-85.
17 S. Jain, M. Zuka, J. Liu, S. Russell, J. Dent, J. A. Guerrero, J. Forsyth, B. Maruszak, T. K. Gartner, B. Felding-Habermann and J. Ware, 2007, Platelet glycoprotein Ib alpha supports experimental lung metastasis, Proc Natl Acad Sci USA, 104, 21, 9024-8.
18 S. Jain, S. Russell and J. Ware, 2009, Platelet glycoprotein VI facilitates experimental lung metastasis in syngenic mouse models, J Thromb Haemost, 7, 10, 1713-7.
19 K. L. Lowe, L. Navarro-Nunez and S. P. Watson, 2012, Platelet CLEC-2 and podoplanin in cancer metastasis, Thromb Res, 129 Suppl 1, S30-7.
20 C. Medina, S. Harmon, I. Inkielewicz, M. J. Santos-Martinez, M. Jones, P. Cantwell, D. Bazou, M. Ledwidge, M. W. Radomski and J. F. Gilmer, 2012, Differential inhibition of tumour cell-induced platelet aggregation by the nicotinate aspirin prodrug (ST0702) and aspirin, Br J Pharmacol, 166, 3, 938-49.
21 L. Erpenbeck and M. P. Schon, 2010, Deadly allies: the fatal interplay between platelets and metastasizing cancer cells, Blood, 115, 17, 3427-36.
22 D. W. Bougie, D. Nayak, B. Boylan, P. J. Newman and R. H. Aster, 2010, Drug-dependent clearance of human platelets in the NOD/scid mouse by antibodies from patients with drug-induced immune thrombocytopenia, Blood, 116, 16, 3033-8.
23 L. Holbrook, C. Moore, D. Sanz-Rosa, A. Solomon and M. Emerson, 2012, A NOD/SCID mouse model for the assessment of human platelet aggregation in vivo, J Thromb Haemost, 10, 3, 490-2.
24 P. J. Newman, R. Aster and B. Boylan, 2007, Human platelets circulating in mice: applications for interrogating platelet function and survival, the efficacy of antiplatelet therapeutics, and the molecular basis of platelet immunological disorders, J Thromb Haemost, 5 Suppl 1, 305-9.
25 H. Ishihara, A. J. Connolly, D. Zeng, M. L. Kahn, Y. W. Zheng, C. Timmons, T. Tram and S. R. Coughlin, 1997, Protease-activated receptor 3 is a second thrombin receptor in humans, Nature, 386, 6624, 502-6.
26 I. Ahrens and K. Peter, 2008, Humanizing mouse thrombi, Nat Biotechnol, 26, 1, 62-3.
27 A. Mammoto, K. M. Connor, T. Mammoto, C. W. Yung, D. Huh, C. M. Aderman, G. Mostoslaysky, L. E. Smith and D. E. Ingber, 2009, A mechanosensitive transcriptional mechanism that controls angiogenesis, Nature, 457, 7233, 1103-8.
28 E. C. Josefsson, C. James, K. J. Henley, M. A. Debrincat, K. L. Rogers, M. R. Dowling, M. J. White, E. A. Kruse, R. M. Lane, S. Ellis, P. Nurden, K. D. Mason, L. A. O'Reilly, A. W. Roberts, D. Metcalf, D. C. Huang and B. T. Kile, 2011, Megakaryocytes possess a functional intrinsic apoptosis pathway that must be restrained to survive and produce platelets, J Exp Med, 208, 10, 2017-31.
29 W. Hong, A. A. Kondkar, S. Nagalla, W. Bergmeier, Y. Jin, J. H. Herman and P. F. Bray, 2011, Transfection of human platelets with short interfering RNA, Clin Transl Sci, 4, 3, 180-2.
30 A. D. Michelson, 2013, Platelets, 3rd Edition, Elsevier,
31 M. Z. Dewan, H. Terunuma, S. Ahmed, K. Ohba, M. Takada, Y. Tanaka, M. Toi and N. Yamamoto, 2005, Natural killer cells in breast cancer cell growth and metastasis in SCID mice, Biomed Pharmacother, 59 Suppl 2, S375-9.
32 K. Pantel and C. Alix-Panabieres, 2010, Circulating tumour cells in cancer patients: challenges and perspectives, Trends Mol Med, 16, 9, 398-406.
33 J. C. Fischer, D. Niederacher, S. A. Topp, E. Honisch, S. Schumacher, N. Schmitz, L. Zacarias Fohrding, C. Vay, I. Hoffmann, N. S. Kasprowicz, P. G. Hepp, S. Mohrmann, U. Nitz, A. Stresemann, T. Krahn, T. Henze, E. Griebsch, K. Raba, J. M. Rox, F. Wenzel, C. Sproll, W. Janni, T. Fehm, C. A. Klein, W. T. Knoefel and N. H. Stoecklein, 2013, Diagnostic leukapheresis enables reliable detection of circulating tumor cells of nonmetastatic cancer patients, Proc Natl Acad Sci USA, 110, 41, 16580-5.

All patents and other pSEublications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

1.-43. (canceled)

44. A modified platelet cell, wherein the modified platelet is substantially free of at least one membrane lipid or molecular component present in an unmodified platelet cell, and wherein the modified platelet cell is capable of binding to a cell ligand or platelet receptor substrate but does not promote cell aggregation and/or stimulate blood coagulation.

45. The modified platelet cell of claim 44, wherein said modified platelet cell retains at least one receptor selected from the group consisting of any of the following receptors: glycoprotein (GP) IIb/IIIa, GP Ib-IX-V, CD9, GPVI, CLEC-2, P2 receptors, αvβ3, GPIa-IIa, α5β1, α6β1, toll-like receptors, protease activation receptors, PGD₂, PGE₂, PAF receptors, Lysophosphatidic acid receptors, Sphingosine-1-phosphate receptors, chemokine receptors, JAMs, ICAm-2, PECAm-1, G6B, CD47, ESAM, TLT-1, CD62P, CD72, CD93, CLEC-2, C-type lectine receptors, CD63, CD84, CD151, GPI anchored proteins, glycosaminoglycan-carrying receptors, CD110, leptin receptor, Tie-1 receptor, insulin receptor, PDGF receptor, Gas6 receptors, PEAR1, Eph kinases, CD36, C1q receptor, Cd46, Serotonin Reuptake receptor, LAMP-1, LAMP-2, CD40L, CD154, PSGL-1, P2X₁, tight junction receptors, TNF receptor, Semaphorin 3A receptors, CD100, PPARγ, CD147, glutamate receptors, liver x receptors, galectin receptors.

46. The modified platelet cell of claim 44, wherein the modified platelet cell binds directly or indirectly a tumor cell, a modified or unmodified platelet cell, an endothelial cell, white blood cells, extracellular matrix, coagulation proteins or any combinations thereof.

47. The modified platelet cell of claim 44, wherein the platelet is substantially free of at least one component or component functionality required for platelet activation, cell aggregation or blood coagulation.

48. The modified platelet cell of claim 47, wherein said at least one component is a membrane lipid or molecular component.

49. The modified platelet cell of claim 44, wherein the modified platelet cell further comprises an anti-coagulant or a fibrinolytic agent.

50. The modified platelet cell of claim 49, wherein the anti-coagulant or fibrinolytic agent is encapsulated in or coated on the modified platelet cell.

51. The modified platelet cell of claim 49, wherein the modified platelet cell further comprises an inhibitor of platelet activation encapsulated in or coated on the modified platelet cell.

52. The modified platelet cell of claim 44, wherein the modified platelet further comprises an anti-cancer agent encapsulated in or coated on the modified platelet cell.

53. The modified platelet cell of claim 44, wherein the modified platelet cell further comprises an imaging agent encapsulated in or coated on the modified platelet cell.

54. The modified platelet cell of claim 44, wherein the modified platelet cell expresses at least one anti-apoptotic molecule at an increased expression level or amount relative to an unmodified or normal platelet cell.

55. The modified platelet cell of claim 44, wherein the modified platelet cell expresses an exogenous gene encoding an anti-apoptotic molecule or repress an apoptotic protein.

56. The modified platelet cell of claim 44, wherein the modified platelet cell comprises an inhibitor of a pro-apoptotic molecule or inhibitor of a molecule that promotes cell death.

57. The modified platelet cell of claim 44, wherein the modified platelet cell expresses at least one pro-apoptotic molecule or a molecule that promotes cell death at decreased/reduced expression level or amount.

58. A method comprising administering to a subject a composition comprising a modified platelet cell of claim 44.

59. The method of claim 58, wherein the subject is in need of treating, preventing or inhibiting a disease or disorder when platelet activation, aggregation and/or adhesion contributes to the pathology or symptomology of the disease.

60. The method of 58, wherein the subject is in need of: (i) treating, preventing or inhibiting tumor cell metastasis or tumor cell interactions with platelets in the circulation; (ii) enhancing, increasing, or stimulating fibrinolysis; (iii) treating, preventing or suppressing excessive platelet aggregation, blood coagulation or clotting disorders; (iv) enhancing, increasing, or stimulating clot formation at a platelet binding site; or (v) imaging platelet aggregation or blood clot formation or (vi) detecting any cells that platelet decoys can directly or indirectly bind.

61. A method of preparing a modified platelet cell, the method comprising: removing or extracting or modifying at least one membrane lipid or molecular component from a platelet, thereby obtaining a modified platelet cell that is capable of binding to a cell ligand or a platelet receptor substrate but does not undergo aggregation and/or promote blood coagulation.

62. The method of claim 61, wherein said removing or modifying comprises treating the platelets with detergent, high salt, ammonium hydroxide, and/or a fixative.

63. The method of claim 61, wherein said treating the platelets is for a period of about less than about 60 minutes.
